(19) United States
(12) Patent Application Publication
(10) Pub. No.: US 2011/0046147 A1
(43) Pub. Date: Feb. 24, 2011

(54) 17BETA-HYDROXYSTEROID DEHYDROGENASE TYPE 1 INHIBITORS FOR THE TREATMENT OF HORMONE-RELATED DISEASES

(75) Inventors: Rolf Hartmann, Saarbrucken (DE); Martin Frotscher, Sulzbach (DE); Sandrine Oberwinkler, Blieskastel (DE); Emmanuel Bey, Forbach (FR)

Correspondence Address: Briscoe, Kurt G.;Norris McLaughlin & Marcus, PA, 875 Third Avenue, 8th Floor, New York, NY, 10022, US

(73) Assignee: UNIVERSITAT DES SAARLANDES, Saarbrucken (DE)

(21) Appl. No.: 12/675,065

(22) PCT Filed: Aug. 22, 2008
(86) PCT No.: PCT/EP08/61033

§ 371 Date: Apr. 1, 2010

(30) Foreign Application Priority Data
Aug. 25, 2007 (DE) 102007040243.2

(51) Int. Cl. A61K 31/4965; A61K 31/4174; C07D 233/84; C07D 233/64; C07D 263/32; A61K 31/421; C07D 231/12; A61K 31/415; C07D 261/08; A61K 31/42; C07D 249/04; A61K 31/4192; C07D 277/24; A61K 31/426; C07D 333/16; A61K 31/381; C07D 271/06; A61K 31/4245; C07D 285/08; A61K 31/4196; C07D 211/00; A61K 31/435; C07D 241/12; C07D 257/08; A61K 31/395; A61P 5/24; A61P 35/00

(52) U.S. Cl. 514/252.1; 514/392; 548/325.1; 548/343.5; 514/399; 548/235; 514/374; 548/377.1; 514/406; 548/247; 514/378; 548/256; 514/359; 548/203; 514/365; 549/78; 514/438; 548/131; 514/364; 548/128; 514/363; 546/344; 514/277; 544/336; 544/179; 514/183

(57) ABSTRACT

The invention relates to 17beta-hydroxysteroid dehydrogenase type 1 (17betaHSD1) inhibitors, the preparation thereof and the use thereof for the treatment and prophylaxis of hormone-related, especially estrogen-related or androgen-related, diseases.

The invention relates to 17beta-hydroxysteroid dehydrogenase type 1 (17betaHSD1) inhibitors, the preparation thereof and the use thereof for the treatment and prophylaxis of hormone-related, especially estrogen related or androgen-related, diseases.

### BACKGROUND OF THE INVENTION

Steroid hormones are important chemical carriers of information serving for the long-term and global control of cellular functions. They control the growth and the differentiation and function of many organs. However, in addition to such physiological functions, they also have negative effects: they may favor the pathogenesis and proliferation of diseases in the organism, such as mammary and prostate cancers (Deroo, B. J. et al., 3. Clin. Invest., 116: 561-570 (2006); Fernandez, S. V. et al., Int. 3. Cancer, 118: 1862-1868 (2006)).

Within the scope of the biosynthesis of steroids, sex hormones are produced in the testes or ovaries. In contrast, the production of glucocorticoids and mineral corticoids takes place in the adrenal glands. Moreover, individual synthetic steps also occur outside the glands, namely in the brain or in the peripheral tissue, e.g., adipose tissue (Bulun, S. E. et al., 3. Steroid Biochem. Mol. Biol., 79: 19-25 (2001); Gangloff, A. et al., Biochem. J., 356: 269-276 (2001)). In this context, Labrie coined the term “intracrinology” in 1988 (Labrie, C. et al., Endocrinology, 123: 1412-1417 (1988); Labrie, F. et al., Ann. Endocrinol. (Paris), 56: 23-29 (1995); Labrie, F. et al., Harm. Res., 54: 218-229 (2000)). Attention was thus focused on the synthesis of steroids that are formed locally in peripheral tissues and also display their action there without getting into the blood circulation. The intensity of the activity of the hormones is modulated in the target tissue by means of various enzymes.

Thus, it could be shown that the 17β-hydroxysteroid dehydrogenase type 1 (17β-HSD1), which catalyzes the conversion of estrone to estradiol, is more abundant in endometriotic tissue and breast cancer cells while there is a deficiency in 17β-HSD type 2, which catalyzes the reverse reaction (Bulun, S. E. et al., J. Steroid Biochem. Mol. Biol., 79: 19-25 (2001); Miyoshi, Y. et al., Int. J. Cancer, 94; 685-689 (2001)).

A major class of steroid hormones is formed by the estrogens, the female sex hormones, whose biosynthesis takes place mainly in the ovaries and reaches its maximum immediately before ovulation. However, estrogens also occur in the adipose tissue, muscles and some tumors. Their main functions include a genital activity, i.e., the development and maintenance of the female sexual characteristics as well as an extragenital lipid-anabolic activity leading to the development of subcutaneous adipose tissue. In addition, they are involved in the pathogenesis and proliferation of estrogen-related diseases, such as endometriosis, endometrial carcinoma, adenomyosis and breast cancer (Bulun, S. E. et al., 3. Steroid Biochem. Mol. Biol., 79: 19-25 (2001); Miyoshi, Y. et al., Int. J. Cancer, 94: 685-689 (2001); Gunnarsson, C. et al., Cancer Res., 61: 8448-8451 (2001); Kitawaki, J. Journal of Steroid Biochemistry & Molecular Biology, 83: 149-155 (2003); Vihko, P. et al., J. Steroid. Biochem. Mol. Biol., 83: 119-122 (2002); Vihko, P. et al., Mol. Cell. Endocrinol., 215: 83-88 (2004)).

The most potent estrogen is estradiol (E₂), which is formed in premenopausal females, mainly in the ovaries. On an endocrine route, it arrives at the target tissues, where it displays its action by means of an interaction with the estrogen receptor (ER) α. After the menopause, the plasma E₂ level decreases to 1/10 of the estradiol level found in premenopausal females (Santner, S. J. et al., J. Clin. Endocrinol. Metab., 59: 29-33 (1984)). E₂ is mainly produced in the peripheral tissue, e.g., breast tissue, endometrium, adipose tissue and skin, from inactive precursors, such as estrone sulfate (E₁-S), dehydroepiandrosterone (DHEA) and DHEA-S. These reactions occur with the participation of various steroidogenic enzymes (hydroxysteroid dehydrogenases, aromatase), which are in part more abundantly produced in the peripheral tissue, where these active estrogens display their action. As a consequence of such intracrine mechanism for the formation of E₂, its concentration in the peripheral tissue, especially in estrogen-related diseases, is higher than that in the healthy tissue. Above all, the growth of many breast cancer cell lines is stimulated by a locally increased estradiol concentration. Further, the occurrence and progress of diseases such as endometriosis, leiomyosis, adenomyosis, menorrhagia, metrorrhagia and dysmenorrhea is dependent on a significantly increased estradiol level in accordingly diseased tissue.

Endometriosis is an estrogen-related disease afflicting about 5 to 10% of all females of childbearing age (Kitawaki, J., Journal of Steroid Biochemistry & Molecular Biology, 83: 149-155 (2003)). From 35 to 50% of the females suffering from abdominal pain and/or sterility show signs of endometriosis (Urdl, W., J. Reproduktionsmed. Endokrinol., 3: 24-30 (2006)). This diseases is defined as a histologically detected ectopic endometrial glandular and stromal tissue. In correspondingly severe cases, this chronic disease, which tends to relapse, leads to pain of different intensities and variable character and possibly to sterility. Three macroscopic clinical pictures are distinguished: peritoneal endometriosis, retroperitoneal deep-infiltrating endometriosis including adenomyosis uteri, and cystic ovarial endometriosis. There are various explanatory theories for the pathogenesis of endometriosis, e.g., the metaplasia theory, the transplantation theory and the theory of autotraumatization of the uterus as established by Leyendecker (Leyendecker, G. et al., Hum. Reprod., 17: 2725-2736 (2002)).

According to the metaplasia theory (Meyer, R., Zentralbl. Gynäkol., 43: 745-750 (1919); Nap, A. W. et al., Best Pract. Res. Clin. Obstet. Gynaecol., 18: 233-244 (2004)), pluripotent coelomic epithelium is supposed to have the ability to differentiate and form endometriotic foci even in adults under certain conditions. This theory is supported by the observation that endometrioses, in part severe ones, can occur in females with lacking uterus and gynastresy. Even in males who were treated with high estrogen doses due to a prostate carcinoma, an endometriosis could be detected in singular cases.

According to the theory postulated by Sampson (Halme, J. et al., Obstet. Gynecol., 64: 151-154 (1984); Sampson, J., Boston Med. Surg. J., 186: 445-473 (1922); Sampson, J., Am. J. Obstet. Gynecol., 14: 422-469 (1927)), retrograde menstruation results in the discharge of normal endometrial cells or fragments of the eutopic endometrium into the abdominal cavity with potential implantation of such cells in the peritoneal space and further development to form endometriotic foci. Retrograde menstruation could be detected as a physiological event. However, not all females with retrograde menstruation become ill with endometriosis, but various factors, such as cytokines, enzymes, growth factors, play a critical role.

The enhanced autonomous non-cyclical estrogen production and activity as well as the reduced estrogen inactivation are typical peculiarities of endometriotic tissue. This enhanced local estrogen production and activity is caused by a significant overexpression of aromatase, expression of 17β-HSD1 and reduced inactivation of potent E2 due to a lack of 17β-HSD2, as compared to the normal endometrium (Bulun, S. E. et al., J. Steroid Biochem. Mol. Biol., 79: 19-25 (2001); Kitawaki, J., Journal of Steroid Biochemistry & Molecular Biology, 83: 149-155 (2003); Karaer, O. et al., Acta. Obstet. Gynecol. Scand., 83: 699-706 (2004); Zeitoun, K. et al., J. Clin. Endocrinol. Metab., 83: 4474-4480 (1998)).

The polymorphic symptoms caused by endometriosis include any pain symptoms in the minor pelvis, back pain, dyspareunia, dysuria and defecation complaints.

One of the therapeutic measures employed most frequently in endometriosis is the surgical removal of the endometriotic foci (Urdl, W., J. Reproduktionsmed. Endokrinol., 3: 24-30 (2006)). Despite new therapeutic concepts, medicamental treatment remains in need of improvement. The purely symptomatic treatment of dysmenorrhea is effected by means of non-steroidal anti-inflammatory drugs (NSAID), such as acetylsalicylic acid, Indomethacine, ibuprofen and diclofenac. Since a COX2 overexpression could be observed both in malignant tumors and in the eutopic endometrium of females with endometriosis, a therapy with the selective COX2 inhibitors, such as celecoxib, suggests itself (Fagotti, A. et al., Hum. Reprod. 19: 393-397 (2004); Hayes, E. C. et al., Obstet. Gynecol. Surv., 57: 768-780 (2002)). Although they have a better gastro-intestinal side effect profile as compared to the NSAID, the risk of cardiovascular diseases, infarction and stroke is increases, especially for patients with a predamaged cardiovascular system (Dogne, J. M. et al., Curr. Pharm. Des., 12: 971-975 (2006)). The causal medicamental therapy is based on estrogen deprivation with related variable side effects and a generally contraceptive character. The gestagens with their anti-estrogenic and antiproliferative effect on the endometrium have great therapeutic significance. The most frequently employed substances include medroxyprogesterone acetate, norethisterone, cyproterone acetate. The use of danazole is declining due to its androgenic side effect profile with potential gain of weight, hirsutism and acne. The treatment with GnRH analogues is of key importance in the treatment of endometriosis (Rice, V.; Ann. NY Acad. Sci., 955: 343-359 (2001)); however, the duration of the therapy should not exceed, a period of 6 months since a longer term application is associated with irreversible damage and an increased risk of fracture. The side effect profile of the GnRH analogues includes hot flushes, amenorrhea, loss of libido and osteoporosis, the latter mainly within the scope of a long term treatment.

Another therapeutic approach involves the steroidal and non-steroidal aromatase inhibitors. It could be shown that the use of the non-steroidal aromatase inhibitor letrozole leads to a significant reduction of the frequency and severity of dysmenorrhea and dyspareunia and to a reduction of the endometriosis marker CA125 level (Soysal, S. et al., Hum. Reprod., 19: 160-167 (2004)). The side effect profile of aromatase inhibitors ranges from hot flushes, nausea, fatigue to osteoporosis and cardiac diseases. Long term effects cannot be excluded.

All the possible therapies mentioned herein are also employed in the combatting of diseases such as leiomyosis, adenomyosis, menorrhagia, metrorrhagia and dysmenorrhea.

Every fourth cancer disease in the female population falls under the category of mammary cancers. This disease is the main cause of death in the Western female population at the age of from 35 to 54 years (Nicholls, P. J., Pharm. J., 259: 459-470 (1997)). Many of these tumors exhibit an estrogen-dependent growth and are referred to as so-called HDBC (hormone dependent breast cancer). A distinction is made between ER+ and ER− tumors. The classification criteria are important to the choice of a suitable therapy. About 50% of the breast cancer cases in premenopausal females and 75% of the breast cancer cases in post-menopausal females are ER+ (Coulson, C., Steroid biosynthesis and action, 2nd edition, 95-122 (1994); Lower, E. et al., Breast Cancer Res. Treat., 58: 205-211 (1999)), i.e., the growth of the tumor is promoted by as low as physiological concentrations of estrogens in the diseased tissue.

The therapy of choice at an early stage of breast cancer is surgical measures, if possible, breast-preserving surgery. Only in a minor number of cases, mastectomy is performed. In order to avoid relapses, the surgery is followed by radiotherapy, or else radiotherapy is performed first in order to reduce a larger tumor to an operable size. In an advanced state, or when metastases occur in the lymph nodes, skin or brain, the objective is no longer to heal the disease, but to achieve a palliative control thereof.

The therapy of the mammary carcinoma is dependent on the hormone receptor status of the tumor, on the patient's hormone status and on the status of the tumor (Paepke, S. et al., Onkologie, 26 Suppl., 7: 4-10 (2003)). Various therapeutical approaches are available, but all are based on hormone deprivation (deprivation of growth-promoting endogenous hormones) or hormone interference (supply of exogenous hormones). However, a precondition of such responsiveness is the endocrine sensitivity of the tumors, which exists with HDBC ER+ tumors. The drugs employed in endocrine therapy include GnRH analogues, anti-estrogens and aromatase inhibitors. GnRH analogues, such as gosereline, will bind to specific membrane receptors in the target organ, the pituitary gland, which results in an increased secretion of FSH and LH. These two hormones in turn lead to a reduction of GnRH receptors in a negative feedback loop in the pituitary cells. The resulting desensitization of the pituitary cells towards GnRH leads to an inhibition of FSH and LH secretion, so that the steroid hormone feedback loop is interrupted. The side effects of such therapeutic agents include hot flushes, sweats and osteoporosis.

Another therapeutic option is the use of anti-estrogens, antagonists at the estrogen receptor. Their activity is based on the ability to competitively bind to the ER and thus avoid the specific binding of the endogenous estrogen. Thus, the natural hormone is no longer able to promote tumor growth. Today, therapeutic use involves so-called SERM (selective estrogen receptor modulators), which develop estrogen agonism in tissues such as bones or liver, but have antagonistic and/or minimal agonistic effects in breast tissue or uterus (Holzgrabe, U., Pharm. Unserer Zeit, 33: 357-359 (2004); Pasqualini, J. R., Biochim. Biophys. Acta., 1654: 123-143 (2004); Sexton, M. J. et al., Prim Care Update Ob Gyns, 8: 25-30 (2001)). Thus, these compounds are not only effective in combatting breast cancer, but also increase the bone density and reduce the risk of osteoporosis in postmenopausal females. The use of the SERM tamoxifen is most widely spread. However, after about 12-18 months of treatment, there is development of resistance, an increased risk of endometrial cancers and thrombo-embolic diseases due to the partial agonistic activity at the ER (Goss, P. E. et al., Clin. Cancer Res., 10: 5717-5723 (2004); Nunez, N. P. et al., Clin. Cancer Res., 10: 5375-5380 (2004)).

The enzymatically catalyzed estrogen biosynthesis may also be influenced by selective enzyme inhibitors. The enzyme aromatase, which converts C19 steroids to C18 steroids, was one of the first targets for lowering the estradiol level. This enzyme complex, which belongs to the cytochrome P-450 enzymes, catalyzes the aromatization of the androgenic A ring to form estrogens. The methyl group at position 10 of the steroid is thereby cleaved off. The first aromatase inhibitor employed for the therapy of breast cancer was aminogluthetimide. However, aminogluthetimide affects several enzymes of the cytochrome P-450 superfamily and thus inhibits a number of other biochemical conversions. For example, among others, the compound interferes with the steroid production of the adrenal glands so heavily that a substitution of both glucocorticoids and mineral corticoids may be necessary. In the meantime, more potent and more selective aromatase inhibitors, which can be subdivided into steroidal and non-steroidal compounds, are on the market. The steroidal inhibitors include, for example, exemestane, which has a positive effect on the bone density, which is associated with its affinity for the androgen receptor (Goss, P. E. et al., Clin. Cancer Res., 10: 5717-5723 (2004)). However, this type of compounds are irreversible inhibitors that also have a substantial number of side effects, such as hot flushes, nausea, fatigue. However, there are also non-steroidal compounds that are employed therapeutically, for example, letrozole. The advantage of these compounds resides in the lesser side effects, they do not cause uterine hypertrophy, but have no positive effect on the bone density and result in an increase of LDL (low density lipoprotein), cholesterol and triglyceride levels (Goss, P. E. et al., Clin. Cancer Res., 10: 5717-5723 (2004); Nunez, N. P. et al., Clin. Cancer Res., 10: 5375-5380 (2004)). Today, aromatase inhibitors are predominantly employed as second-line therapeutic agents. In the meantime, however, the equivalence or even superiority of aromatase inhibitors to SERM, such as tamoxifene, has been proven in clinical studies (Geisler, J. et al., Crit. Rev. Oncol. Hematol., 57: 53-61 (2006); Howell, A. et al., Lancet, 365: 60-62 (2005)). Thus, the use of aromatase inhibitors also as first-line therapeutical agents is substantiated.

However, the estrogen biosynthesis in the peripheral tissue also includes other pathways for the production of E1 and the more potent E2 by avoiding the enzyme aromatase that is locally present in the target tissue, for example, breast tumors. Two pathways for the production of estrogens in breast cancer tissue are postulated (Pasqualini, J. R., Biochim. Biophys. Acta., 1654: 123-143 (2004)), the aromatase pathway (Abul-Hajj, Y. J. et al., Steroids, 33: 205-222 (1979); Lipton, A. et al., Cancer, 59: 779-782 (1987)) and the sulfatase pathway (Perel, E. et al., J. Steroid. Biochem., 29: 393-399 (1988)). The aromatase pathway includes the production of estrogens from androgens with participation of the enzyme aromatase. The sulfatase pathway is the pathway for the production of estrone/estradiol by means of the enzyme steroid sulfatase, an enzyme that catalyzes the conversion of estrone sulfate and DHEA-S to estrone and DHEA. In this way, 10 times as much estrone is formed in the target tissue as compared to the aromatase pathway (Santner, S. J. et al., J. Clin. Endocrinol. Metab., 59: 29-33 (1984)). The estrone is then reduced by means of the enzyme 17β-HSD1 to form E2, the most potent estrogen. Steroid sulfatase and 17β-HSD1 are new targets in the battle against estrogen-related diseases, especially for the development of therapeutic agents for mammary carcinomas (Pasqualini, J. R., Biochim. Biophys. Acta., 1654: 123-143 (2004)).

Numerous steroidal sulfatase inhibitors could be found, including the potent irreversible inhibitor EMATE, which exhibited an agonistic activity at the estrogen receptor, however (Ciobanu, L. C. et al., Cancer Res., 63: 6442-6446 (2003); Hanson, S. R. et al., Angew. Chem. Int. Ed. Engl., 43: 5736-5763 (2004)). Some potent non-steroidal sulfatase inhibitors could also be found, such as COUMATE and derivatives as well as numerous sulfamate derivatives of tetrahydronaphthalene, indanone and tetralone (Hanson, S. R. et al., Angew. Chem. Int. Ed. Engl., 43: 5736-5763 (2004)). However, no sulfatase inhibitor has been able to enter the therapy of estrogen-related diseases to date.

The inhibition of 17β-HSD1, a key enzyme in the biosynthesis of E2, the most potent estrogen, could suggest itself as an option in the therapy of estrogen-related diseases in both premenopausal and postmenopausal females (Kitawaki, J., Journal of Steroid Biochemistry & Molecular Biology, 83: 149-155 (2003); Allan, G. M. et al., Mol. Cell. Endocrinol., 248: 204-207 (2006); Penning, T. M., Endocr. Rev., 18: 281-305 (1997); Sawicki, M. W. et al., Proc. Natl. Acad. Sci. USA, 96: 840-845 (1999); Vihko, P. et al., Mol. Cell. Endocrinol., 171: 71-76 (2001)). An advantage of this approach is the fact that the intervention is effected in the last step of estrogen biosynthesis, i.e., the conversion of E1 to the highly potent E2 is inhibited. The intervention is effected in the biosynthetic step occurring in the peripheral tissue, so that a reduction of estradiol production takes place locally in the diseased tissue. The use of correspondingly selective inhibitors would probably be associated with little side effects since the synthesis of other steroids would remain unaffected. It would be important that such inhibitors exhibit no or only very little agonistic activity at the ER, especially at the ER α, since agonistic binding is accompanied by an activation and thus proliferation and differentiation of the target cell. In contrast, an antagonistic activity of such compounds at the ER would prevent the natural substrates from binding at the receptor and result in a further reduction of the proliferation of the target cells. The use of selective 17β-HSD1 inhibitors for the therapy of numerous estrogen-dependent diseases is discussed, for example, for breast cancer, tumors of the ovaries, prostate carcinoma, endometrial carcinoma, endometriosis, adenomyosis. Highly interesting and completely novel is the proposal to employ selective inhibitors of 17β-HSD1 for prevention when there is a genetic disposition for breast cancer (Miettinen, M. et al., J. Mammary Gland. Biol. Neoplasia, 5: 259-270 (2000)).

Hydroxysteroid dehydrogenases (HSD) can be subdivided into different classes. The 11β-HSD modulate the activity of glucocorticoids, 3β-HSD catalyzes the reaction of Δ5-3β-hydroxysteroids (DHEA or 5-androstene-3β,17β-diol) to form ≢5-3β-ketosteroids (androstenedione or testosterone). 17β-HSD convert the less active 17-ketosteroids to the corresponding highly active 17-hydroxy compounds (androstenedione to testosterone and E₁ to E₂) or conversely (Payne, A. H. et al., Endocr. Rev., 25: 947-970 (2004); Peltoketo, H. et al., J. Mol. Endocrinol., 23: 1-11 (1999); Suzuki, T. et al., Endocr. Relat. Cancer, 12: 701-720 (2005)). Thus, the HSD play a critical role in both the activation and the inactivation of steroid hormones. Depending on the cell's need for steroid hormones, they alter the potency of the sex hormones (Penning, T. M., Endocr. Rev., 18: 281-305 (1997)), for example, E₁ is converted to the highly potent E₂ by means of 17β-HSD1, while E₂ is converted to the less potent E₁ by means of 17β-HSD2; 17β-HSD2 inactivates E₂ while 17β-HSD1 activates E₁.

To date, fourteen different 17β-HSDs have been identified (Lukacik, P. et al., Mol. Cell. Endocrinol., 1: 61-71 (2006)), and twelve of these enzymes could be cloned (Suzuki, T. et al., Endocr. Relat. Cancer, 12: 701-720 (2005)). They all belong to the so-called short chain dehydrogenase/reductase (SDR) family, with the exception of 17β-HSD5, which is a ketoreductase. The amino acid identity between the different 17β-HSDs is as low as 20-30% (Luu-The, V., J. Steroid Biochem. Mol. Biol., 76: 143-151 (2001)). The 17β-HSD family includes both membrane-bound and soluble enzymes. The X-ray structure of 6 human subtypes is known (1,3,5,10,11,13) (Ghosh, D. et al., Structure, 3: 503-513 (1995); Kissinger, C. R. et al., J. Mol. Biol., 342: 943-952 (2004); Zhou, M. et al., Acta Crystallogr. D. Biol. Crystallogr., 58: 1048-1050 (2002). The 17β-HSDs are NAD(H)-dependent and NADP(H)-dependent enzymes. They play a critical role in the hormonal regulation in humans. The enzymes are distinguished by their tissue distribution, catalytic preference (oxidation or reduction), substrate specificity and subcellular localization. The same HSD subtype was found in different tissues. It is likely that all 17β-HSDs are expressed in the different estrogen-dependent tissues, but in different concentrations. In diseased tissue, the ratio between the different subtypes is altered as compared to healthy tissue, some subtypes being overexpressed while others may be absent. This may cause an increase or decrease of the concentration of the corresponding steroid. Thus, the 17β-HSDs play an extremely important role in the regulation of the activity of the sex hormones. Further, they are involved in the development of estrogen-sensitive diseases, such as breast cancer, ovarian, uterine and endometrial carcinomas, as well as androgen-related diseases, such as prostate carcinoma, benign prostate hyperplasia, acne, hirsutism etc. It has been shown that some 17β-HSDs are also involved in the development of further diseases, e.g., pseudohermaphrodism (17β-HSD3 (Geissler, W. M. et al., Nat. Genet., 7: 34-39 (1994))), bifunctional enzyme deficiency (17β-HSD4 (van Grunsven, E. G. et al., Proc. Natl. Acad. Sci. USA, 95: 2128-2133 (1998))), polycystic kidney diseases (17β-HSD8 (Maxwell, M. M. et al., J. Biol. Chem., 270: 25213-25219 (1995))) and Alzheimer's (17β-HSD10 (Kissinger, C. R. et al., J. Mol. Biol., 342: 943-952 (2004); He, X. Y. et al., J. Biol. Chem., 274: 15014-15019 (1999); He, X. Y. et al., Mol. Cell. Endocrinol., 229: 111-117 (2005); He, X. Y. et al., J. Steroid Biochem. Mol. Biol., 87: 191-198 (2003); Yan, S. D. et al., Nature, 389: 689-695 (1997))).

The best characterized member of the 17β-HSDs is the type 1 17β-HSD. The 17β-HSD1 is an enzyme from the SDR family, also referred to as human placenta estradiol dehydrogenase (Gangloff, A. et al., Biochem. J., 356: 269-276 (2001); Jornvall, H. et al., Biochemistry, 34: 6003-6013 (1995)). Its designation as assigned by the enzyme commission is E.C.1.1.1.62.

Engel et al. (Langer, L. J. et al., J. Biol. Chem., 233: 583-588 (1958)) were the first to describe this enzyme in the 1950's. In the 1990's, first crystallization attempts were made, so that a total of 16 crystallographic structures can be recurred to today in the development of inhibitors (Alho-Richmond, S. et al., Mol. Cell. Endocrinol., 248: 208-213 (2006)). Available are X-ray structures of the enzyme alone, but also of binary and ternary complexes of the enzyme with its substrate and other ligands or substrate/ligand and cofactor.

17β-HSD1 is a soluble cytosolic enzyme. NADPH serves as a cofactor. 17β-HSD1 is encoded by a 3.2 kb gene consisting of 6 exons and 5 introns that is converted to a 2.2 kb transcript (Luu-The, V., J. Steroid Biochem. Mol. Biol., 76: 143-151 (2001); Labrie, F. et al., J. Mol. Endocrinol., 25: 1-16 (2000)). It is constituted by 327 amino acids. The molecular weight of the monomer is 34.9 kDa (Penning, T. M., Endocr. Rev., 18; 281-305 (1997)).

17β-HSD1 is expressed in the placenta, liver, ovaries, endometrium, prostate gland, peripheral tissue, such as adipose tissue and breast cancer cells (Penning, T. M., Endocr. Rev., 18: 281-305 (1997)). It was isolated for the first time from human placenta (Jarabak, J. et al., J. Biol. Chem., 237: 345-357 (1962)). The main function of 17β-HSD1 is the conversion of the less active estrone to the highly potent estradiol. However, it also catalyzes to a lesser extent the reaction of dehydroepiandrosterone (DHEA) to 5-androstene-3β,17β-diol, an androgen showing estrogenic activity (Labrie, F., Mol. Cell. Endocrinol., 78: C113-118 (1991); Poirier, D., Curr. Med. Chem., 10: 453-477 (2003); Poulin, R. et al., Cancer Res., 46: 4933-4937 (1986)). In vitro, the enzyme catalyzes the reduction and oxidation between E₁ and E₂ while it catalyzes only the reduction under physiological conditions. These bisubstrate reactions proceed according to a random catalytic mechanism, i.e., either the steroid or the cofactor is first to bind to the enzyme (Betz, G., J. Biol. Chem., 246: 2063-2068 (1971)). A catalytic mechanism in which the cofactor binds to the enzyme first is also postulated (Neugebauer, A. et al., Bioorg. Med. Chem., submitted (2005)).

The enzyme consists of a substrate binding site and a channel that opens into the cofactor binding site. The substrate binding site is a hydrophobic tunnel having a high complementarity to the steroid. The 3-hydroxy and 17-hydroxy groups in the steroid form four hydrogen bonds to the amino acid residues His221, Glu282, Ser142 and Tyr155. The hydrophobic van der Waals interactions seem to form the main interactions with the steroid while the hydrogen bonds are responsible for the specificity of the steroid for the enzyme (Labrie, F. et al., Steroids, 62: 148-158 (1997)). Like with all the other enzymes of this family, what is present as a cofactor binding site is the Rossmann fold, which is a region consisting of α-helices and sheets (β-α-β-α-β)₂, a generally occurring motif Gly-Xaa-Xaa-Xaa-Gly-Xaa-Gly, and a nonsense region Tyr-Xaa-Xaa-Xaa-Lys within the active site. What is important to the activity is a catalytic tetrade consisting of Tyr155-Lys159-Ser142-Asn114, which stabilize the steroid and the ribose in the nicotinamide during the hydride transfer (Alho-Richmond, S. et al., Mol. Cell. Endocrinol., 248: 208-213 (2006); Labrie, F. et al., Steroids, 62: 148-158 (1997); Nahoum, V. et al., Faseb. J., 17: 1334-1336 (2003)).

The gene encoding 17β-HSD1 is linked with the gene for mammary and ovarian carcinomas that is very susceptible to mutations and can be inherited, the BRCA1 gene, on chromosome 17q11-q21 (Labrie, F. et al., J. Mol. Endocrinol., 25: 1-16 (2000)). As has been demonstrated, the activity of 17β-HSD1 is higher in endometrial tissue and breast cancer cells as compared to healthy tissue, which entails high intracellular estradiol levels, which in turn cause proliferation and differentiation of the diseased tissue (Bulun, S. E. et al., J. Steroid Biochem. Mol. Biol., 79: 19-25 (2001); Miyoshi, Y. et al., Int. J. Cancer, 94: 685-689 (2001); Kitawaki, J., Journal of Steroid Biochemistry & Molecular Biology, 83: 149-155 (2003); Pasqualini, J. R., Biochim. Biophys. Acta., 1654: 123-143 (2004); Vihko, P. et al., Mol. Cell. Endocrinol., 171: 71-76 (2001); Miettinen, M. et al., Breast Cancer Res. Treat., 57: 175-182 (1999); Sasano, H. et al., J. Clin. Endocrinol. Metab., 81: 4042-4046 (1996); Yoshimura, N. et al., Breast Cancer Res., 6: R46-55 (2004)). An inhibition of 17β-HSD1 could result in the estradiol level being lowered and thus lead to a regression of the estrogen-related diseases. Further, selective inhibitors of 17β-HSD1 could be used for prevention when there is a genetic disposition for breast cancer (Miettinen, M. et al., J. Mammary Gland. Biol. Neoplasia, 5: 259-270 (2000)).

Thus, this enzyme would suggest itself as a target for the development of novel selective and non-steroidal inhibitors as therapeutic agents in the battle against estrogen-related diseases. However, there has not been a proof of concept to date.

In the literature, only a few compounds have been described as inhibitors of 17β-HSD1 (Poirier, D., Curr. Med. Chem., 10: 453-477 (2003)). Most inhibitors are steroidal compounds obtained by different variations of the estrogen skeleton (Allan, G. M. et al., 3. Med. Chem., 49: 1325-1345 (2006); Deluca, D. et al., Mol. Cell. Endocrinol., 248: 218-224 (2006); WO2006/003012; US2006/652461; WO2005/047303).

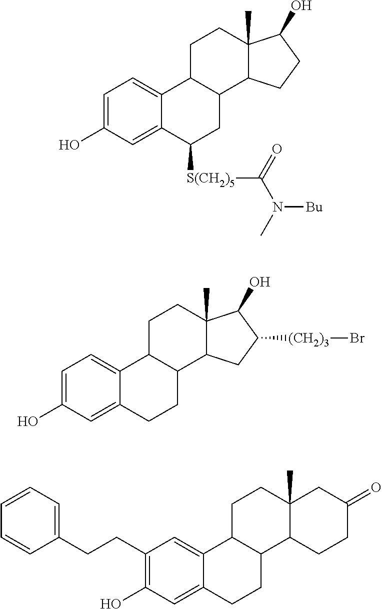

Another class of compounds which has been described is the so-called hybrid inhibitors (Qiu, W. et al., FASEB J., 16: 1829-1830 (2002); online: doi 10.1096/fj.02-0026fje), compounds that, due to their molecular structure, not only attack at the substrate binding site, but also undergo interactions with the cofactor binding site. The inhibitors have the following structure:
adenosine moiety or simplified derivatives that can interact with the cofactor binding site;
estradiol or estrone moiety, which interacts with the substrate binding site; and
a spacer of varying length as a linking element between the two moieties.

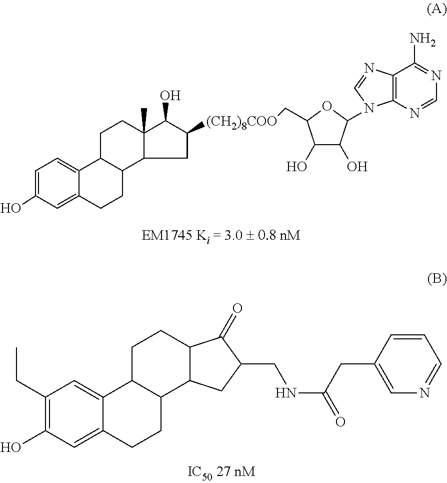

Among these compounds, inhibitors have been synthesized that exhibit a good inhibition of the enzyme and a good selectivity for 17β-HSD2 (compound B (Lawrence, H. R. et al., J. Med. Chem., 48: 2759-2762 (2005))). In addition, the inventors consider that a small estrogenic effect can be achieved by a substitution at the C2 of the steroid skeleton (Cushman, M. et al., J. Med. Chem., 38: 2041-2049 (1995); Leese, M. P. et al., J. Med. Chem., 48: 5243-5256 (2005)); however, this effect has not yet been demonstrated in tests.

However, a drawback of these steroidal compounds may be a low selectivity. With steroids, there is a risk that the compounds will also attack other enzymes of the steroid biosynthesis, which would lead to side effects. In addition, due to their steroidal structure, they may have an affinity for steroid receptors and function as agonists or antagonists.

Among the phytoestrogens, which have affinity for the estrogen receptor and act as estrogens or anti-estrogens depending on the physiological conditions, flavones, isoflavones and lignans have been tested for an inhibitory activity (Makela, S. et al., Proc. Soc. Exp. Biol. Med., 217: 310-316 (1998); Makela, S. et al., Proc. Soc. Exp. Biol. Med., 208: 51-59 (1995); Brooks, J. D. et al., J. Steroid Biochem. Mol. Biol., 94: 461-467 (2005)). Coumestrol was found to be particularly potent, but of course showed estrogenic activity (Nogowski, L., J. Nutr. Biochem., 10: 664-669 (1999)). Gossypol derivatives were also synthesized as inhibitors (US2005/0228038). In this case, however, the cofactor binding site rather than the substrate binding site is chosen as the target site (Brown, W. M. et al., Chem. Biol. Interact., 143-144, 481-491 (2003)), which might entail problems in selectivity with respect to other enzymes utilizing NAD(H) or NADP(H).

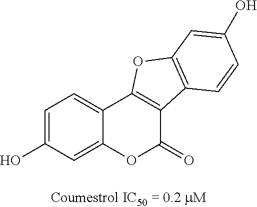

In addition to diketones, such as 2,3-butanedione and glyoxal, which were used for studies on the enzyme, suicide inhibitors were also tested. However, these were found not to be therapeutically utilizable since the oxidation rate of the alcohols to the corresponding reactive form, namely the ketones, was too weak (Poirier, D., Curr. Med. Chem., 10: 453-477 (2003)).

In other studies, Jarabak et al. (Jarabak, J. et al., Biochemistry, 8: 2203-2212 (1969)) examined various non-steroidal inhibitors for their inhibitory effect, U-11-100A having been found as the most potent compound in this group. However, as compared to other non-steroidal compounds, U-11-100A is a weak inhibitor of 17β-HSD1.

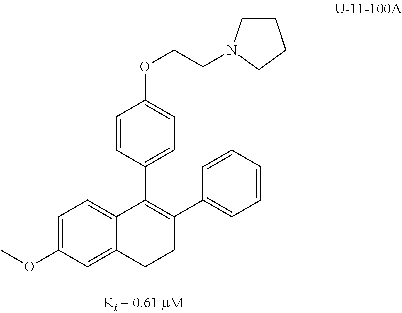

As further non-steroidal inhibitors, thiophenepyrimidinones have been examined (US2005/038053; Messinger, J. et al., Mol. Cell. Endocrinol., 248: 192-198 (2006); WO2004/110459).

Azole derivatives with two or three hydroxyphenyl substituents were presented as new estrogen receptor ligands (Fink, B. E., et al., Chem. and Biol., 6: 205-219 (1999)). The 4-alkyl-1,3,5-triarylpyrrazoles published therein are potent ligands while the bis(hydroxyphenyl)heterocycles have no affinity.

The bissubstituted azoles 2,4-bis(4-methoxyphenyl)thiazole and 4,4′-(1,3-thiazole-2,4-diyl)diphenol were already described by Fink, B. E., et al., Chem. and Biol., 6: 205-219 (1999).

WO 00/19994 describes di- and triphenyl-substituted five-membered heterocycles, wherein the phenyl radicals are unsubstituted or carry parahydroxy groups, which have a high affinity for the estrogen receptor.

Chandra, R., et al., Bioorg. & Med. Chem. Lett., 16: 1350-1352 (2006), describe 2,5-diphenyl-substituted thiophene derivatives in which the phenyl radicals have para substituents and which are suitable as β-amyloid plaque detection reagents. Demerseman, P., et al., 3. Chem. Soc., 23: 2720-2722 (1954), describe the synthesis of 2,4-bis(4-hydroxyphenyl)- and 2,4-bis(4-methoxyphenyl)-thiophene.

Muller, G. et al., Bul. Soc. Chim. France, 533-535 (1949), describe the synthesis of 2,5-diphenyl substituted pyrazines in which the phenyl radicals are substituted with acetoxy or hydroxy radicals in para and meta positions.

JP-A-03251494 employs mono- and dihydroxysubstituted terphenyl compounds as developer compounds in thermal storage materials, only one compound being mentioned that respectively has a hydroxy group at one of the outer phenyl rings, namely [1,1′:3′,1″-terphenyl]-4,4″-diol.

Guither, W. D., et al., Heterocycles, 12(6): 745-749 (1979), describe the production of 3,6-bis(3-hydroxyphenyl)-s-tetrazine.

None of the compounds stated above was described as an inhibitor of 178-HSD1.

### SUMMARY OF THE INVENTION

Estradiol is the product of the reaction catalyzed by 17β-HSD1. In addition, estradiol of all endogenous estrogens is also the steroid hormone that shows the highest affinity for the estrogen receptors (ERα and ERβ). Therefore, a great homology between the binding sites of 17β-HSD1, ERα and ERβ is to be expected. In the therapeutical approach on which the present invention is based, the inhibitors are supposed to inhibit 17β-HSD1 selectively without showing agonistic activity on the estrogen receptors.

Proceeding from the available crystal structures of 17β-HSD1, ERα and ERβ, it was believed that there are similarities in hydrophobic and hydrophilic regions. However, significant differences can also be seen. In the binding site of 17β-HSD1, there are polar amino acids (Tyr 218 and Ser 222), for which there are no analogues in the estrogen receptors. In contrast to 17β-HSD1, the estrogen receptors have no cofactor domain, so that more space is available in 17β-HSD1 at positions 15 and 16 of the steroid. The utilization of such differences is of highest importance to the design of selective 17β-HSD1 inhibitors. A wide variety of target compounds including bis(methoxyphenyl) and bis(hydroxyphenyl) substituted (hetero)aryl compounds have been synthesized, and their inhibitor activity on 17β-HSD1 and 17β-HSD2 enzymes tested in vitro in order to establish an active and selective leading structure. In addition, studies relating to the ER affinity have been performed. It has been found that certain diphenyl substituted (hetero)aryl compounds, namely those compounds in which the phenyl radicals have a meta substituent and a meta or para substituent relative to the linkage with the central (hetero)aryl, are potent inhibitors of 17β-HSD1. Thus, the invention relates to

(1) the use of compounds having the structure (I)

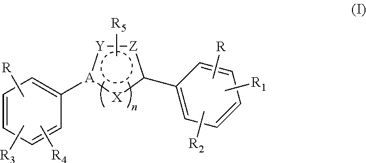

wherein
n is an integer selected from 0, 1 and 2;

### A is C or N;

X is selected from CH, S, N, NH, —HC═N—, —N═CH— and O;
Y is selected from CH, —HC═CH—, S, N, O, NH and C═S;
Z is selected from CH, N, NH and O;
R are independently selected from halogen, hydroxy, —CN, —NO₂, —N(R′)₂, —SR′, alkyl, haloalkyl, alkoxy, haloalkoxy, aryl, heteroaryl, —SO₃R′, —NHSO₂R′, —R″—NHSO₂R′, —SO₂NHR′, —R″—SO₂NHR′, —NHCOR′, —CONHR′, —R″—NHCOR′, —R″—CONHR′, —COOR′, —OOCR′, —R″—COOR′, —R″—OOCR′, —CHNR′, —SO₂R′ and —SOR′;
R₁, R₂, R₃, R₄ and R₅ independently have the meaning as stated for R or are H;
R′ is selected from H, alkyl, aryl and heteroaryl;
R″ is selected from alkylene, arylene and heteroarylene;
wherein said alkyl, alkylene, aryl, arylene, heteroaryl and heteroarylene radicals in R, R₁, R₂, R₃, R₄, R₅, R′ and R″ may be substituted with 1 to 5 radicals R′″ and wherein the radicals R′″ are independently selected from halogen, hydroxy, —CN, alkyl, alkoxy, halogenated alkyl, halogenated alkoxy, —SH, alkylsulfanyl, arylsulfanyl, aryl, heteroaryl, —COON, —COOalkyl, —CH₂OH, —NO₂ and —NH₂;
and pharmacologically acceptable salts thereof,
for the preparation of a medicament for the treatment and prophylaxis of hormone-related diseases;
(2) a compound of structure (I)

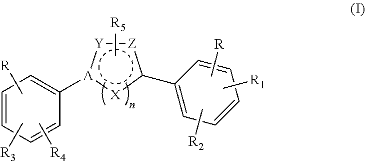

wherein
n, A, X, Y, Z, R, R_{I}, R₂, R₃, R₄ and R₅ have the meanings as stated above in (1), with the proviso that if n is 1, A is C, X is N, Y is S and Z is CH, R₁, R₂, R₃, R₄ and R₅ are H, then the radicals R are not both in para position relative to the linkage of the central (hetero)aryl group and are not at the same time OH or methoxy;
and pharmacologically acceptable salts thereof;
(3) a medicament or pharmaceutical composition containing at least one of the compounds as defined in (2) and optionally a pharmacologically suitable carrier;
(4) a process for the preparation of the compounds as defined in (2), comprising a reaction according to the following reaction scheme:

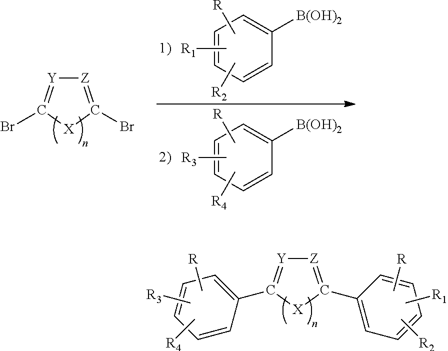

wherein the variables have the meanings as stated above in (2); and
(5) a process for the treatment and prophylaxis of hormone-related diseases in a human or animal, comprising administering a compound having a structure (I) as defined above in (1) or (2).

In particular, the two phenyl radicals having a polar group, preferably in p- or m-position relative to the central (hetero)aryl radical (such as hydroxyphenyl radicals), seem to be important to the design of the compounds of the present invention as active substances, since they mimic the hydroxy groups on positions 3 and 1.7 of estradiol and obviously serve as hydrophilic anchor sites in the 17β-HSD1 binding site. One of the phenyl radicals has to carry the polar group in m-position, while the other may carry it in m- or p-position in order to have 178-HSD1 inhibitor activity (the p-/p-substituted compounds have been shown to be no 17β-HSD1 inhibitors). The positions of the hetero atoms within the (hetero)aryl ring linking the two phenyl radicals was varied in order to examine their role in the inhibition of the enzyme. Also, the positions of the polar groups of the phenyl radicals were changed in order to find their optimum arrangement.

### DETAILED DESCRIPTION OF THE INVENTION

In the compounds of formula (I) of the invention, the variables and the terms used for their characterization have the following meanings:

“Alkyl radicals”, “haloalkyl radicals”, “alkoxy radicals” and “haloalkoxy radicals” within the meaning of the invention may be straight-chain, branched-chain or cyclic, and saturated or (partially) unsaturated. Preferable alkyl radicals and alkoxy radicals are saturated or have one or more double and/or triple bonds. Of straight-chain or branched-chain alkyl radicals, preferred are those having from 1 to 10 carbon atoms, more preferably those having from 1 to 6 carbon atoms, even more preferably those having from 1 to 3 carbon atoms. Of the cyclic alkyl radicals, more preferred are mono- or bicyclic alkyl radicals having from 3 to 15 carbon atoms, especially monocyclic alkyl radicals having from 3 to 8 carbon atoms.

“Lower alkyl radicals”, “halogenated lower alkyl radicals”, “lower alkoxy radicals” and “halogenated lower alkoxy radicals” within the meaning of the invention are straight-chain, branched-chain or cyclic saturated lower alkyl radicals and lower alkoxy radicals or those having a double or triple bond. Of the straight-chain ones, those having from 1 to 6 carbon atoms, especially 1 to 3 carbon atoms, are particularly preferred. Of the cyclic ones, those having from 3 to 8 carbon atoms are particularly preferred.

“Aryls” in the definition of R, R₁, R₂, R₃, R₄ and R₅ include mono-, bi- and tricyclic aryl radicals having from 3 to 18 ring atoms, which may optionally be anellated with one or more saturated rings. Particularly preferred are anthracenyl, dihydronaphthyl, fluorenyl, hydrindanyl, indanyl, indenyl, naphthyl, naphthenyl, phenanthrenyl, phenyl and tetralinyl.

Unless stated otherwise, “heteroaryl radicals” in the definition of R, R₁, R₂, R₃, R₄ and R₅ are mono- or bicyclic heteroaryl radicals having from 3 to 12 ring atoms and preferably having from 1 to 5 heteroatoms selected from nitrogen, oxygen and sulfur, which may be anellated with one or more saturated rings. The preferred nitrogen-containing monocyclic and bicyclic heteroaryls include benzimidazolyl, benzothiazolyl, benzoxazolyl, quinazolinyl, quinolyl, quinoxalinyl, cinnolinyl, dihydroindolyl, dihydroisoindolyl, dihydropyranyl, dithiazolyl, homopiperidinyl, imidazolidinyl, imidazolinyl, imidazolyl, indazolyl, indolyl, isoquinolyl, isoindolyl, isothiazolidinyl, isothiazolyl, isoxazolidinyl, isoxazolyl, morpholinyl, oxadiazolyl, oxazolidinyl, oxazolyl, phthalazinyl, piperazinyl, piperidyl, pteridinyl, purinyl, pyrazolidinyl, pyrazinyl, pyrazolyl, pyrazolinyl, pyridazinyl, pyridyl, pyrimidyl, pyrrolidinyl, pyrrolidin-2-onyl, pyrrolinyl, pyrrolyl, tetrazinyl, tetrazolyl, tetrahydropyrrolyl, thiadiazolyl, thiazinyl, thiazolidinyl, thiazolyl, triazinyl and triazolyl. Particularly preferred are mono- or bicyclic heteroaryl radicals with 5 to 10 ring atoms, preferably having from 1 to 3 nitrogen atoms, oxazolyl, imidazolyl, pyridyl and pyrimidyl being more preferred.

“Alkylenes”, “lower alkylenes”, “arylenes” and “heteroarylenes” within the meaning of this invention include the bivalent equivalents of the above defined alkyl, lower alkyl, aryl and heteroaryl radicals.

“Halogen” includes fluorine, chlorine, bromine and iodine.

“Pharmaceutically acceptable salts” within the meaning of the present invention include salts of the compounds with organic acids (such as lactic acid, acetic acid, amino acid, oxalic acid etc.), inorganic acids (such as HCl, HBr, phosphoric acid etc.), and, if the compounds have acid substituents, also with organic or inorganic bases. Preferred are salts with HCl.

The compounds according to embodiments (1) and (2) of the invention preferably have the following (hetero)aryl radicals as the central ring:

n is 1, A is N, X is CH, Y is C═S and Z is NH (i.e., the central ring is a 1,4-disubstituted 1,3-dihydroimidazole-2-thione);
n is 1, A is N, X is CH, Y is CH and Z is N (a 1,4-disubstituted 1H-imidazole);
n is 1, A is C, X is O or NH, Y is CH and Z is N (a 2,5-disubstituted oxazole or 1H-imidazole);
n is 1, A is C, X is N, Y is O and Z is CH (a 2,4-disubstituted oxazole);
n is 1, A is C, X is CH, Y is O and Z is N (a 3,5-disubstituted isoxazole);
n is 1, A is C, X is S, Y is N or CH and Z is CH (a 2,5-disubstituted thiazole or thiophene);
n is 1, A is C, X is N or CH, Y is S and Z is CH (a 2,4-disubstituted thiazole or thiophene);
n is 0, A is C, Y is S and Z is —HC═CH— (a 2,3-disubstituted thiophene);
n is 1, A is C, X is CH, Y and Z are N and NH (a 3,5-disubstituted 1H-pyrazole);
n is 1, A is C, X is S or O, Y and Z are N (i.e., a 2,5-disubstituted 1,3,4-thiadiazole or 1,3,4-oxadiazole);
n is 1, A is C, X and Z are N and Y is S (a 3,5-disubstituted 1,2,4-thiadiazole);
n is 2, A is C, X is CH, Y and Z are CH (a 1,4-disubstituted benzene);
n is 1, A is C, X and Y are CH and Z is —HC═CH— (a 1,3-disubstituted benzene);
n is 1, A is C, X is —N═CH—, Y is CH and Z is CH or N (a 2,5-disubstituted pyridine or pyrazine); and
n is 2, and X, Y and Z are N (i.e., a 3,6-disubstituted 1,2,4,5-tetrazine).

Of the above mentioned central rings, the thiophene, thiazole, thiadiazole, benzene, pyridine or tetrazine rings are particularly preferred.

It is preferred within the meaning of the present invention if the radicals R are independently selected from halogen, hydroxy, —CN, —NO₂, —SH, —NHR′, —SO₃R′, alkyl, haloalkyl, alkoxy, haloalkoxy, alkylsulfanyl, aryl, heteroaryl, arylsulfanyl, —NHSO₂R′, —R″—NHSO₂R′, —SO₂NHR′, —R″—SO₂NHR′, —NHCOR′, —CONHR′, —R″—NHCOR′, —R″—CONHR′, —COOR′, —OOCR′, —R″—COOR′, —R″—OOCR′, —CHNR′, —SO₂R′ and —SOR′, wherein R′ is H, lower alkyl or phenyl and R″ is lower alkylene or phenylene. Of these, preferred radicals R include halogen, hydroxy, —CN, —NO₂, —SH, —NHR′, —SO₃R′, lower alkyl, halogenated lower alkyl, lower alkoxy, halogenated lower alkoxy, (lower alkyl)sulfanyl, aryl, heteroaryl, arylsulfanyl, —NHSO₂R′, —SO₂NHR′, NHCOR′, —CONHR′, —COOR′, —OOCR′, —SO₂R′ and —SOR′ (wherein R′ is H, lower alkyl or phenyl), and it is particularly preferred if R are independently selected from halogen, hydroxy, —CN, —NO₂, —SH, —NH₂, SO₃R′, lower alkyl, halogenated lower alkyl, lower alkoxy, (lower alkyl)sulfanyl, arylsulfanyl, —NHSO₂R′, —SO₂NHR′, —NHCOR′, —CONHR′, —COOR′, —OOCR′, —SO₂R′ and —SOR′, wherein R′ is H, lower alkyl or phenyl.

Within the meaning of the invention, it is preferred that the radicals R are in meta- or para-position, namely one of R is in meta-position, and the other is in meta- or para-position relative to the linkage to the central (hetero)aryl group.

Also preferred within the meaning of the present invention if the radicals R₁, R₂, R₃, R₄ and R₅ are independently selected from H, halogen, hydroxy, —CN, lower alkyl, halogenated lower alkyl, lower alkoxy, (lower alkyl)sulfanyl, aryl, heteroaryl, arylsulfanyl, —NHSO₂R′, —SO₂NHR′, —NHCOR′, —CONHR′, —COOR′, —SO₂R′ and —SOR′, wherein R′ is H, lower alkyl or phenyl. Preferably among the mentioned radicals, they are independently selected from H, halogen, hydroxy, —CN, lower alkyl, halogenated lower alkyl, lower alkoxy, halogenated lower alkoxy, (lower alkyl)sulfanyl, —NHSO₂R′, —SO₂NHR′, —NHCOR′, —CONHR′, —COOR′, —OOCR′, —SO₂R′ and —SOR′, wherein R′ is H or lower alkyl.

Particularly preferred are those compounds in which the radicals R are independently selected from halogen, hydroxy, —CN, —COON, —NO₂, —NH₂, —SH, —SO₃H, SO₂NH₂, —NHSO₂— (lower alkyl), lower alkyl, halogenated lower alkyl, lower alkoxy and halogenated lower alkoxy, more preferably independently selected from hydroxy, —COON, —NHSO₂CH₃, —SH, —CN and C₁₋₃-alkoxy, and are in meta- or para-position, namely one in meta-position and the other in meta- or para-position (relative to the linkage to the central (hetero)aryl group). Particularly preferred are those compounds in which the radicals R₁, R₂, R₃, R₄ and R₅ are independently selected from H, halogen, halogenated lower alkyl and lower alkyl, more preferably independently selected from H, F, CF₃ and CH₃.

Compounds of structure (I) to be particularly mentioned include 4-(3-hydroxyphenyl)-1-(4-hydroxyphenyl)-1,3-dihydroimidazole-2-thione (1); 4-(4-hydroxyphenyl)-1-(3-hydroxyphenyl)-1,3-dihydroimidazole-2-thione (2); 1,4-bis-(4-hydroxyphenyl)-1,3-dihydroimidazole-2-thione (3); 3-[1-(4-hydroxyphenyl)-1H-imidazole-4-yl]phenol (4); 3-[4-(4-hydroxyphenyl)-1H-imidazole-4-yl]phenol (5); 4,4′-bis-(1H-imidazole-1,4-diyl)diphenol (6); 4,4′-(1,3-oxazole-2,5-diyl)diphenol (7); 3-[5-(4-hydroxyphenyl)-1,3-oxazole-2-yl]phenol (8); 3-[4-(4-hydroxyphenyl)-1,3-oxazole-2-yl]phenol (9); 3-[2-(4-hydroxyphenyl)-1H-imidazole-5-yl]phenol (10); 3-[5-(4-hydroxyphenyl)-1H-imidazole-2-yl]phenol (11); 4,4′-(1H-imidazole-2,5-diyl)diphenol (12); 4,4′-(1H-pyrazole-3,5-diyl)diphenol (13); 3-[3-(4-hydroxyphenyl)-1H-pyrazole-5-yl]phenol (14); 3-[5-(4-hydroxyphenyl)-1H-pyrazole-3-yl]phenol (15); 4,4′-isoxazole-3,5-diyldiphenol (16); 3-[5-(4-hydroxyphenyl)isoxazole-3-yl]phenol (17); 3-[3-(4-hydroxyphenyl)isoxazole-5-yl]phenol (18); 3-[5-(4-hydroxyphenyl)-1,3-thiazole-2-yl]phenol (19); 3-[2-(4-hydroxyphenyl)-1,3-thiazole-5-yl]phenol (20); 4,4′-(1,3-thiazole-2,5-diyl)diphenol (21); 3,3′-(1,3-thiazole-2,5-diyl)diphenol (22); 3-[4-(4-hydroxyphenyl)-1,3-thiazole-2-yl]phenol (23); 3-[2-(4-hydroxyphenyl)-1,3-thiazole-4-yl]phenol (24); 4,4′-(1,3-thiazole-2,4-diyl)diphenol (25; not covered by embodiment (2)); 3,3′-(1,3-thiazole-2,4-diyl)diphenol (26); 4,4′-thiene-2,3-diyldiphenol (27); 3-[3-(4-hydroxyphenyl)-2-thienyl]phenol (28); 3-[5-(4-hydroxyphenyl)-2-thienyl]phenol (29); 4,4′-thiene-2,5-diyldiphenol (30); 3,3′-thiene-2,5-diyldiphenol (31); 3-[5-(4-hydroxyphenyl)-3-thienyl]phenol (32); 3-[4-(4-hydroxyphenyl)-2-thienyl]phenol (33); 3,3′-thiene-2,4-diyldiphenol (34); 3,3′-(1,3,4-oxadiazole-2,5-diyl)diphenol (35); 3,3′-(1,3,4-thiadiazole-2,5-diyl)diphenol (36); 3,3′-(1,2,4-thiadiazole-2,5-diyl)diphenol (37); 3-[3-(4-methoxyphenyl)-1,2,4-thiadiazole-5-yl]phenol (38); 4,4′-(1,2,4-thiadiazole-3,5-diyl)diphenol (39); 3-[3-(4-hydroxyphenyl)-1,2,4-thiadiazole-5-yl]phenol (40); [1,1′,3′,1″]terphenyl-4,4″-diol (41); [1,1′,4′,1″]terphenyl-3,3′-diol (42); [1,1′,3′,1″]terphenyl-4,3″-diol (43); [1,1′,4′,1″]terphenyl-4,3″-diol (44); 4-[5-(3-hydroxyphenyl)-2-thienyl]-2-methylphenol (45); 4-[5-(3-hydroxyphenyl)-2-thienyl]benzene-1,2-diol (46); 2-fluoro-4-[5-(3-hydroxyphenyl)-2-thienyl]phenol (47); 2,6-difluoro-4-[5-(3-hydroxyphenyl)-2-thienyl]phenol (48); 4-[5-(3-hydroxyphenyl)-2-thienyl]-2-(trifluoromethyl)phenol (49); 3-[5-(3-fluorophenyl)-2-thienyl]phenol (50); N-{3-[5-(3-hydroxyphenyl)-2-thienyl]phenyl}methanesulfonamide (51); 3-(5-phenyl-2-thienyl)phenol (52); 3-[5-(4-hydroxyphenyl)-2-thienyl]-5-methylphenol (53); 3-[5-(4-fluorophenyl)-2-thienyl]phenol (54); 4-[5-(3-hydroxyphenyl)-3-thienyl]-2-methylphenol (55); 4-[2-(3-hydroxyphenyl)-1,3-thiazot-5-yl]-2-methylphenol (56); 3,3′-pyridine-2,5-diyldiphenol (57) and 3,3′-(1,2,4,5-tetrazine-3,6-diyl)diphenol (59), wherein compounds (19), (20), (22), (24), (26), (29), (31), (32), (33), (36), (37), (42), (45), (46), (47), (48), (49), (55), (56), (57) and (59) are particularly preferred.

In preferred embodiments of (1), (3) and (5), the above mentioned compounds of structure (I) are used for the treatment and prophylaxis of estrogen-related diseases, especially endometriosis, endometrial carcinoma, adenomyosis and breast cancer, and for the treatment of androgen-related diseases, especially prostate carcinoma and benign prostate hyperplasia (BPH).

The compounds of the present invention can be administered in any dosage form known to the skilled person, oral administration being the preferred route of administration, however.

The process for the preparation according to embodiment (4) of the invention preferably comprises a so-called Suzuki coupling. The 2,5-disubstituted thiophenes according to the present invention can be prepared according to the following synthetic route:

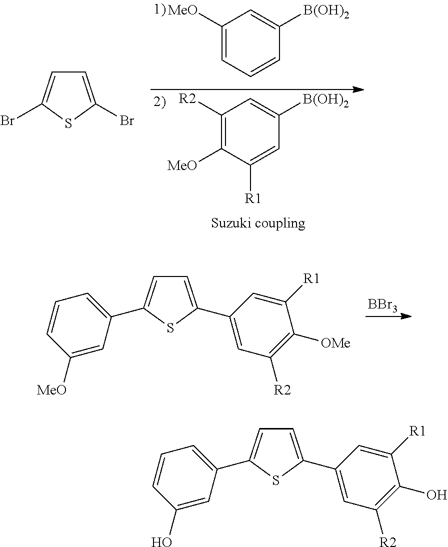

R₁═R₂=H: compound (29)
R₁=H, R₂=CH₃: compound (45)
R₁=H, R₂=OH: compound (46)
R₁=H, R₂=F: compound (47)=
R₁=H, R₂=CF₃, compound (49)
R₁=F, R₂=F: compound (48)

The quantity of active substance administered, i.e., the dose employed, depends on the kind and severity of the disease to be treated, the dosage form and therapy form, the age and constitution of the patient, and is individually adapted to the concrete situation by the attending physician within the scope of their general technical skill.

The invention is now further illustrated by means of the following Examples, which do not however limit the invention.

### EXAMPLES

### Materials and Analytical Methods

IR spectra of powders were recorded with a Bruker “Vektor 33” FT infrared spectrometer. ¹H NMR and ¹³C NMR spectra were recorded with Bruker AW-500 (500 MHz) equipment. The chemical shifts are stated in parts per million (ppm), TMS was the internal standard for recordings in CDCl₃, CD₃OD, CD₃COCD₃ and DMSO-d₆. All coupling constants (3) are stated in Hz. The mass spectra were measured with a TSQ Quantum. The reagents and solvents were obtained from commercial sources and used without any further purification. Column chromatographies were performed over silica gel (63-70 μm), the course of the reaction was monitored by means of thin-layer chromatography over Alugram SilG/UV₂₅₄ plates (Macherey-Nagel, Düren, Germany). The preparative TLC glass plates (SilG100/UV₂₅₄) were purchased from the company Macherey-Nagel. The layer thickness was 1 mm. The reactions requiring a microwave source were performed in a CEM “Discover DU5200”.

### General Synthetic Protocols:

Method A (Suzuki): One equivalent of aryl bromide, 1.2 equivalents of boric acid, 2 equivalents of a 10% sodium carbonate solution and 0.02 equivalent of palladiumtetrakis(triphenylphosphine) were dissolved Under nitrogen in 10 ml of oxygen-free toluene and heated under reflux for 18 hours. After cooling to room temperature, 20 ml of water is added. After extraction of the organic phase, the aqueous phase is washed with ethyl acetate, the combined organic phases are washed with a saturated sodium chloride solution, dried over magnesium sulfate, and the solvent is finally removed on a rotary evaporator. The raw product obtained is purified by column chromatography.

Method B (Suzuki): One equivalent of aryl bromide, 1.2 equivalents of boric acid, 2 equivalents of a 10% cesium carbonate solution and 0.02 equivalent of palladiumtetrakis(triphenylphosphine) were dissolved under nitrogen in 10 ml of oxygen-free toluene and heated under reflux for 18 hours. After cooling to room temperature, 20 ml of water is added. After extraction of the organic phase, the aqueous phase is washed with ethyl acetate, the combined organic phases are washed with a saturated sodium chloride solution, dried over magnesium sulfate, and the solvent is finally removed, on a rotary evaporator. The raw product obtained is purified by column chromatography.

Method C (Suzuki): One equivalent of aryl bromide, 1.2 equivalents of boric acid, 2 equivalents of a 10% cesium carbonate solution and 0.02 equivalent of palladiumtetrakis(triphenylphosphine) were dissolved under nitrogen in 10 ml of oxygen-free tetrahydrofuran and heated to reflux under nitrogen for 20 hours. After cooling to room temperature, 20 ml of water is added. After extraction of the organic phase, the aqueous phase is washed with ethyl acetate, the combined organic phases are washed with a saturated sodium chloride solution, dried over magnesium sulfate, and the solvent is finally removed on a rotary evaporator. The raw product obtained is purified by column chromatography.

Method D (ether cleavage): One equivalent of the dimethoxy derivative is dissolved in 10 ml of anhydrous dichloromethane. 75 equivalents of boron trifluoride/dimethyl sulfide complex is added dropwise to the reaction mixture and stirred at room temperature for 20 hours. 15 ml of Water is added to the reaction mixture, and the phases are separated. The aqueous phase is washed with 15 ml of ethyl acetate, and the combined organic phases are washed with a saturated sodium chloride solution, dried over magnesium sulfate, the solvent is removed on a rotary evaporator, and the residue is purified by preparative thin-layer chromatography.

Method E (ether cleavage): One equivalent of the dimethoxy derivative is dissolved in 10 ml of anhydrous dichloromethane and cooled down to −78° C. 6 equivalents of a 1 M boron tribromide solution is added dropwise to the reaction mixture and stirred for 20 hours. 15 ml of water is added to the reaction mixture, and the phases are separated. The aqueous phase is washed with 15 ml of ethyl acetate, and the combined organic phases are washed with a saturated sodium chloride solution, dried over sodium sulfate, the solvent is removed on a rotary evaporator, and the residue is purified by preparative thin-layer chromatography.

### Example 1

### Chemical and Physical Characterization of the Synthesized Compounds

### 1. 1-(3-Methoxyphenyl)-2-[(4-methoxyphenyl)amino]ethanone

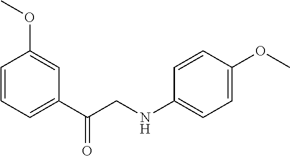

Synthesis: In cooled DMF, 1.87 mmol of p-anisidine, 1.87 mmol of 3-methoxyphenacylbromide and 1.87 mmol of triethylamine are stirred for 7 hours and then poured on ice. The precipitate obtained is filtered, dried and purified by column chromatography (hexane/ethyl acetate 6:4); yield: 70%, yellow powder, Rf: (hexane/ethyl acetate 5:5) 0.79; ¹H NMR (CDCl₃, 500 MHz): 7.55-7.57 (dt, J=1.50 Hz and J=7.80 Hz, 1H, Harom), 7.51 (m, 1H, Harom), 7.38 (t, J=7.80 Hz, 1H), 7.12-7.14 (ddd, J=0.60 Hz, J=2.50 Hz and J=8.80 Hz, 1H, Harom), 6.80 (dd, J=2.20 Hz and J=8.80 Hz, 2H, Harom), 6.66 (dd J=2.20 Hz and J=8.80 Hz, 2H, Harom), 4.54 (s, 2H), 3.85 (s, 3H, OMe), 3.73 (s, 3H, OMe); ¹³C NMR (CDCl₃, 125 MHz): 195.40, 160.00, 152.45, 141.45, 136.35, 129.85, 120.15, 120.10, 115.00, 114.35, 112.25, 55.80, 55.50, 51.45; IR: 3383, 2693, 1686, 1511, 1232, 784 cm⁻¹.

### 2. 1-(4-Methoxyphenyl)-2-(3-methoxyphenylamino)ethanone

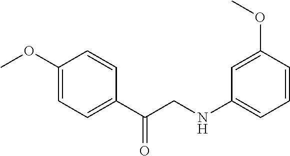

Synthesis: In cooled DMF, 1.87 mmol of m-anisidine, 4.40 mmol of 3-methoxyphenacylbromide and 4.40 mmol of triethylamine are stirred for 2 hours and then poured on ice. The precipitate obtained is filtered, dried and purified by column chromatography (hexane/ethyl acetate 6:4); yield: 70%, yellow powder. Rf: (hexane/ethyl acetate 5:5): 0.76; ¹H NMR (CDCl₃, 500 MHz): 7.97 (m, 2H, Harom), 7.12 (t, J=8.20 Hz, 1H, Harom), 6.96 (m, 2H, Harom), 6.30 (m, 2H, Harom), 6.29 (t, J=2.20 Hz, 1H, Harom), 4.54 (s, 2H), 3.87 (s, 3H, OMe), 3.78 (s, 3H, OMe); ¹³C NMR (CDCl₃, 125 MHz): 192.15, 163.10, 159.90, 147.15, 129.15, 129.05, 113.05 (2C), 105.50, 102.30, 98.50, 54.55, 49.15; IR: 3403, 1681, 1210, 827 cm⁻¹.

### 3. 1-(4-Methoxyphenyl)-2-(4-methoxyphenylamino)ethanone

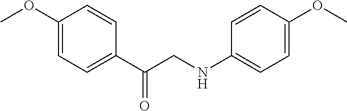

Synthesis: In cooled DMF, 8.10 mmol of p-anisidine, 8.10 mmol of 4-methoxyphenacylbromide and 8.10 mmol of triethylamine are stirred for 2 hours and then poured on ice. The precipitate obtained is filtered, dried and purified by column chromatography (hexane/ethyl acetate 6:4); yield: 98%, yellow powder. Rf (hexane/ethyl acetate 5:5): 0.78; ¹H NMR (CDCl₃, 500 MHz): 7.98 (d, J=9.10 Hz, 2H, Harom), 6.95 (d, J=9.10 Hz, 2H, Harom), 6.80 (m, 2H, Harom), 6.73 (m, 2H, Harom), 4.53 (s, 2H), 3.87 (s, 3H, OMe), 3.74 (s, 3H, OMe). ¹³C NMR (CDCl₃, 125 MHz): 193.65, 164.05, 152.80, 140.95, 130.10, 127.95, 115.05, 114.95, 114.05, 55.80, 51.35; IR: 3065, 1512, 1251, 750 cm⁻¹.

### 4. 4-(3-Methoxyphenyl)-1-(4-methoxyphenyl)-1,3-dihydroimidazole-2-thione

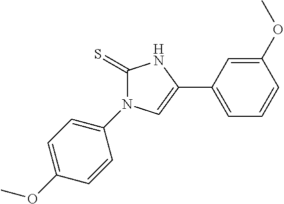

Synthesis: 6.11 mmol of 1-(3-methoxyphenyl)-2-(4-methoxyphenylamino)ethanone is dissolved in 20 ml of methanol and heated to boiling for 5 min. 6.11 mmol of potassium thiocyanate and 60 μl of concentrated hydrochloric acid are added, and the mixture is heated to boiling for 18 h. After cooling to room temperature, 50 ml of water is added. The precipitate obtained is subjected to suction filtration, dried and purified by column chromatography (hexane/ethyl acetate. 9:1); yield: 28%, white-yellow powder. Rf (ethyl acetate): 0.71; ¹H NMR (CDCl₃, 500 MHz): 7.36 (d, J=9.40 Hz, 2H, Harom) 7.26-7.30 (m, 2H, Harom), 7.10 (d, J=7.80 Hz, 2H, Harom), 6.84 (m, 1H, Harom), 6.81 (d, J=8.80 Hz, 2H, Harom), 3.82 (s, 3H, OMe), 3.72 (s, 3H, OMe); ¹³C NMR (CDCl₃, 125 MHz): 175.45, 160.05, 159.65, 129.70, 127.55, 117.45, 114.15 (2C), 113.95, 110.00, 55.45, 55.40, IR: 1626, 1514, 1222, 1037, 824 cm⁻¹; MS (APCI): 313:(M+H)⁺.

### 5. 4-(4-Methoxyphenyl)-1-(3-methoxyphenyl)-1,3-dihydroimidazole-2-thione

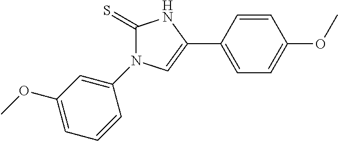

Synthesis: 2.90 mmol of 1-(4-methoxyphenyl)-2-(3-methoxyphenylamino)ethanone is dissolved in 20 ml of methanol and heated to boiling for 5 min. 2.90 mmol of potassium thiocyanate and 60 μl of concentrated hydrochloric acid are added, and the mixture is heated to boiling for 18 hours. After cooling to room temperature, 50 ml of water is added. The precipitate obtained is subjected to suction filtration, dried and purified by column chromatography (hexane/ethyl acetate 9:1). Yield: 16%, white powder, Rf (D/M 4%): 0.60. ¹H NMR (CDCl₃, 500 MHz): 7.51 (d, J=8.50 Hz, 2H), 7.38 (t, J=7.80 Hz, 1H), 7.27 (s, 1H), 7.18 (d, J=7.80 Hz, 1H, Harom), 6.97 (dd, J=2.50 Hz and J=8.50 Hz, 1H, Harom), 6.89 (d, J=8.50 Hz, 2H, Harom), 3.84 (s, 3H, OMe), 3.79 (s, 3H, OMe). ¹³C NMR (CDCl₃, 125 MHz): 188.00, 160.00, 129.95, 126.50, 118.00, 114.65 (2C), 111.80, 55.60, 55.35; IR: 3055, 1601, 1455, 1181, 825 cm⁻¹; MS (ESI): 313 (M+H)⁺.

### 6. 1,4-Bis-(4-methoxyphenyl)-1,3-dihydroimidazole-2-thione

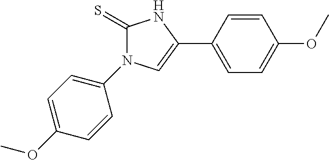

Synthesis: 7.40 mmol of 1-(4-methoxyphenyl)-2-(4-methoxyphenylamino)-ethanone is dissolved in 20 ml of methanol and heated to boiling for 5 min. 7.40 mmol of potassium thiocyanate and 60 μl of concentrated hydrochloric acid are added, and the mixture is heated to boiling for 5 h. After cooling to room temperature, 50 ml of water is added. The precipitate obtained is subjected to suction filtration, dried and purified by column chromatography (hexane/ethyl acetate 9:1). Yield: 79%, white-yellow powder; Rf (ethyl acetate): 0.77; ¹H NMR (CDCl₃+2 drops of CD₃OD, 500 MHz): 7.42 (dd, J=8.80 Hz and)=1.80 Hz, 2H, Harom), 7.39 (dd, J=8.80 Hz and J=1.80 Hz, 2H), 6.92 (m, 3H, Harom), 6.87 (dd, J=8.80 Hz and J=1.80 Hz, 2H, Harom), 3.77 (s, 3H, OMe), 3.76 (s, 3H, OMe); ¹³C NMR (CDCl₃+2 drops of CD₃OD, 125 MHz): 157.50, 157.15, 156.75, 124.95 (2C), 123.65 (2C), 112.20 (2C), 111.95 (2C), 53.15, 52.95; IR: 3373, 2958, 1673, 1512, 1237, 816 cm⁻¹. MS (APCI): 313:(M+H).⁺.

### 7. 4-(3-Hydroxyphenyl)-1-(4-hydroxyphenyl)-1,3-dihydroimidazole-2-thione (1)

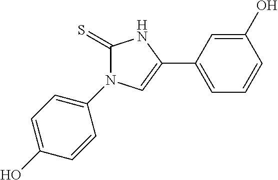

Synthesis: Prepared from 0.32 mmol of 4-(3-methoxyphenyl)-1-(4-methoxyphenyl)-1,3-dihydroimidazole-2-thione according to method D. Purification: preparative thin-layer chromatography (ethyl acetate). Yield: 61%, orange powder; Rf (ethyl acetate): 0.61; ¹H NMR (CD₃SOCD₃, 500 MHz): 12.76 (s, 1H, SH), 7.64 (s, 1H, Harom), 7.39 (d, J=8.50 Hz, 2H, Harom), 7.15-7.19 (m, 2H, Harom), 7.09 (s, 1H, Harom), 6.84 (d, J=8.50 Hz, 2H, Harom), 6.69-6.71 (m.1H, Harom). ¹³C NMR (CD₃SOCD₃, 125 MHz): 162.30, 157.60, 156.85, 129.90, 129.20, 128.95, 127.15, 116.15, 115.10 (2C), 114.85, 111.10; IR: 3214, 1604, 1514, 1395, 1101, 833, 750 cm⁻¹; MS (APCI): 283:M.⁺.

### 8. 4-(4-Hydroxyphenyl)-1-(3-hydroxyphenyl)-1,3-dihydroimidazole-2-thione (2)

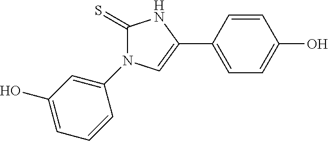

Synthesis: Prepared from 0.32 mmol of 4-(3-methoxyphenyl)-1-(4-methoxyphenyl)-1,3-dihydroimidazole-2-thione according to method D. Purification: preparative thin-layer chromatography (ethyl acetate). Yield: 37%, yellow powder; Rf (E pure): 0.59; ¹H NMR (CD₃SOCD₃, 500 MHz): 12.75 (s, 1H, SH), 7.62 (s, 1H, Harom), 7.40 (d, J=8.50 Hz, 2H, Harom), 7.13-7.18 (m, 2H, Harom), 7.07 (s, 1H, Harom), 6.83 (d, J=8.50 Hz, 2H, Harom), 6.66-6.79 (m.1H, Harom). ¹³C NMR (CD₃SOCD₃, 125 MHz): 162.35, 157.65, 156.95, 129.85, 129.00, 128.90, 127.20, 116.20 (2C), 115.05, 114.90, 111.25; IR: 3213, 1600, 1514, 1392, 1100, 845, 750 cm⁻¹; MS (APCI): 283:M.⁺.

### 9. 1,4-Bis(4-hydroxyphenyl)-1,3-dihydroimidazole-2-thione (3)

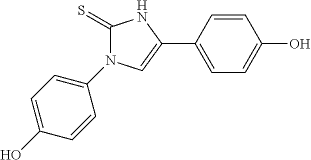

Synthesis: Prepared from 0.32 mmol of 4-(3-methoxyphenyl)-1-(4-methoxyphenyl)-1,3-dihydroimidazole-2-thione according to method D. Purification: preparative thin-layer chromatography (ethyl acetate). Yield: 36%. Rf (ethyl acetate): 0.60; ¹H NMR (CD₃OD, 500 MHz): 7.49 (d, J=8.80 Hz, 2H, Harom) 7.42 (d, J=8.80 Hz, 2H, Harom), 7.34 (s, 1H, Harom), 6.92 (d, J=8.80 Hz, 2H, Harom), 6.87 (d, J=8.80 Hz, 2H, Harom);

¹³C NMR (CD₃OD, 125 MHz): 162.00, 159.05, 158.80, 131.20, 131.10, 131.00, 128.55, 127.30, 120.55, 116.90, 116.50, 115.95; IR: 3135, 2469, 2072, 1511, 1116, 973, 836 cm⁻¹; MS (APCI): 285: (M).⁺, 286: (M+H)⁺.

### 10. 4-(3-Methoxyphenyl)-1-(4-methoxyphenyl)-1H-Imidazole

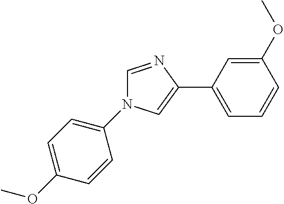

Synthesis: 0.48 mmol of 4-(3-methoxyphenyl)-1-(4-methoxyphenyl)-1,3-dihydroimidazole-2-thione is dissolved in 5 ml of cooled glacial acetic acid. 0.16 mmol of sodium nitrite is dissolved in a 33% aqueous nitric acid solution and slowly added dropwise to the reaction mixture over 20 minutes. The reaction is quenched with ammonium hydroxide. The precipitate obtained is filtered off, dried and purified by column chromatography (ethyl acetate/methanol 2%); yield: 52%, white powder; Rf: (ethyl acetate): 0.44; ¹H NMR (CDCl₃, 500 MHz): 8.90 (s, 1H, Harom) 7.62 (s, 1H, Harom), 7.56 (s, 1H, Harom), 7.46 (m, 3H, Harom), 7.35 (t, J=7.80 Hz, 1H, Harom), 7.09 (d, J=8.50 Hz, 2H, Harom), 6.97 (dd, J=1.80 Hz and J=8.20 Hz, 1H, Harom), 3.94 (s, 3H, OMe), 3.88 (s, 3H, OMe); ¹³C NMR (CDCl₃, 125 MHz): 161.10, 160.45, 136.10, 133.00, 130.50, 127.00, 123.80, 118.00, 117.10, 115.75, 111.00, 56.05, 55.80; IR: 2976, 1514, 1260, 850 cm⁻¹; MS (ESI): 281 (M+H)⁺.

### 11. 4-(4-Methoxyphenyl)-1-(3-methoxyphenyl)-1H-imidazole

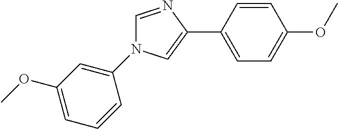

Synthesis: 0.48 mmol of 4-(4-methoxyphenyl)-1-(3-methoxyphenyl)-1,3-dihydroimidazole-2-thione is dissolved in 5 ml of cooled glacial acetic acid. 0.16 mmol of sodium nitrite is dissolved in a 33% aqueous nitric acid solution and slowly added dropwise to the reaction mixture over 20 minutes. The reaction is quenched with ammonium hydroxide, the precipitate obtained is filtered off, dried and purified by column chromatography (ethyl acetate/methanol 2%); yield: 48%, slightly yellow powder; Rf: (ethyl acetate): 0.44; ¹H NMR (CDCl₃, 500 MHz): 8.90 (s, 1H, Harom) 7.60 (s, 1H, Harom), 7.53 (s, 1H, Harom), 7.48 (m, 3H, Harom), 7.32 (t, J=7.80 Hz, 1H, Harem), 7.02 (d, J=8.50 Hz, 2H, Harom), 6.99 (dd, J=1.80 Hz and J=8.20 Hz, 1H, Harom), 3.95 (s, 3H, OMe), 3.85 (s, 3H, OMe); ¹³C NMR (CDCl₃, 125 MHz): 161.15, 160.55, 136.15, 133.10, 130.20, 127.20, 123.85, 117.90, 117.15, 115.85, 110.50, 56.25, 55.60; IR: 3200, 2966, 1520, 1255, 855 cm⁻¹; MS (ESI): 281 (M+H)⁺.

### 12. 1,4-Bis(4-methoxyphenyl)-1H-imidazole

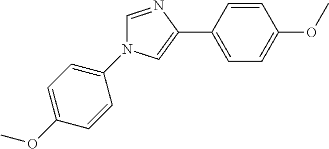

Synthesis: 0.67 mmol of 4-(4-methoxyphenyl)-1-(3-methoxyphenyl)-1,3-dihydroimidazole-2-thione is dissolved in 5 ml of cooled glacial acetic acid. 0.22 mmol of sodium nitrite is dissolved in a 33% aqueous nitric acid solution and slowly added dropwise to the reaction mixture over 20 minutes. The reaction is quenched with ammonium hydroxide. The precipitate obtained is filtered off, dried and purified by column chromatography (ethyl acetate); yield: 43%, yellow powder; Rf (ethyl acetate): 0.60; ¹H NMR (CDCl₃, 500 MHz): 8.05 (s, 1H, Harom) 7.70 (d, J=7.80 Hz, 2H, Harom), 7.40 (s, 1H, Harom), 7.33 (d, J=8.80 Hz, 2H, Harom), 6.97 (d, J=8.80 Hz, 2H, Harom), 6.90 (d, J=7.80 Hz, 2H, Harem), 3.83 (s, 3H, OMe), 3.79 (s, 3H, OMe); ¹³C NMR (CDCl₃, 125 MHz): 160.10, 115.35, 114.65, 114.55, 55.75, 55.35; IR: 2961, 2840, 1515, 1247, 1027, 828 cm⁻¹.

### 13. 3-[1-(4-Hydroxyphenyl)-1H-imidazole-4-yl]phenol (4)

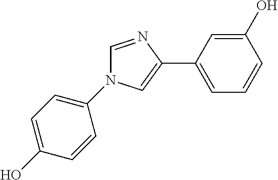

Synthesis: Prepared from 4-(3-methoxyphenyl)-1-(4-methoxyphenyl)-1H-imidazole according to method D. Purification: preparative thin-layer chromatography (ethyl acetate). Yield: 28%, yellow oil; Rf (ethyl acetate): 0.55; ¹H NMR (CD₃COCD₃, 500 MHz): 9.32 (d, J=1.20 Hz, 1H, Harom), 8.33 (d, J=1.20 Hz, 1H, Harom), 7.70 (dd, J=8.80 Hz and J=2.20 Hz, 2H, Harom), 7.33-7.36 (m, 2H, Harom), 7.29 (t, J=1.90 Hz, 1H, Harom), 7.06 (dd, J=8.80 Hz and J=2.20 Hz, 2H, Harom), 6.99 (m, 1H, Harom). ¹³C NMR (CD₃COCD₃, 125 MHz): 159.70, 158.95, 134.95, 131.60, 129.10, 128.10, 124.95, 118.25, 117.95, 117.80, 117.35, 113.35; IR: 3563, 1684, 1629, 1048, 836 cm⁻¹; MS (ESI): 253: (M).⁺.

### 14. 3-[4-(4-Hydroxyphenyl)-1H-imidazole-4-yl]phenol (5)

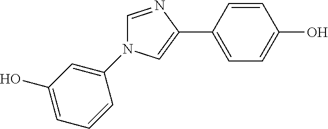

Synthesis: Prepared from 4-(4-methoxyphenyl)-1-(3-methoxyphenyl)-1H-imidazol according to method D. Purification: preparative thin-layer chromatography (ethyl acetate). Yield: 26%, yellow oil; Rf (ethyl acetate): 0.52; ¹H NMR (CD₃COCD₃, 500 MHz): 9.50 (d, J=1.50 Hz, 1H, Harom), 8.40 (d, J=1.50 Hz, 1H, Harom), 7.77 (m, 2H, Harom), 7.50 (t, J=8.20 Hz, 1H, Harom), 7.34-7.36 (m, 2H, Harom), 7.16 (dd, J=2.20 Hz and J=8.80 Hz, 1H, Harom), 7.04 (dt, J=2.20 Hz and J=8.20 Hz, 2H, Harom); ¹³C NMR (CD₃COCD₃, 125 MHz): 137.45, 132.50, 128.80 (2C), 118.35, 117.50, 114.35, 110.70; IR: 3542, 3160, 2955, 1699, 1630, 1062, 841 cm⁻¹; MS (ESI): 253: (M).⁺.

### 15. 4.4′-Bis-(1H-imidazole-1,4-diyl)diphenol (6)

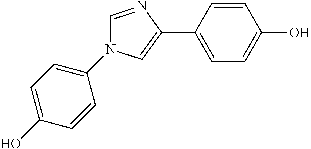

Synthesis: Prepared from 1,4-bis(4-methoxyphenyl)-1H-imidazole according to method D. Purification: preparative thin-layer chromatography (ethyl acetate). Yield: 26%, yellow powder; Rf (ethyl acetate): 0.57; ¹H NMR (CD₃COCD₃, 500 MHz): 9.43 (d, J=1.5 Hz, 1H, Harom), 8.32 (d, J=1.50 Hz, 1H, Harom), 7.74 (dd, J=8.80 Hz and J=2.20 Hz, 2H, Harom), 7.71 (dd, J=8.80 Hz and J=2.20 Hz, 2H, Harom), 7.10 (dd, J=8.80 Hz and J=2.20 Hz, 2H, Harom), 7.08 (dd, J=8.80 Hz and J=−2.20 Hz, 2H, Harom). ¹³C NMR (CD₃COCD₃, 125 MHz): 160.40, 128.70 (2C), 125.30, 117.70 (2C), 117.45 (2C), 117.25; IR: 3563, 3155, 1684, 1048, 931, 836 cm⁻¹; MS (ESI): 253: (M).⁺.

### 6. 2-Azido-1-(3-methoxyphenyl)ethanone

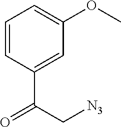

Synthesis: 3.50 mmol of 3-methoxyphenacylbromide is dissolved in 3 ml of DMF. 17.12 mmol of sodium azide is added to the reaction mixture and stirred at room temperature for 18 h. The solution is then poured on ice, stirred for one hour, filtered, the residue is additionally washed with 50 ml of water and dried over night in a desiccator. Yield: 90%, red solid; Rf (ethyl acetate): 0.55; ¹H NMR (CDCl₃, 500 MHz): 7.42-7.44 (m, 2H, Harom), 7.38 (t, J=7.80 Hz, 1H, Harom), 7.15 (ddd, J=8.20 Hz J=2.50 Hz and J=1.00 Hz, 1H, Harom), 4.53 (s, 2H, CO—CH₂), 3.85 (s, 3H, —OMe); ¹³C NMR (CDCl₃, 125 MHz): 193.05, 160.10, 135.70, 129.95, 120.60, 120.30, 112.25, 55.50, 54.95; IR: 2966, 2838, 2105, 1697, 1257, 779, 685 cm⁻¹.

### 17. 2-(3-Methoxyphenyl)-2-oxoethananium chloride

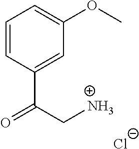

Synthesis: 8.90 mmol of 2-azido-1-(3-methoxyphenyl)ethanone is dissolved in 5 ml of absolute ethanol. 3.12 mmol of Lindiar catalyst is added and stirred under a hydrogen atmosphere for 6 hours. The mixture is filtered and 8.90 mmol of 1 M hydrochloric acid in ether solution is added dropwise to the filtrate. The hydrochloride formed is filtered off. Yield: 13%, white powder; Rf (CTZZ): 0.32; ¹H NMR (CD₃SOCD₃, 500 MHz): 8.5 (s, 3H, NH₃⁺, Cl⁻), 7.61 (dd, J=0.90 Hz and J=7.80 Hz, 1H, Harom), 7.50-7.53 (m, 2H, Harom), 7.31 (m, 1H, Harom), 4.58 (d, J=4.40 Hz, 2H, CO—CH₂), 3.85 (s, 3H, OMe). ¹³C NMR (CD₃SOCD₃, 125 MHz): 192.75, 159.50, 135.00, 130.20, 120.55, 120.45, 112.65, 55.50, 44.85. IR: 2876, 2630, 1695, 1585, 1454, 1272, 984, 784 cm⁻¹.

### 18. 3-Methoxy-N-[2-(4-methoxyphenyl)-2-oxo-ethyl]benzamide

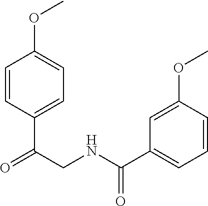

Synthesis: 4.90 mmol of 2-(4-methoxyphenyl)-2-oxoethanaminium chloride, 4.90 mmol of 3-methoxybenzoyl chloride and 9.80 mmol of triethylamine are stirred in 3 ml of dry ether at room temperature for 8 h. The reaction mixture is filtered and water is added to the filtrate. The precipitate formed is filtered off and dried over night in a desiccator. Yield: 95%, yellow powder; Rf (hexane/ethyl acetate 5:5): 0.26; ¹H NMR (CD₃COCD₃, 500 MHz): 8.10-8.05 (dt, J=1.50 Hz and J=7.80 Hz, 1H, Harom), 7.58 (t, J=7.80 Hz, 1H), 7.51 (m, 1H, Harom), 7.20-7.18 (ddd, J=0.60 Hz and J=2.50 Hz and J=8.80 Hz, 1H, Harom), 6.70 (dd, J=2.20 Hz and J=8.80 Hz, 2H, Harom), 6.53 (dd, J=2.20 Hz and J=8.80 Hz, 2H, Harom), 4.54 (s, 2H, CO—CH₂—N), 3.85 (s, 3H, —OMe), 3.83 (s, 3H, —OMe); ¹³C NMR (CD₃COCD₃, 125 MHz): 196.20, 193.40, 162.00, 152.45, 141.45, 136.35, 129.85, 120.15, 118.10, 117.00, 114.35, 111.25, 55.80, 55.50, 45.50; IR: 3427, 2985, 2840, 1735, 1241, 1038, 840, 755 cm⁻¹.

### 19. 3-Methoxy-N[2-(4-methoxyphenyl)-2-oxoethyl]benzamide

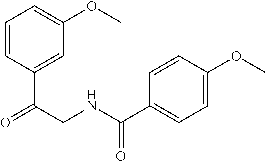

Synthesis: 4.90 mmol of 2-(3-methoxyphenyl)-2-oxoethanaminium chloride, 4.90 mmol of 4-methoxybenzoyl chloride and 9.80 mmol of triethylamine are stirred in 3 ml of dry eher at room temperature for 8 hours. The reaction mixture is filtered and water is added to the filtrate. The precipitate formed is filtered off and dried over night in a desiccator. Yield: 91%, yellow solid; Rf (hexane/ethyl acetate 5:5): 0.24; ¹H NMR (CD₃COCD₃, 500 MHz): 8.08-8.04 (dt, J=1.50 Hz and J=7.80 Hz, 1H, Harom), 7.51 (t, J=7.80 Hz, 1H), 7.47 (m, 1H, Harom), 7.20-7.18 (ddd, J=0.60 Hz and J=2.50 Hz and J=8.80 Hz, 1H, Harom), 6.75 (dd, J=2.20 Hz and J=8.80 Hz, 2H, Harom), 6.53 (dd, J=2.20 Hz and J=8.80 Hz, 2H, Harom), 4.57 (s, 2H, CO—CH₂—N), 3.83 (s, 3H, —OMe), 3.80 (s, 3H, —OMe); ¹³C NMR (CD₃COCD₃, 125 MHz): 196.10, 193.40, 161.80, 152.45, 141.50, 135.35, 129.95, 121.15, 118.10, 117.20, 114.15, 111.05, 55.90, 55.80, 46.10; IR: 3017, 2982, 2800, 1733, 1251, 1038, 840 cm⁻¹.

### 20. 4-Methoxy-N-2-(4-methoxyphenyl)-2-oxoethyl]benzamide

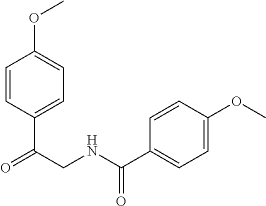

Synthesis: 4.90 mmol of 2-(4-methoxyphenyl)-2-oxoethanaminium chloride, 4.90 mmol of 4-methoxybenzoyl chloride and 9.80 mmol of triethylamine are stirred in 3 ml of dry ether at room temperature for 18 h. The reaction mixture is filtered and water is added to the filtrate. The precipitate formed is filtered off and dried over night in a desiccator. Yield 93%; white powder; Rf (hexane/ethyl acetate 5:5): 0.28; NMR (CD₃COCD₃, 500 MHz): 8.05 (d, J=7.80 Hz, 2H, Harom), 7.93 (d, J=7.80 Hz, 2H, Harom), 7.77 (s, 1H, NH), 7.06 (d, J=7.80 Hz, 2H, Harom), 7.00 (d, J=7.80 Hz, 2H, Harom), 4.84 (d, J=4.40 Hz, 2H, —CO—CH₂—N), 3.90 (s, 3H, —OMe), 3.86 (s, 3H, —OMe); ¹³C NMR (CD₃COCD₃, 125 MHz): 195.20, 192.60, 164.40, 164.45, 133.45, 132.15, 129.60 (2C), 114.15 (2C), 114.10 (2C), 54.80, 53.60, 45.40; IR: 3423, 2988, 2840, 1735, 1680, 1241, 1032, 833, 750 cm⁻¹.

### 21. 2,5-Bis(4-methoxyphenyl)oxazole

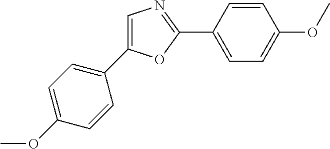

Synthesis: 0.50 mmol of 4-methoxy-N-2-(4-methoxyphenyl)-2-oxoethyl]-benzamide is admixed with 3 ml of concentrated sulfuric acid and heated to boiling for 24 h. The reaction mixture is immersed in an ice bath, and a 1 M hydrochloric acid solution is added dropwise (to pH 7). The precipitate obtained is filtered off and dried over night in a desiccator. Yield 85%, white solid; Rf (hexane/ethyl acetate 5:5): 0.55; ¹H NMR (CD₃SOCD₃, 500 MHz): 8.05 (d, J=2.50 Hz, 1H, Harom), 7.98 (d, J=8.80 Hz, 2H, Harom), 7.96 (dd, J=2.50 Hz and J=8.50 Hz, 1H, Harom), 7.58 (s, 1H, Harom), 7.11 (m, 3H, Harom), 3.83 (s, 3H, —OMe), 3.82 (s, 3H, —OMe); ¹³C NMR (CD₃SOCD₃, 125 MHz): 160.95, 159.70, 156.35, 150.20, 136.10, 127.45, 127.45, 124.20, 122.25, 119.60, 114.60 (2C), 112.55, 55.70, 55.35; IR: 2947, 2843, 1646, 1485, 1253, 1098, 828 cm⁻¹; MS (ESI): 281: (M).⁺.

### 22. 5-(4-Methoxyphenyl)-2-(3-methoxyphenyl)oxazole

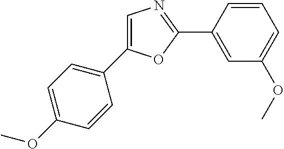

Synthesis: 1.16 mmol of 3-methoxy-N-[2-(4-methoxyphenyl)-2-oxoethyl]-benzamide, 12 ml of phosphorus oxychloride are heated to boiling in 20 ml of pyridine for 8 hours. The reaction mixture is placed into ice and diluted with 40 ml of ethyl acetate. Thereafter, it is poured into a saturated sodium hydrogencarbonate solution and extracted twice with ethyl acetate. The organic phases are dried over magnesium sulfate, filtered and purified by column chromatography (hexane/ethyl acetate 5:5); yield: 36%; white-yellowish oil; Rf: (hexane/ethyl acetate 5:5): 0.42; ¹H NMR (CD₃COCD₃, 500 MHz): 7.79 (d, J=8.80 Hz, 2H, Harom), 7.70 (dt, J=1.00 Hz and J=8.80 Hz, 1H, Harom), 7.64 (q, J=1.00 Hz, 1H, Harom), 7.53 (s, 1H, Hoxazole), 7.44 (t, J=7.90 Hz, 1H, Harom), 7.08 (m, 3H, Harom), 3.90 (s, 3H, OMe), 3.86 (s, 3H, OMe); ¹³C NMR (CD₃COCD₃, 125 MHz): 161.05, 160.95, 152.40, 130.95, 129.85, 126.65, 123.15, 121.65, 119.15, 116.95, 115.40, 111.85, 55.75, 55.70; IR: 2937.1612, 1253, 1010, 872 cm⁻¹.

### 23. 4,4′-(1,3-Oxazole-2,5-diyl)diphenol (7)

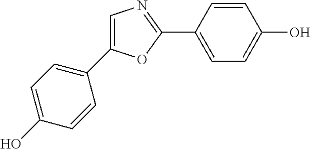

Synthesis: 0.18 mmol of 2,5-bis(4-methoxyphenyl)oxazole and 4.68 mmol of pyridinium hydrochloride are heated at 220° C. for 18 hours. After cooling to room temperature, 10 ml of water and 10 ml of ethyl acetate are added. The aqueous phase is washed twice with ethyl acetate, and the combined organic phases are dried over sodium sulfate, the solvent is filtered off and purified by preparative thin-layer chromatography (hexane/ethyl acetate: 5/5); yield: 82%, yellow solid; Rf (hexane/ethyl acetate 5/5): 0.30; ¹H NMR (CD₃OD, 500 MHz): 7.89 (d, J=7.80 Hz, 2H, Harom), 7.60 (d, J=8.80 Hz, 2H, Harom), 7.32 (s, 1H, Harom), 6.86-6.91 (m, 4H, Harom); ¹³C NMR (CD₃OD, 125 MHz): 162.35, 161.30, 159.35, 152.78, 132.80, 129.00 (2C), 126.80 (2C), 125.80 (2C), 116.90 (2C); IR: 3387, 1611, 1506, 1170, 834 cm⁻¹. MS (ESI): 254: (M+H)⁺.

### 24. 3-[5-(4-Hydroxyphenyl)-1,3-oxazole-2-yl]phenol (8)

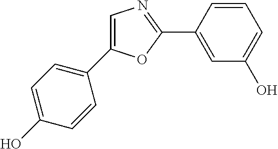

Synthesis: Prepared from 0.35 mmol of 2-(3-methoxyphenyl)-5-(4-methoxy-phenyl)oxazole according to method E. Purification: preparative thin-layer chromatography (hexane/ethyl acetate 5:5); yield: 65%, yellow solid; Rf (hexane/ethyl acetate 5/5): 0.38; ¹H NMR (CD₃COCD₃, 500 MHz): 8.80 (s, 1H, OHarom), 8.75 (s, 1H, OHarom), 7.69 (d, J=8.20 Hz, 2H, Harom), 7.60 (m, 2H, Harom), 7.46 (s, 1H, Hoxazole), 7.35 (t, J=8.20 Hz, 1H, Harom), 6.98-6.95 (m, 3H, Harom). ¹³C NMR (CD₃COCD₃, 125 MHz): 160.90, 158.95, 158.75, 152.55, 130.95, 129.80, 126.80, 122.50, 120.65, 118.25 (2C), 118.15, 116.85, 115.40, 113.55, IR: 3480, 1602, 1510, 852 cm⁻¹. MS (ESI): (M−H)⁺: 252.

### 25. 4-(3-Methoxyphenyl)-2-(4-methoxyphenyl)oxazole

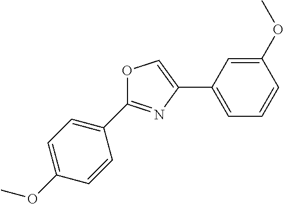

Synthesis: 3.33 mmol of 3-methoxyacetophenone, 3.99 mmol of HDNIB (hydroxy(2,4-dinitrobenzenesulfonyloxy)iodo)benzene) in acetonitrile are heated to reflux for 2 h. The reaction mixture is briefly cooled down to room temperature, and 9.99 mmol of 4-methoxybenzonitrile is added, followed by heating under reflux for 10 h. Acetonitrile is evaporated, and the solid is dissolved in dichloromethane. The organic phase is then washed with a saturated sodium hydrogencarbonate solution, dried over magnesium sulfate and purified by column chromatography (hexane/ethyl acetate 7:3). Yield 50%, white powder; Rf (hexane/ethyl acetate 6:4): 0.55; ¹H NMR (CD₃COCD₃, 500 MHz): 8.23 (s, 1H, Hoxazole), 7.90 (d, J=9.20 Hz, 2H, Harom), 7.32 (m, 2H, Harom), 7.20 (t, J=7.50 Hz, 1H, Harom), 6.93 (d, J=9.20 Hz, 2H, Harom), 6.76 (1H, Harom), 3.73 (s, 3H, OMe), 3.70 (s, 3H, OMe); IR: 3015, 2925, 1625, 789 cm⁻¹.

### 26. 3-[4-(4-Hydroxyphenyl)-1,3-oxazole-2-yl]phenol (9)

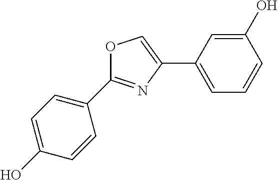

Synthesis: Prepared from 0.21 mmol of 4-(3-methoxyphenyl)-2-(4-methoxyphenyl)oxazole according to method E, purification: column chromatography (hexane/ethyl acetate 5:5); Rf (hexane/ethyl acetate 6:4): 0.62; ¹H NMR (CD₃COCD₃, 500 MHz): 8.27 (s, 1H, Hoxazole), 7.93 (d, J=8.50 Hz, 2H, Harom), 7.37 (s, 1H, Harom), 7.33 (d, J=7.60 Hz, 1H, Harom), 7.20 (t, J=7.60 Hz, 1H, Harom), 6.97 (d, J=8.50 Hz, 2H, Harom), 6.79 (m, 1H, Harom); ¹³C NMR (CD₃COCD₃, 125 MHz): 161.80, 159.70, 157.75, 141.55, 133.70, 132.90, 129.70; 128.05, 119.25, 116.70, 115.75, 114.90, 112.35; IR: 3300, 1595, 1259, 804 cm⁻¹; MS (ESI): (M−H)⁺: 282.

### 27. 2-(3-Methoxyphenyl)-5-(4-methoxyphenyl)-1H-imidazole

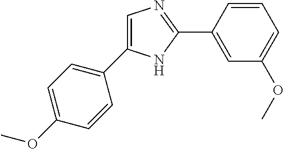

Synthesis: 0.20 mmol of 3-methoxy-N-2-(4-methoxyphenyl)-2-oxoethyl]benzamide and 1.60 mmol of ammonium acetate are dissolved in 15 ml of glacial acetic acid and heated to reflux for 2 h. The solvent is then evaporated, the solid is dissolved in ethanol and water, and 50 ml of dichloromethane is added. The organic phases are washed with a saturated sodium chloride solution, dried over magnesium sulfate and purified by column chromatography (hexane/ethyl acetate 5:5); yield: 6%, yellow solid; Rf (hexane/ethyl acetate 5:5): 0.48; NMR (CDCl₃, 500 MHz): 8.04 (s, 1H, Harom), 7.85 (d, J=8.20 Hz, 2H, Harom), 7.28-7.24 (m, 3H, Harom), 6.88 (d, J=8.20 Hz, 2H, Harom), 6.78 (dq, J=7.60 Hz and J=1.50 Hz, 1H, Harom), 3.79 (s, 3H, OMe), 3.77 (s, 3H, OMe); ¹³C NMR (CDCl₃, 125 MHz): 164.00, 132.35 (2C), 130.15, 120.60, 119.85, 113.75, 112.65, 55.60, 55.50; IR: 3077, 2965, 1678, 1468, 1240, 1031, 742 cm⁻¹; MS (ESI): 281: (M).⁻.

### 28. 2-(4-Methoxyphenyl)-5-(3-methoxyphenyl)-1H-imidazole

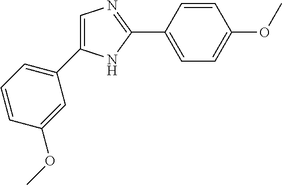

Synthesis: 0.20 mmol of 4-methoxy-N-2-(3-methoxyphenyl)-2-oxoethyl]benzamide and 1.6 mmol of ammonium acetate are dissolved in 15 ml of glacial acetic acid and heated to reflux for 2 hours. The solvent is then evaporated, the solid is dissolved in ethanol and water, and 50 ml of dichloromethane is added. The organic phases are washed with saturated sodium chloride solution, dried over magnesium sulfate and purified by column chromatography (hexane/ethyl acetate 5:5); yield: 25%, white Solid; Rf (hexane/ethyl acetate: 5/5): 0.45; ¹H NMR (CD₃SOCD₃, 500 MHz): 8.07 (d, J=2.50 Hz, 1H, Harom), 7.98 (d, J=8.50 Hz, 2H, Harom), 7.78 (d, J=8.50 Hz, 1H, Harom), 7.57 (s, 1H, Harom), 7.37 (s, 1H, Harom), 7.12-7.08 (m, 3H, Harom), 6.75 (s, 1H, Harom), 3.83 (s, 3H, OMe), 3.82 (s, 3H, OMe); ¹³C NMR (CD₃SOCD₃, 125 MHz): 162.10, 160.85, 151.40, 137.50, 128.65 (2H), 127.25, 125.40, 123.35, 120.75, 115.75 (2C), 113.70, 56.80, 56.50; IR: 3070, 2950, 1578, 1242, 742 cm⁻¹; MS (ESI): 281: (M).⁺.

### 29. 2,5-Bis(4-methoxyphenyl)-1H-imidazole

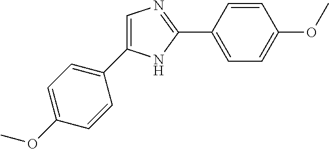

Synthesis: 0.20 mmol of 4-methoxy-N-2-(4-methoxyphenyl)-2-oxo-ethyl]benzamide and 1.60 mmol of ammonium acetate are dissolved in 15 ml of glacial acetic acid and heated to reflux for 2 hours. The solvent is then evaporated, and the solid is dissolved in ethanol and water, and 50 ml of dichloromethane was added. The organic phases are washed with saturated sodium chloride solution, dried over magnesium sulfate and purified by column chromatography (hexane/ethyl acetate 5:5); yield: 32%, yellow solid; Rf (hexane/ethyl acetate): 0.51; ¹H NMR (CD₃COCD₃, 500 MHz): 8.03 (d, J=9.10 Hz, 2H, Harom), 7.80 (d, J=8.50 Hz, 2H, Harom), 7.43 (s, 1H, Harom), 7.02 (d, J=8.80 Hz, 2H, Harom), 6.95 (d, J=9.10 Hz, Harom), 3.84 (s, 3H, OMe), 3.81 (s, 3H, OMe); ¹³C NMR (CDCl₃, 125 MHz): 161.00, 159.45, 145.05, 130.35, 129.00 (2C), 128.00 (2C), 121.25, 117.75 (2C), 114.00 (2C), 55.70, 55.20; IR: 1672, 1394, 1149, 874 cm⁻¹.

### 30. 3-[2-(4-Hydroxyphenyl)-1H-imidazole-5-yl]phenol (10)

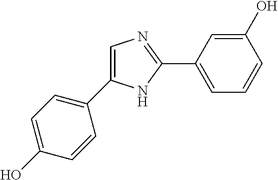

Synthesis: Prepared from 0.14 mmol of 2-(3-methoxyphenyl)-5-(4-methoxyphenyl)-1H-imidazole according to method D. Purification: preparative thin-layer chromatography (hexane/ethyl acetate: 5/5); yield: 45%, yellow solid; Rf (hexane/ethyl acetate 5:5): 0.58; ¹H NMR (CD₃COCD₃, 500 MHz): 8.58 (s, 1H, Harom), 7.42 (t, J=7.80 Hz, 2H, Harom), 7.42 (m, 1H, Harom), 7.33 (m, 1H, Harom), 7.27 (t, J=7.80 Hz, 2H, Harom), 7.05 (dd, J=0.90 Hz and J=1.50 Hz, 1H, Harom), 6.90 (dd, J=0.90 Hz and J=1.50 Hz, 1H, Harom), 6.47 (s, 1H, N—H arom); ¹³C NMR (CD₃COCD₃, 125 MHz): 168.95, 168.90, 158.25, 137.90, 136.95, 130.10 (2C), 119.40, 119.10, 118.70, 115.25; IR: 3450, 2950, 1604, 1580, 785 cm⁻¹. MS (ESI): (M+H)⁺: 253.

### 31. 3-[5-(4-Hydroxyphenyl)-1H-imidazole-2-yl]phenol (11)

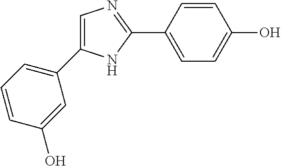

Synthesis: Prepared from 0.14 mmol of 2-(4-methoxyphenyl)-5-(3-methoxyphenyl)-1H-imidazole according to method D. Purification: preparative thin-layer chromatography (hexane/ethyl acetate: 5/5); yield: 42%, yellow solid; Rf (hexane/ethyl acetate 5:5): 0.58; ¹H NMR (CD₃COCD₃, 500 MHz): 8.56 (s, 1H, Harom), 7.40 (t, J=7.80 Hz, 2H, Harom), 7.39 (m, 1H, Harom), 7.37 (m, 1H, Harom), 7.25 (t, J=7.80 Hz, 2H, Harom), 6.99 (dd, J=0.90 Hz and J=1.50 Hz, 1H, Harom), 6.97 (dd, J=0.90 Hz and J=1.50 Hz, 1H, Harom), 6.47 (s, 1H, N-Harom); ¹³C NMR (CD₃COCD₃, 125 MHz): 168.95, 168.90, 158.25, 137.95, 136.90, 130.15 (2C), 119.30 (2C), 118.95, 115.40; IR: 3350, 3045, 2922, 1664, 1582, 760 cm⁻¹. MS (ESI): (M+H)⁺: 253.

### 32. 4,4′-(1H-Imidazole-2,5-diyl)diphenol (12)

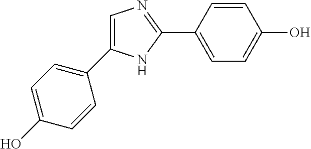

Synthesis: Prepared from 0.29 mmol of 2-(4-methoxyphenyl)-5-(3-methoxyphenyl)-1H-imidazole according to method D. Purification: preparative thin-layer chromatography (dichloromethane/methanol 1%); yield: 17%, yellow-brown solid; Rf (ethyl acetate): 0.30; ¹H NMR (CD₃OD, 500 MHz): 7.83 (d, J=8.70 Hz, 2H, Harom), 7.62 (d, J=8.70 Hz, 2H, Harom), 7.60 (s, 1H, Harom), 7.03 (d, J=8.70 Hz, 2H, Harom), 6.92 (d, J=8.70 Hz, 2H, Harom); ¹³C NMR (CD₃OD, 125 MHz): 131.30, 129.15, 120.15, 120.15, 119.80, 119.80, 115.55 (2C), 114.75 (2C), 114.30; IR: 2590, 1645, 1488, 1114, 841 cm⁻¹; MS (ESI): 253:(M+H)⁺.

### 33. 1-(3-Methoxyphenyl)-3-(4-methoxyphenyl)propenone

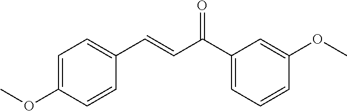

Synthesis: To a freshly prepared sodium ethanolate solution, 7.30 mmol of 3-methoxyacetophenone and 7.30 mmol of 4-methoxybenzaldehyde are added at room temperature and stirred for 2 hours. The ethanol is evaporated, and the reaction mixture is purified by column chromatography (hexane/ethyl acetate 7:3); yield: 33%, yellow oil; Rf (hexane/ethyl acetate 5:5): 0.72; ¹H NMR (CDCl₃, 500 MHz): 7.61-7.58 (d, J=15.40 Hz, 1H, Hethylen), 7.42 (d, J=8.80 Hz, 2H, Harom), 7.38-7.36 (m, 3H, Harom), 7.24-7.20 (d, J=15.40 Hz, 1H, Hethylen), 7.18 (t, J=8.10 Hz, 1H, Harom), 6.91 (dd, J=8.10 Hz and J=2.00 Hz, 1H, Harom), 6.71 (d, J=8.80 Hz, 2H, Harom), 3.64 (s, 3H, OMe), 3.58 (s, 3H, OMe); ¹³C NMR (CDCl₃, 125 MHz): 190.90, 162.75, 160.90, 145.60, 140.90, 131.30 (2C), 130.55, 128.60, 122.00, 120.65, 119.90 (2C), 114.00, 56.35, 56.30; IR: 1735, 1658, 1571, 1280, 1170, 1025, 791 cm⁻¹.

### 34. 1-(4-Methoxyphenyl)-3-(3-methoxyphenyl)propenone

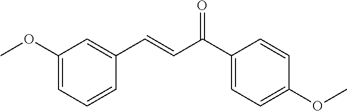

Synthesis: To a freshly prepared sodium ethanolate solution, 7.30 mmol of 3-methoxyacetophenone and 7.30 mmol of 4-methoxybenzaldehyde are added at room temperature and stirred for 2 hours. The ethanol is evaporated, and the reaction mixture is purified by column chromatography (hexane/ethyl acetate 7:3); yield: 75%; white powder; Rf (hexane/ethyl acetate 5:5): 0.89; ¹H NMR (CDCl₃, 500 MHz): 8.00 (d, J=8.80 Hz, 2H, Harom), 7.74-7.70 (d, J=15.50 Hz, 1H, Hethylen), 7.50-7.46 (d, J=15.50 Hz, 1H, Hethylen), 7.29 (t, J=8.10 Hz, 1H, Harom), 7.21 (d, J=8.10 Hz, 1H, Harom), 7.11 (m, 1H, Harom), 6.96-6.94 (m, 3H, Harom), 3.85 (s, 3H, OMe), 3.82 (s, 3H, OMe); ¹³C NMR (CDCl₃, 125 MHz): 189.05, 163.70, 160.15, 144.15, 136.65, 131.25 (2C), 130.10, 122.45, 121.20, 116.30 (2C), 114.05, 113.65, 58.40, 55.70; IR: 1657, 1592, 1251, 1166, 1018, 830 cm⁻¹.

### 35. 1,3-Bis(4-methoxyphenyl)propenone

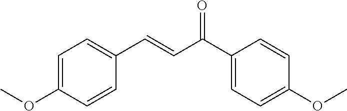

Synthesis: To a freshly prepared sodium ethanolate solution, 7.30 mmol of 3-methoxyacetophenone and 7.30 mmol of 4-methoxybenzaldehyde are added at room temperature and stirred for 2 hours. The ethanol is evaporated, and the reaction mixture is purified by column chromatography (hexane/ethyl acetate 7:3); yield: 98%, white powder; Rf (hexane/ethyl acetate 5:5): 0.80; ¹H NMR (CDCl₃, 500 MHz): 8.05 (d, J=8.80 Hz, 2H, Harom), 7.75 (d, J=15.50 Hz, 1H, Hethylen), 7.48 (d, J=15.50 Hz, 1H, Hethylen), 7.30 (d, J=8.80 Hz, 2H, Harom), 7.19 (d, J=8.20 Hz, 2H, Harom), 6.89 (d, J=8.20 Hz, 2H, Harom), 3.84 (s, 3H, OMe), 3.79 (s, 3H, OMe); ¹³C NMR (CDCl₃, 125 MHz): 189.20, 162.70, 160.10, 145.20, 131.10 (2C), 130.20, 122.60, 121.10, 117.10, 116.30, 114.05, 113.65, 58.60, 55.80; IR: 2980, 1687, 1552, 1251, 850 cm⁻¹.

### 36. 3,5-Bis(4-methoxyphenyl)-1H-pyrazole

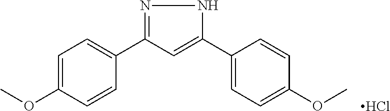

Synthesis: 0.94 mmol of 1,3-bis(4-methoxyphenyl)propane-1,3-dione is dissolved in a THF/DMF mixture (1:3). 0.94 mmol of hydrazine monohydrate is added dropwise and heated to reflux for 18 hours. After cooling to room temperature, 10 ml of a saturated lithium chloride solution and 10 ml of ethyl acetate are added. The organic phase is washed with saturated sodium chloride solution, dried over magnesium sulfate and concentrated. 0.94 mmol of a 1 M hydrochloric acid solution in ether is added. The precipitate formed is subjected to suction filtration and washed with ether. Yield: 91%, white powder; Rf (ethyl acetate): 0.48, ¹H NMR (CDCl₃, 500 MHz): 7.79 (d, J=8.20 Hz, 4H, Harom), 7.09 (s, 1H, Harom), 7.03 (d, J=8.20 Hz, 4H, Harom), 3.80 (s, 6H, OMe). ¹³C NMR (CDCl₃, 125 MHz): 159.55, 127.00, 114.45, 98.80, 55.40, IR: 3009, 2944, 2577, 1619, 1518, 1265, 803 cm⁻¹.

### 37. 3-(4-Methoxyphenyl)-5-(3-methoxyphenyl)pyrazole

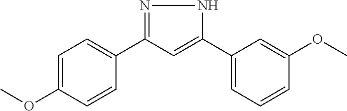

Synthesis: 0.93 mmol of 1-(4-methoxyphenyl)-3-(3-methoxyphenyl) propenone is dissolved in ethanol. 3.72 mmol of hydrazine monohydrate and 3.72 mmol of glacial acetic acid are added dropwise. The mixture is heated to reflux for 24 hours. After cooling to room temperature, the precipitate is filtered off. Water and ethyl acetate are added to the filtrate. The organic phase is washed with saturated sodium chloride solution, dried over magnesium sulfate and purified first by column chromatography (hexane/ethyl acetate 5:5) and then by preparative thin-layer chromatography (dichloromethane/methanol 1%). Yield: 25%, yellow oil; Rf (dichloromethane/methanol 1%): 0.18; ¹H NMR (CDCl₃, 500 MHz): 7.55 (d, J=8.80 Hz, 2H, Harom), 7.21-7.19 (m, 2H, Harom), 7.18 (t, J=7.80 Hz, 1H, Harom), 6.78-6.75 (m, 3H, Harom), 6.62 (s, 1H, Harom), 3.74 (s, 3H, OMe), 3.62 (s, 3H, OMe); ¹³C NMR (CDCl₃, 125 MHz): 159.85, 159.55, 129.70, 126.85, 126.85, 118.10, 114.15 (2C), 114.10, 110.50, 99.35, 55.20, 55.05; IR: 2933, 2837, 1601, 1439, 1250, 1033, 834 cm⁻¹.

### 38. 3-(3-Methoxyphenyl)-5-(4-methoxyphenyl)pyrazole

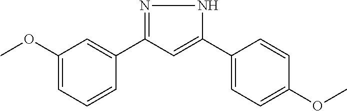

Synthesis: 1.03 mmol of 2,3-dibromo-1-(3-methoxyphenyl)-3-(4-methoxyphenyl)propenone is dissolved in ethanol. 4.12 mmol of hydrazine monohydrate and 4.12 mmol of glacial acetic acid are added dropwise. The mixture is heated under reflux for 24 hours. After cooling to room temperature, the precipitate is filtered off. Water and ethyl acetate are added to the filtrate. The organic phase is washed with a saturated sodium chloride solution, dried over magnesium sulfate and purified by column chromatography (hexane/ethyl acetate 5:5); yield: 16%, white solid; Rf (hexane/ethyl acetate 5:5): 0.70; ¹H NMR (CDCl₃, 500 MHz): 7.96 (d, J=8.80 Hz, 2H, Harom), 7.67 (t, J=2.30 Hz, 1H, Harom), 7.50 (d, J=7.80 Hz, 1H, Harom), 7.38 (t, J=7.80 Hz, 1H, Harom), 7.02-6.99 (m, 3H, Harom), 6.88 (s, 1H, Harom), 3.92 (s, 3H, OMe), 3.84 (s, 3H, OMe); ¹³C NMR (CDCl₃, 125 MHz): 161.80, 160.30, 147.60, 130.35, 128.90, 127.20, 119.50, 117.85, 114.80 (2C), 111.90, 109.80, 100.23, 55.90, 55.45; IR: 1611, 1480, 1258, 1022, 818 cm⁻¹.

### 39. 4,4′-(1H-Pyrazole-3,5-diyl)diphenol (13)

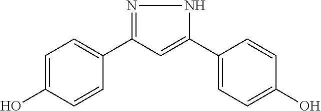

Synthesis: Prepared from 0.25 mmol of 3-(3-methoxyphenyl)-5-(4-methoxyphenyl)pyrazole according to method E. Purification: preparative thin-layer chromatography: (dichloromethane/methanol 8%). Yield: 55%, yellow powder; ¹H NMR (CD₃OD, 500 MHz): 8.59 (d, J=8.50 Hz, 4H, Harom), 7.84 (d, J=8.50 Hz, 4H, Harom), 7.76 (s, 1H, Harom); ¹³C NMR (CD₃OD, 125 MHz): 159.50, 150.50, 128.60, 124.10, 117.00, 99.80, 80.60, 69.50; IR: 3400, 3200, 1613, 1509, 1460 cm⁻¹; MS (ESI): 253.

### 40. 3-[3-(4-Hydroxyphenyl)-1H-pyrazole-5-yl]phenol (14)

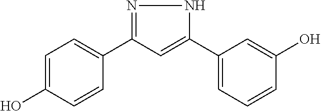

Synthesis: Prepared from 0.25 mmol of 3-(3-methoxyphenyl)-5-(4-methoxyphenyl)pyrazole according to method E. Purification: preparative thin-layer chromatography: (dichloromethane/methanol 8%). Yield: 55%, orange powder; Rf (D/M 8%): 0.25; ¹H NMR (CD₃OD, 500 MHz): 7.65 (d, J=8.50 Hz, 2H, Harom), 7.22 (m, 1H, Harom), 6.83 (d, J=8.50 Hz, 2H, Harom), 6.81 (s, 1H, Harom), 6.72-6.74 (m, 3H, Harom); ¹³C NMR (CD₃OD, 125 MHz): 160.50, 131.85, 122.10, 122.10, 117.05, 116.80, 116.00, 113.30 (2C), 102.15; IR: 3500, 2935, 1620, 790 cm⁻¹; MS (ESI): (M+H)⁺: 253.

### 41. 3-[5-(4-Hydroxyphenyl)-1H-pyrazole-3-yl]phenol (15)

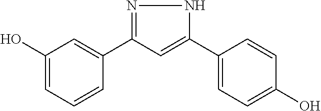

Synthesis: Prepared from 0.157 mmol of 3-(3-methoxyphenyl)-5-(4-methoxyphenyl)pyrazole according to method E. Purification: preparative thin-layer chromatography: (dichloromethane/methanol 10 Ws). Yield: 39%, orange powder; Rf (D/M 10%): 0.42; ¹H NMR (CD₃OD, 500 MHz): 7.62 (d, J=8.50 Hz, 2H, Harom), 7.24-7.21 (m, 3H, Harom), 6.85 (d, J=8.50 Hz, 2H, Harom), 6.79 (s, 1H, Harom), 6.78-6.76 (m, 1H, Harom); ¹³C NMR (CD₃OD, 125 MHz): 159.00, 130.85, 128.10, 128.10, 118.05, 116.65, 116.10, 113.55 (2C), 100.05; IR: 3411, 2925, 1614, 1459, 785 cm⁻¹; MS (ESI): (M+H)⁺: 253.

### 42. 2,3-Dibromo-1-(3-methoxyphenyl)-3-(4-methoxyphenyl)propan-1-one

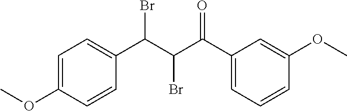

Synthesis: 1.03 mmol of 1-(3-methoxyphenyl)-3-(4-methoxyphenyl) propenone is dissolved in 5 ml of absolute ether and placed in an ice bath. 1.03 mmol of bromine is diluted in 2 ml of absolute ether and added dropwise to the mixture. After about 1 h, the yellow precipitate is filtered off and washed with ether. Yield: 97%, white solid; Rf (hexane/ethyl acetate 5:5): 0.80; ¹H NMR (CDCl₃, 500 MHz): 7.65 (d, J=8.20 Hz, 1H, Harom), 7.59 (t, J=2.30 Hz, 1H, Harom), 7.44-7.41 (m, 3H, Harom), 7.19-7.17 (dd, J=8.20 Hz and J=2.30 Hz, 1H, Harom), 6.92 (d, J=8.80 Hz, 2H, Harom), 5.76 (d, J=11.40 Hz, 1H, CH—Br), 5.63 (d, J=11.40 Hz, 1H, CH—Br), 3.88 (s, 3H, OMe), 3.82 (s, 3H, OMe); ¹³C NMR (CDCl₃, 125 MHz): 191.20, 160.20, 160.15, 135.90, 130.30, 129.90, 129.65 (2C), 121.25, 120.70, 114.25 (2C), 113.30, 55.55, 55.35, 50.25, 47.25; IR: 2966, 2840, 1684, 1597, 1254, 1022, 756 cm⁻¹.

### 43. 2,3-Dibromo-1-(4-methoxyphenyl)-3-(3-methoxyphenyl)propane-1-one

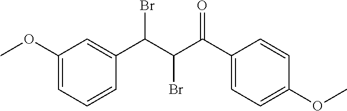

Synthesis: 0.93 mmol of 1-(3-methoxyphenyl)-3-(4-methoxyphenyl) propenone is dissolved in 5 ml of carbon tetrachloride and placed in an ice bath. 0.93 mmol of bromine is diluted in 2 ml of carbon tetrachloride and added dropwise to the mixture. After about 1.5 h, the solvent is evaporated. Yield: 96%, brown oil; Rf (dichloromethane): 0.72; ¹H NMR (CDCl₃, 500 MHz): 8.06 (d, J=9.10 Hz, 2H, Harom), 7.31 (t, J=7.90 Hz, 1H, Harom), 7.11 (d, J=7.90 Hz, 1H, Harom), 7.03 (t, J=1.80 Hz, 1H, Harom), 6.99 (d, J=9.10 Hz, 2H, Harom), 6.89 (dd, J=7.88 Hz and J=1.80 Hz, 1H, Harom), 5.75 (d, J=11.40 Hz, 1H, CH—Br), 5.60 (d, J=11.40 Hz, 1H, CH—Br), 3.89 (s, 3H, OMe), 3.84 (s, 3H, OMe); ¹³C NMR (CDCl₃, 125 MHz): 189.65, 164.45, 159.70, 140.00, 131.35 (2C), 129.85, 127.25, 120.65, 114.50, 114.30, 114.25, 55.65, 55.35, 49.90, 46.70; IR: 1675, 1597, 1255, 1028, 844 cm⁻¹.

### 44. 3,5-Bis(4-methoxyphenyl)isoxazole

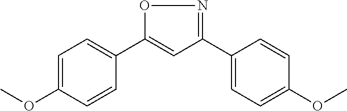

Synthesis: 4 mmol of 1,3-bis(4-methoxyphenyl)-1,3-propanedione is stirred under reflux with 4.20 mmol of hydroxylamine hydrochloride and 10 ml of absolute ethanol for 7 hours. After cooling to room temperature, the reaction mixture is poured in 50 ml of water. The precipitate formed is filtered off, washed with cool water, dried and purified by column chromatography (hexane/ethyl acetate 9:1); yield: 97%, slightly yellow powder; Rf (hexane/ethyl acetate: 8:2); ¹H NMR (CDCl₃, 500 MHz): 7.83 (s, 1H, Hisoxazol), 7.80 (d, J=8.80 Hz, 2H, Harom), 7.78 (d, J=9.10 Hz, 2H, Harom), 7.00 (d, J=9.20 Hz, 2H, Harom), 6.96 (d, J=8.80 Hz, 2H Harom), 3.87 (s, 3H, Harom), 3.85 (s, 3H, Harom); ¹³C NMR (CDCl₃, 500 MHz): 146.60, 131.80, 128.10, 129.90, 114.10, 54.00, 52.20; IR: 2920, 1603, 1501, 1254, 850 cm⁻¹.

### 45. 3-(3-Methoxyphenyl)-5-(4-methoxyphenyl)isoxazole

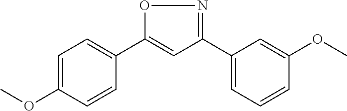

Synthesis: 0.90 mmol of 2,3-dibromo-1-(3-methoxyphenyl)-3-(4-methoxyphenyl)propane-1-one is stirred under reflux with 0.90 mmol of hydroxylamine hydrochloride and 10 ml of absolute ethanol for 24 hours. After cooling to room temperature, the reaction mixture is poured in 50 ml of water. The aqueous phase is extracted with ethyl acetate, and the resulting organic phases are washed with a saturated sodium chloride solution, dried over magnesium sulfate and purified by column chromatography (hexane/ethyl acetate 7:3); yield: 12%, slightly yellow solid; Rf (hexane/ethyl acetate 5:5): 0.72; ¹H NMR (CDCl₃, 500 MHz): 7.76 (d, J=8.20 Hz, 2H, Harom), 7.41-7.38 (m, 3H, Harom), 6.98-6.96 (m, 3H, Harom), 6.67 (s, 1H, Harom), 3.86 (s, 3H, OMe), 3.85 (s, 3H, OMe); ¹³C NMR (CDCl₃, 125 MHz): 170.40, 162.85, 161.15, 160.00, 130.55, 129.90, 127.45, 127.45, 120.30, 119.30, 116.05 (2C), 114.45, 111.75, 96.25, 55.40; IR: 3003, 2927, 2837, 1613, 1473, 1253, 1177, 836 cm⁻¹.

### 46. 2-(1H-1,2,3-Benzotriazole-1-yl)-1-(4-methoxyphenyl)ethanone

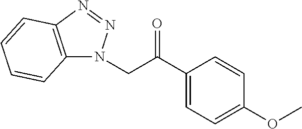

Synthesis: 4.40 mmol of benzotriazole is dissolved in 2 ml of anhydrous THF and cooled in an ice bath. 4.40 mmol of sodium hydride is added in several small portions. After 45 minutes, 4.40 mmol of 4-methoxyphenacyl bromide is added and stirred at room temperature for 18 hours. The raw product is washed with water and thereafter with a saturated sodium chloride solution, dried over magnesium sulfate and purified by column chromatography (hexane/ethyl acetate 9:1). Yield: 55%, white powder; Rf (hexane/ethyl acetate 5:5): 0.38; ¹H NMR (CDCl₃, 500 MHz): 8.09 (d, J=8.20 Hz, 1H, Harom), 8.00 (d, J=9.15 Hz, 2H, Harom), 7.45-7.38 (m, 2H, Harom), 7.36-7.34 (m, 1H, Harom), 7.95 (d, J=9.15 Hz, 2H, Harom), 6.00 (s, 2H, CH₂—CO), 3.86 (s, 3H, OMe); ¹³C NMR (CDCl₃, 125 MHz): 189.80, 165.60, 147.15, 134.90, 131.75, 131.75, 128.75 (2C), 128.10, 125.00, 121.10, 115.40, 110.70, 56.65, 54.65; IR: 2966, 2931, 1688, 1601, 1239, 1169, 824, 756 cm⁻¹.

### 47. 2-(1H-1,2,3-Benzotriazole-1-yl)-1-(3-methoxyphenyl)-3-(4-methoxyphenyl)prop-2-ene-1-one

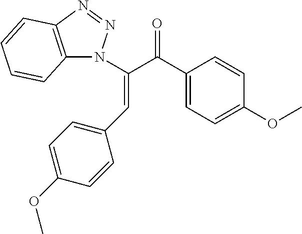

Synthesis: 0.56 mmol of 2-(1H-1,2,3-benzotriazole-1-yl)-1-(3-methoxyphenyl)ethanone and 0.56 mmol of 4-methoxybenzaldehyde are dissolved in 5 ml of ethanol. 0.28 mmol of piperidine is added to the reaction mixture and stirred at room temperature for 48 hours. The raw product is poured in water/ethyl acetate (1:1). The water phase is washed with ethyl acetate, the resulting organic phases are washed with a saturated sodium chloride solution, dried over magnesium sulfate and purified by column chromatography (hexane/ethyl acetate 9:1). Yield: 51%, yellow oil; Rf (hexane/ethyl acetate 5:5): 0.25; ¹H NMR (CDCl₃, 500 MHz): 8.04 (d, J=7.80 Hz, 1H, Harom), 7.75 (s, 1H, Harom), 7.31-7.29 (m, 3H, Harom), 7.21 (t, J=7.55 Hz, 1H, Harom), 7.19 (m, 2H, Harom), 7.00-6.95 (m, 1H, Harom), 6.66 (d, J=8.82 Hz, 2H, Harom), 6.58 (d, J=8.82 Hz, 2H, Harom), 3.66 (s, 3H, OMe9, 3.62 (s, 3H, OMe); ¹³C NMR (CDCl₃, 125 MHz): 189.95, 161.30, 158.65, 144.85, 141.60, 137.30, 132.30, 131.65, 131.65, 128.55, 127.35, 123.30, 120.45, 119.15 (2C), 118.05, 113.55 (2C), 112.40, 109.05; IR: 1655, 1595, 1259, 1175, 745 cm⁻¹.

### 48. 5-(3-Methoxyphenyl)-3-(4-methoxyphenyl)isoxazole

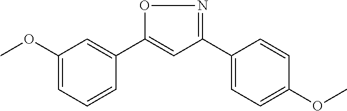

Synthesis: 0.28 mmol of 2-(1H-1,2,3-benzotriazole-1-yl)-1-4-(4-methoxyphenyl)-3-(3-methoxyphenyl)prop-2-ene-1-one and 0.56 mmol of hydroxylamine hydrochloride are stirred under reflux. After 18 hours, the reaction mixture is poured on a mixture of water/ethyl acetate (1:1). The aqueous phase is washed with ethyl acetate, the resulting organic phases are washed with a saturated sodium chloride solution, dried over magnesium sulfate and purified first by column chromatography (hexane/ethyl acetate 9:1) and then by preparative thin-layer chromatography (dichloromethane/methanol 1%); yield: 45%, yellow powder; Rf (dichloromethane/methanol 1%): 0.41; ¹H NMR (CDCl₃, 500 MHz): 7.20 (m, 2H, Harom), 6.75 (dd, J=7.50 Hz and J=2.00 Hz, 1H, Harom), 6.80 (d, J=7.50 Hz, 1H, Harom), 6.76 (s, 1H, Harom), 6.74 (s, 1H, Harom), 6.70 (m, 1H, Harom), 6.45 (d, J=7.50 Hz, 2H, Harom), 3.72 (s, 3H, OMe), 3.56 (s, 3H, OMe); ¹³C NMR (CDCl₃, 125 MHz): 171.65, 164.15, 162.40, 161.25, 131.80, 131.20, 128.70, 128.70, 121.60, 120.60, 117.35, 115.70, 113.00, 97.50; IR: 2925, 2853, 1602, 1248, 746 cm⁻¹.

### 49. 4,4′-Isoxazole-3,5-diyldiphenol (16)

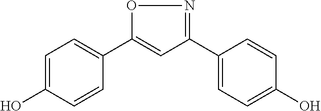

Synthesis: Prepared from 1.00 mmol of 3,5-bis(4-methoxyphenyl) isoxazole according to method E. Purification: column chromatography (hexane/ethyl acetate: 4:6); yield: 93%, yellow solid; Rf (dichloromethane/methanol 9:1): 0.73; ¹H NMR (CD₃OD, 500 MHz): 8.70-8.72 (m, 4H, Harom), 7.92 (s, 1H, Harom), 7.89 (m, 4H, Harom); ¹³C NMR (CD₃OD, 125 MHz): 172.10, 164.50, 161.10, 160.80, 132.60, 130.00, 129.50, 128.70, 121.70, 117.10, 116.90, 96.80; IR: 3321, 1610, 1509, 1443, 850 cm⁻¹; MS (ESI): (M+H)⁺: 254.

### 50. 3-[5-(4-Hydroxyphenyl)isoxazole-3-yl]phenol (17)

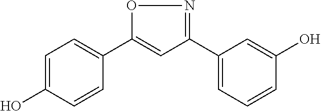

Synthesis: Prepared from 0.16 mmol of 3-(3-methoxyphenyl)-5-(4-methoxyphenyl)isoxazole according to method E. Purification: preparative thin-layer chromatography: (dichloromethane/methanol 5%). Yield: 36%, yellow powder; Rf (dichloromethane/methanol 9:1): 0.62; ¹H NMR (CD₃OD, 500 MHz): 7.89 (s, 1H, Harom), 7.42 (d, J=8.50 Hz, 2H, Harom), 7.20-7.17 (m, 3H, Harom), 6.93 (d, J=8.50 Hz, 2H, Harom), 6.77 (m, 1H, Harom); ¹³C NMR (CD₃OD, 125 MHz): 164.50, 162.00, 130.90, 128.20 (2C), 118.05, 116.65, 116.20, 115.55 (2C), 98.90; IR: 3369, 2905, 1652, 859 cm⁻¹; MS (ESI): (M+H)⁺: 254.

### 51. 3-[3-(4-Hydroxyphenyl)isoxazole-5-yl]phenol (18)

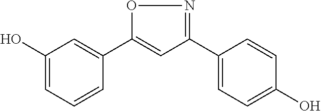

Synthesis: Prepared from 0.80 mmol of 5-(3-methoxyphenyl)-3-(4-methoxyphenyl)isoxazole according to method E. Purification: preparative thin-layer chromatography: (dichloromethane/methanol 5%). Yield: 36%, yellow powder; Rf (dichloromethane/methanol 9:1): 0.64; ¹H NMR (CD₃OD, 500 MHz): 7.85 (s, 1H, Harom), 7.32 (d, J=8.50 Hz, 2H, Harom), 7.10-7.07 (m, 3H, Harom), 7.05m, 1H, Harom), 6.90 (d, J=8.50 Hz, 2H, Harom); ¹³C NMR (CD₃OD, 125 MHz): 165.60, 164.90, 138.20, 126.20, 126.20, 118.05, 118.05, 112.60, 115.80, 115.70, 98.80; IR: 3409, 2905, 1652, 870 cm⁻¹; MS (ESI): (M+H)⁺: 254.

### 52. 5-Bromo-2-(3-methoxyphenyl)thiazole

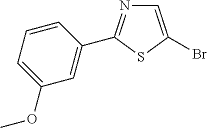

Synthesis: Prepared from 2.06 mmol of 2,5-dibromothiophene and 2.47 mmol of 3-methoxyphenylboronic acid according to method A, purification: column chromatography (dichloromethane/methanol 5%); yield: 50%, yellow solid; Rf (dichloromethane): 0.82; ¹H NMR (CDCl₃, 500 MHz): 7.45 (s, 1H, Harom), 7.16 (s.1H, Harom), 7.13 (dt, J=1.20 Hz and J=8.20, Hz, 1H, Harom), 7.05 (t, J=8.20 Hz, 8.20 Hz), 6.70 (m, 1H, Harom), 3.59 (s, 3H, OMe); ¹³C NMR (CDCl₃, 125 MHz): 159.00, 128.90, 123.10, 117.30, 112.00, 110.30, 54.30. IR: 2985, 1485, 992, 837 cm⁻¹.

### 53. 5-Bromo-2-(4-methoxyphenyl)thiazole

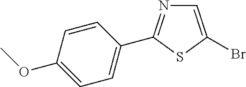

Synthesis: Prepared from 2.06 mmol of 2,5-dibromothiophene and 2.47 mmol of 4-methoxyphenylboronic acid according to method A, purification: column chromatography (dichloromethane/methanol 5%); yield: 50%, yellow solid; yield: 65%, yellow solid; Rf (hexane/ethyl acetate 7:3): 0.61; ¹H NMR (CDCl₃, 500 MHz): 7.52 (d, J=8.50 Hz, 2H, Harom), 7.39 (s, 1H, Hthiazole), 6.66 (d, J=8.50 Hz, 2H, Harem), 3.57 (s, 3H, OMe); ¹³C NMR (CDCl₃, 125 MHz): 168.35, 160.35, 142.95 (2C), 134.75, 127.00 (2C), 113.35, 54.35; IR: 1934, 2837, 1603, 1240, 1170, 827 cm⁻¹.

### 54. 5-(4-Methoxyphenyl)-2-(3-methoxyphenyl)thiazole

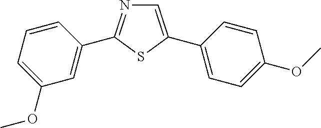

Synthesis: Prepared from 0.68 mmol of 5-bromo-2-(3-methoxyphenyl) thiazole and 1.37 mmol of 4-methoxyphenylboronic acid according to method A, purification: column chromatography (hexane/ethyl acetate:
9:1); yield: 58%, yellow oil; Rf (hexane/ethyl acetate 7:3): 0.58; ¹H NMR (CDCl₃, 500 MHz): 7.89 (s, 1H, Hthiazole), 7.51-7.49 (m, 4H, Harom), 7.32 (t, J=7.90 Hz, 1H, Harom), 6.93-6.91 (m, 3H, Harom), 3.86 (s, 3H, OMe), 3.82 (s, 3H, OMe); ¹³C NMR (CDCl₃, 125 MHz): 165.20, 159.05, 158.85, 138.35, 137.05, 128.95, 127.00 (2C), 117.95, 115.30, 113.55, 109.75, 54.75, 54.40; IR: 2980, 1580, 1240, 830 cm⁻¹.

### 55. 5-(3-Methoxyphenyl)-2-(4-methoxyphenyl)thiazole

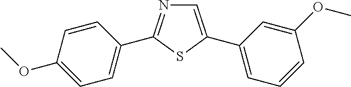

Synthesis: Prepared from 0.95 mmol of 5-bromo-2-(3-methoxyphenyl) thiazole and 1.37 mmol of 4-methoxyphenylboronic acid according to method A, purification: column chromatography (hexane/ethyl acetate: 9:1); yield: 50%, yellow oil; Rf (hexane/ethyl acetate 7:3): 0.60; ¹H NMR (CDCl₃, 500 MHz): 7.98 (s, 1H, Hthiazole), 7.53 (d, J=8.50 Hz, 2H, Harom), 7.51 (d, J=8.50 Hz, 2H, Harom), 7.33 (t, J=7.80 Hz, 1H, Harom), 7.19 (d, J=7.80 Hz, 1H, Harom), 7.11 (t, J=2.50 Hz, 1H, Harom), 6.85 (m, 1H, Harom), 3.87 (s, 3H, OMe), 3.84 (s, 3H, OMe); ¹³C NMR (CDCl₃, 125 MHz): 165.20; 159.05, 158.85, 138.35, 137.05, 128.95, 127.00 (2C), 117.95, 115.30, 113.55, 109.75, 54.75, 54.40; IR: δ 2978, 1602, 1238, 852 cm⁻¹.

### 56. 2,5-Bis-(4-methoxyphenyl)thiazole

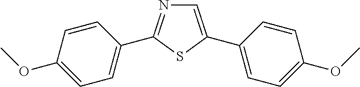

Synthesis: Prepared from 2.06 mmol of 2,5-dibromothiazole and 4.94 mmol of 4-methoxyphenylboronic acid according to method A, purification: column chromatography (dichloromethane/methanol 5%); yield: 10%, yellow solid; Rf (hexane/ethyl acetate 7:3): 0.45; ¹H NMR (CDCl₃, 500 MHz): 7.94 (s, 1H, Hthiazole), 7.74 (d, J=8.82 Hz, 2H, Harom), 6.95-6.90 (m, 4H, Harom), 3.83 (s, 3H, OMe), 3.82 (s, 3H, OMe); ¹³C NMR (CDCl₃, 125 MHz): 160.90, 158.90, 150.25, 137.00 (2C), 135.60, 127.25 (2C), 122.70, 113.80, 113.25, 112.45, 54.40, 54.15; IR: 2980, 1605, 1250, 837 cm⁻¹; MS (ESI): (M+H)⁺: 298.

### 57. 2,5-Bis(3-methoxyphenyl)thiazole

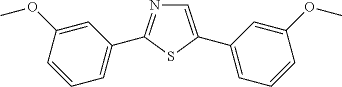

Synthesis: Prepared from 2.06 mmol of 2,5-dibromothiazole and 4.94 mmol of 4-methoxyphenylboronic acid according to method A, purification: column chromatography (hexane/ethyl acetate 9:1); yield: 40%, yellow oil; Rf (hexane/ethyl acetate 8:2): 0.38; ¹H NMR (CDCl₃, 500 MHz): 7.99 (s, 1H, Hthiazole), 7.55 (s, 1H, Harom), 7.51 (d, J=8.20 Hz. 1H, Harom), 7.34-7.31 (m, 2H, Harom), 7.17 (d, J=8.20 Hz, 1H, Harom), 7.10 (s, 1H, Harom), 6.97 (dd, J=8.20 Hz and J=2.50 Hz, 1H, Harom), 6.89 (J=8.20 Hz and J=2.50 Hz, 1H, Harom), 3.87 (s, 3H, OMe), 3.85 (s, 3H, OMe); ¹³C NMR (CDCl₃, 125 MHz): 160.10, 130.20, 130.05, 119.15, 116.80, 113.95, 112.40, 110.95, 55.50, 55.40; IR: 2984, 1608, 865 cm⁻¹,

### 58. 3-[5-(4-Hydroxyphenyl)-1,3-thiazole-2-yl]phenol (19)

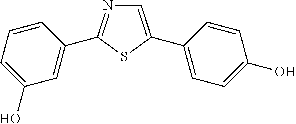

Synthesis: Prepared from 0.13 mmol of 5-(4-methoxyphenyl)-2-(3-methoxyphenyl)thiazole according to method E, purification: column chromatography (hexane/ethyl acetate 5:5); yield: 80%, yellow solid; Rf (hexane/ethyl acetate 5:5): 0.52; ¹H NMR (CD₃OD, 500 MHz): 7.80 (s, 1H, Hthiazole), 7.39 (d, J=8.80 Hz, 2H, Harom), 7.30 (m, 2H, Harom), 7.19 (t, J=8.20 Hz, 1H, Harom), 6.80-6.74 (m, 3H, Harom); ¹³C NMR (CD₃OD, 125 MHz): 167.70, 159.35, 159.25, 141.35, 138.25, 135.85, 131.25 (2C), 129.05, 123.60, 118.60, 118.30, 117.05 (2C), 113.80; IR: 3351, 2927, 1607, 1457, 830 cm⁻¹; MS (ESI): (M+H)⁺: 270.

### 59. 3-[2-(4-Hydroxyphenyl)-1,3-thiazole-5-yl]phenol (20)

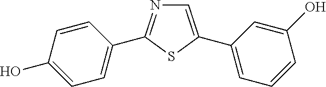

Synthesis: Prepared from 0.13 mmol of 5-(3-methoxyphenyl)-2-(4-methoxyphenyl)thiazole according to method E, purification: column chromatography (hexane/ethyl acetate 5:5); yield: 77%, yellow solid; Rf (H/E 5:5): 0.65. ¹H NMR (CD₃OD, 500 MHz): 8.01 (s, 1H, Hthiazole), 7.39 (m, 2H, Harom), 7.28 (m, 2H, Harom), 7.13 (d, J=7.80 Hz, 1H Harom), 7.07 (s, 1H, Harom), 6.88 (d, J=7.80 Hz, 1H, Harom), 6.78 (d, J=7.80 Hz, 1H, Harom); ¹³C NMR (CD₃OD, 125 MHz): 168.80, 159.30, 140.95, 139.70, 135.75, 133.45, 131.40 (2C), 131.20, 118.85, 118.85, 118.70, 116.70, 114.25, 113.90; IR: 3367, 2925, 2854, 1454, 1032, 750 cm⁻¹; MS (ESI): (M+H)⁺: 270.

60. 4,4′-(1,3-Thiazole-2,5-diyl)diphenol (21)

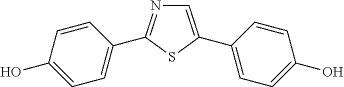

Synthesis: Prepared from 0.13 mmol of 5-(3-methoxyphenyl)-2-(4-methoxyphenyl)thiazole according to method E, purification: column chromatography (hexane/ethyl acetate 5:5); yield: 95%, yellow oil; Rf (hexane/ethyl acetate 5:5): 0.50; ¹H NMR (CD₃OD, 500 MHz): 7.73 (s, 1H, Hthiazole), 7.66 (d, J=8.80 Hz, 2H, Harom), 7.37 (d, J=8.80 Hz, 2H, Harom), 6.77-6.73 (m, 4H, Harom); ¹³C NMR (CD₃OD, 125 MHz): 168.25, 160.95, 159.20, 140.10, 137.85, 133.05, 129.95, 128.95, 128.90, 117.00, 116.90; IR: 3500, 1609, 1455, 836 cm⁻¹; MS (ESI): (M+H)⁺: 270.

### 61. 4,4′-(1,3-Thiazole-2,5-diyl)diphenol (22)

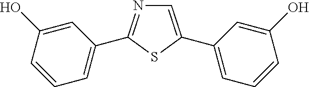

Synthesis: Prepared from 0.51 mmol of 5-(3-methoxyphenyl)-2-(4-methoxyphenyl)thiazole according to method E, purification: preparative thin-layer chromatography (hexane/ethyl acetate 5:5); yield: 85%, yellow oil; Rf (hexane/ethyl acetate 5:5): 0.42; ¹H NMR (CD₃COCD₃, 500 MHz): 8.62 (s, 2H, OH), 8.11 (s, 1H, Hthiazole), 7.52 (t, J=2.50 Hz, 1H, Harom), 7.48 (m, 1H, Harom), 7.34 (t, J=7.80 Hz, 1H, Harom), 7.28 (t, J=7.80 Hz, 1H, Harom), 7.19-7.16 (m, 2H, Harom), 6.97 (m, 1H, Harom), 6.87 (m, 1H, Harom); ¹³C NMR (CD₃COCD₃, 125 MHz): 158.95, 158.85, 140.35, 140.00, 135.90, 133.45, 131.25, 131.1.0, 118.75, 118.50, 118.15, 116.40, 114.15, 113.60; IR: 3517, 1695, 1453, 1242, 866 cm⁻¹; MS (ESI): (M+H)⁺: 270.

### 62. 4-Bromo-2-(3-methoxyphenyl)thiazole

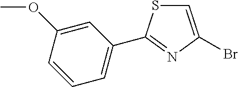

Synthesis: Prepared from 2.06 mmol of 2.4-dibromothiazole and 2.47 mmol of 3-methoxyphenylboronic acid according to method A, purification: column chromatography (hexane/ethyl acetate 9:1); yield: 50%, yellow oil; Rf (dichloromethane): 0.78; ¹H NMR (CDCl₃, 500 MHz): 7.50 (t, J=2.50 Hz, 1H, Harom), 7.48 (dt, J=7.80 Hz and J=2.50 Hz, 1H, Harom), 7.34 (t, J=7.80 Hz, 1H, Harom), 7.20 (s, 1H, Hthiazole), 7.00-6.98 (m, 1H, Harom), 3.86 (s, 3H, OMe); ¹³C NMR (CDCl₃, 125 MHz): 167.80, 159.00, 132.75, 129.00, 124.90, 117.80, 115.95, 115.55, 110.00, 54.45; IR: 3118, 2935, 2835, 1598, 1458, 1252, 1015, 782 cm⁻¹.

### 63. 4-Bromo-2-(4-methoxyphenyl)thiazole

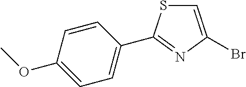

Synthesis: Prepared from 2.06 mmol of 2,4-dibromothiazole and 2.47 mmol of 4-methoxyphenylboronic add according to method A, purification: column chromatography (hexane/ethyl acetate 9:1); yield: 55%, yellow solid; Rf (dichloromethane): 0.78; NMR (CDCl₃, 500 MHz): 7.84 (d, J=8.80 Hz, 2H, Harom), 7.10 (s, 1H, Hthiazole), 6.91 (d, J=8.80 Hz, 2H, Harom), 3.82 (s, 3H, OMe); ¹³C NMR (CDCl₃, 125 MHz): 167.85, 161.00, 126.85, 124.65, 124.50, 114.35, 113.30, 54.40; IR: 3114, 1603, 1466, 1258, 825 cm⁻¹.

### 64. 4-(4-Methoxyphenyl)-2-(3-methoxyphenyl)thiazole

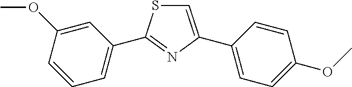

Synthesis: Prepared from 0.55 mmol of 4-bromo-2-(3-methoxyphenyl) thiazole and 0.77 mmol of 4-methoxyphenylboronic acid according to method A, purification: column chromatography (hexane/ethyl acetate 9:1); yield: 79%, white solid; Rf (hexane/ethyl acetate 9:1): 0.45; ¹H NMR (CDCl₃, 500 MHz): 7.93 (d, J=8.80 Hz, 2H, Harom), 7.62 (s, 1H, Harom), 7.61 (d, J=7.88 Hz, 1H, Harom), 7.36 (t, J=7.88 Hz, 1H, Harom), 7.30 (s, 1H, Hthiazole), 6.98-6.96 (m, 3H, Harom), 3.88 (s, 3H, Harom), 3.83 (s, 3H, OMe); ¹³C NMR (CDCl₃, 125 MHz): 166.45, 159.00, 158.65, 155.00, 134.10, 128.90, 127.00 (2C), 118.15, 115.00, 113.05 (2C), 110.45, 109.95, 54.40, 54.30; IR: 3108, 2961, 2837, 1596, 1481, 1249, 1173, 1036, 834 cm⁻¹.

### 65. 4-(3-Methoxyphenyl)-2-(4-methoxyphenyl)thiazole

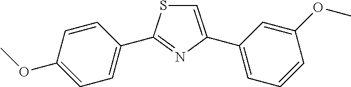

Synthesis: Prepared from 0.55 mmol of 4-bromo-2-(4-methoxyphenyl) thiazole and 0.77 mmol of 4-methoxyphenylboronic acid according to method A, purification: column chromatography (hexane/ethyl acetate 9:1); yield: 65%, yellow solid; Rf (hexane/ethyl acetate 5:5): 0.62; ¹H NMR (CDCl₃, 500 MHz): 7.97 (d, J=8.80 Hz, 2H, Harom), 7.59 (s.1H, Harom), 7.55 (dt, J=2.50 Hz and J=7.25 Hz. 1H, Harom), 7.37 (s, 1H, Harom), 7.34 (t, J=7.25 Hz, 1H, Harom), 6.95 (d, J=8.80 Hz, 2H, Harom), 6.90 (m, 1H, Harom), 3.87 (s, 3H, OMe), 3.83 (s, 3H, OMe); ¹³C NMR (CDCl₃, 125 MHz): 166.65, 160.15, 158.95, 154.80, 134.95, 128.95, 128.65 (2C), 127.05, 125.70, 117.85, 113.30 (2C), 112.00, 111.05, 54.35, 54.30; IR: 2954, 1596, 1250, 855 cm⁻¹.

### 66. 2,4-Bis(4-methoxyphenyl)thiazole

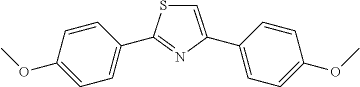

Synthesis, physical and chemical characterization already described by Fink, B. E., et al., Chem. and Biol., 6: 205-219 (1999).

### 67. 2,4-Bis(4-methoxyphenyl)thiazole

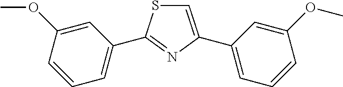

Synthesis: Prepared from 2.06 mmol of 2,4-dibromothiazole and 4.94 mmol of 3-methoxyphenylboronic acid according to method A, purification: column chromatography (hexane/ethyl acetate 9:1); yield: 18%, yellow oil; Rf (hexane/ethyl acetate 8:2): 0.40; ¹H NMR (CD₃COCD₃, 500 MHz): 7.92 (s, 1H, Hthiazole), 7.65-7.59 (m, 4H, Harom), 7.35 (t, J=7.90 Hz, 1H, Harom), 7.33 (t, J=7.90 Hz, 1H, Harom), 7.05 (m, 1H, Harom), 6.93 (m, 1H, Harom), 3.87 (s, 3H, OMe), 3.85 (s, 3H, OMe); ¹³C NMR (CD₃COCD₃, 125 MHz): 160.35, 160.25, 130.20, 129.75, 118.75, 118.65, 115.90, 113.65, 111.85, 111.40, 54.85, 54.70; IR: 3012, 2929, 1642, 1250, 812 cm⁻¹.

### 68. 3-[4-(4-Hydroxyphenyl)-1,3-thiazole-2-yl]phenol (23)

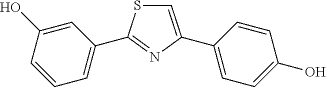

Synthesis: Prepared from 0.30 mmol of 4-(4-methoxyphenyl)-2-(3-methoxyphenyl)thiazole according to method E, purification: preparative thin-layer chromatography (hexane/ethyl acetate 5:5); yield: 80%, yellow oil; Rf (hexane/ethyl acetate 5:5): 0.45; ¹H NMR (CD₃OD, 500 MHz): 7.71 (d, J=8.80 Hz, 2H, Harom), 7.39 (s, 1H, Hthiophene), 7.36 (s, 1H, Harom), 7.34 (d, J=7.80 Hz, 1H, Harom), 7.17 (t, J=7.80 Hz, 1H, Harom), 6.76-6.74 (m, 3H, Harom); ¹³C NMR (CD₃OD, 125 MHz): 169.30, 159.15, 158.85, 157.70, 136.25, 131.20, 129.20, 128.95, 127.70, 118.90, 118.25, 116.75, 116.50, 114.10, 112.00; IR: 3671, 2988, 1609, 1480, 970, 836 cm⁻¹; MS (ESI): (M−H)⁺: 268.

### 69. 3-[2-(4-Hydroxyphenyl)-1,3-thiazole-4-yl]phenol (24)

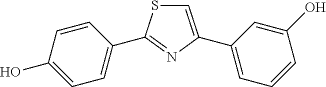

Synthesis: Prepared from 0.30 mmol of 4-(3-methoxyphenyl)-2-(4-methoxyphenyl)thiazole according to method E, purification: preparative thin-layer chromatography (hexane/ethyl acetate 5:5); yield: 78%, yellow oil; Rf (hexane/ethyl acetate 5:5): 0.52; ¹H NMR (CD₃COCD₃, 500 MHz): 8.87 (s, 1H, OH), 8.40 (s, 1H, OH), 7.93 (d, J=8.80 Hz, 2H, Harom), 7.91 (s, 1H, Hthiazole), 7.73 (s, 1H, Harom), 7.60 (d, J=7.80 Hz, 1H, Harom), 7.25 (t, J=7.80 Hz, 1H Harem), 6.96 (d, J=8.80 Hz, 2H, Harom), 6.83 (m, 1H, Harem); ¹³C NMR (CD₃COCD₃, 125 MHz): 170.95, 168.35, 160.35, 158.65, 156.55, 137.00, 130.55, 128.90, 126.60, 118.45, 116.70, 115.90, 114.20, 112.90; IR: 3351, 2962, 1689, 1587, 836 cm⁻¹; MS (ESI): (M−H)⁺: 268.

### 70. 4,4′-(1,3-Thiazole-2,4-diyl)diphenol (25)

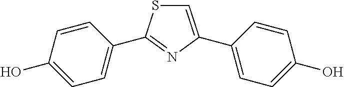

Synthesis, physical and chemical characterization already described by Fink, B. E., et al., Chem. and Biol., 6: 205-219 (1999).

### 71. 3,3′-(1,3-Thiazole-2,4-diyl)diphenol (26)

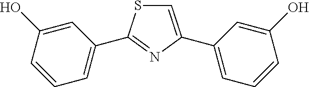

Synthesis: Prepared from 0.24 mmol of 2,4-bis(3-methoxyphenyl)thiazole according to method E, purification: preparative thin-layer chromatography (hexane/ethyl acetate 5:5); yield: 78%, yellow oil; Rf (hexane/ethyl acetate 5:5): 0.52; ¹H NMR (CD₃COCD₃, 500 MHz): 7.86 (s, 1H, Hthiazole), 7.59 (m, 2H, Harom), 7.54 (m, 2H, Harom), 7.35 (t, J=8.00 Hz, 1H, Harom), 7.29 (t, J=8.00 Hz, 1H, Harom), 6.97 (m, 1H, Harom), 6.85 (m, 1H, Harom); ¹³C NMR (CD₃COCD₃, 125 MHz): 159.85, 158.85, 135.95, 135.05, 130.20, 129.70, 117.70, 117.25, 115.10, 112.35; IR: 3312, 1635, 1622, 759 cm⁻¹; MS (ESI): (M−H)⁺: 268.

### 72. 2-Bromo-5-(4-methoxyphenyl)thiophene

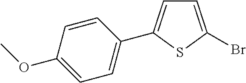

Synthesis: Prepared from 2.1 mmol of 2,5-dibromothiophene and 2.52 mmol of 4-methoxyphenylboronic acid according to method C, purification: column chromatography (hexane/ethyl acetate 9:1), yield: 75%, white powder; Rf (hexane/ethyl acetate 8:2): 0.72; ¹H NMR (CDCl₃, 500 MHz): 7.41 (d, J=8.80 Hz, 2H, Harom), 6.97 (d, J=3.78 Hz, 1H, Hthiophene), 6.91 (m, 3H, 2Harom+Hthiophene), 3.81 (s, 3H, OMe); ¹³C NMR (CDCl₃, 125 MHz): 158.50, 144.80, 129.70, 126.70 (2C), 121.15, 113.40, 109.15, 54.35; IR: 2955, 1606, 1501, 1252, 791 cm⁻¹.

### 73. 2-(3-Methoxyphenyl)-5-(4-methoxyphenyl)thiophene

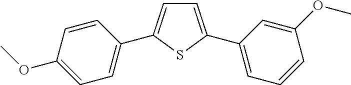

Synthesis: Prepared from 0.93 mmol of 2-bromo-5-(4-methoxyphenyl) thiophene and 1.11 mmol of 3-methoxyphenylboronic acid, purification: column chromatography (hexane/ethyl acetate 9:1), yield: 75%, yellow powder; Rf (hexane/ethyl acetate 8:2): 0.65; ¹H NMR (CDCl₃, 500 MHz): 7.47 (d, J=8.82 Hz, 2H, Harom), 7.20 (t, J=7.80 Hz, 1H, Harom), 7.17 (m, 1H, Hthiophene), 7.10 (m, 1H, Harom), 7.07 (m, 2H, Harom+Hthiophene), 6.84 (d, J=8.80 Hz, 2H, Harom), 6.76 (dd, J=7.80 Hz and J=2.50 Hz, 1H, Harom), 3.77 (s, 3H, OMe), 3.75 (s, 3H, OMe); ¹³C NMR (CDCl₃, 125 MHz): 158.95, 158.30, 142.70, 141.30, 134.75, 128.30, 126.20, 125.95, 123.15, 121.85, 118.70, 117.20, 113.35, 111.85, 110.20, 54.35, 54.30; IR: 2934, 1575, 1463, 1242, 1032, 811 cm⁻¹.

### 74. 2,5-Bis(4-methoxyphenyl)thiophene

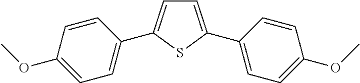

Synthesis: Prepared from 2.10 mmol of 2,5-dibromothiophene and 2.52 mmol of 4-methoxyphenylboronic acid according to method A, purification: column chromatography (hexane/ethyl acetate 9:1), yield: 10%, white powder; Rf (hexane/ethyl acetate 8:2): 0.62 ¹H NMR (CD₃COCD₃, 500 MHz): 7.15 (d, J=8.50 Hz, 4H, Harom), 6.90 (s, 2H, Hthiophene), 6.53 (d, J=8.50 Hz, 4H, Harom), 3.34 (s, 3H, OMe); ¹³C NMR (CD₃COCD₃, 125 MHz): 158.85, 126.40, 120.40, 114.50, 55.20; IR: 2854, 1598, 758 cm⁻¹.

### 75. 2,5-Bis(3-methoxyphenyl)thiophene

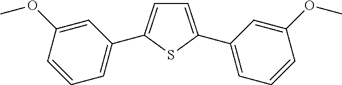

Synthesis: Prepared from 2.10 mmol of 2,5-dibromothiophene and 2.52 mmol of 3-methoxyphenylboronic acid according to method A, purification: column chromatography (hexane/ethyl acetate 9:1), yield: 8%, white powder; Rf (hexane/ethyl acetate 8:2): 0.57; ¹H NMR (CDCl₃, 500 MHz): 7.28 (t, J=7.80 Hz, 2H, Harom), 7.05 (s, 2H, Harom), 7.15 (m, 2H, Harom), 7.10 (m, 2H, Harom), 6.82 (s, 2H, Hthiophene), 3.83 (s, 6H, OMe); ¹³C NMR (CDCl₃, 125 MHz): 161.00, 142.30, 133.25, 129.15, 123.85, 120.00, 118.60, 109.45, 54.50; IR: 2930, 1600, 1242, 820 cm⁻¹.

### 76. 3-Bromo-2-(4-methoxyphenyl)thiophene

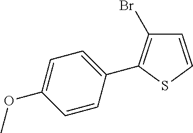

Synthesis: Prepared according to method A from 3.01 mmol of 2,3-dibromothiophene and 3.04 mmol of 4-methoxyphenylboronic acid, purification: column chromatography (hexane); yield: 70%, green solid; Rf (hexane-ethyl acetate. 9:1): 0.92; ¹H NMR (CD₃COCD₃, 500 MHz): 7.54 (d, J=9.20 Hz, 2H, Harom), 7.33 (d, J=1.30 Hz, 1H, Hthiophene), 7.22 (d, J=1.30 Hz, 1H, Hthiophene), 6.94 (d, J=9.20 Hz, 2H, Harom), 3.77 (s, 3H, OMe); ¹³C NMR (CD₃COCD₃, 125 MHz): 161.00, 127.80, 126.65, 125.40, 122.25, 115.40, 110.85, 55.75; IR: 2936, 1612, 1254, 852 cm⁻¹.

### 77. 3-Bromo-2-(4-methoxyphenyl)thiophene

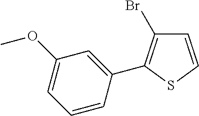

Synthesis: Prepared according to method C from 0.88 mmol of 2,3-dibromothiophene and 0.97 mmol of 3-methoxyphenylboronic acid, purification: column chromatography (Hexan); yield: 58%, yellow oil; Rf (hexane-ethyl acetate 9:1): 0.90; ¹H NMR (CDCl₃, 500 MHz): 7.32 (t, J=8.20 Hz, 1H, Harom), 7.26 (d, J=5.40 Hz, 1H, Hthiophene), 7.20 (m, 2H, Harom), 7.03 (d, J=5.40 Hz, 1H, Hthiophene), 6.91-6.89 (m, 1H, Harom), 3.83 (s, 3H, OMe); ¹³C NMR (CDCl₃, 125 MHz): 159.50, 138.10, 134.05, 131.70, 129.55, 125.70, 125.00, 121.50, 114.50, 114.05, 107.60, 55.35; IR: 3001, 2925, 1625, 1244, 869 cm⁻¹.

### 78. 2,3-Bis(4-methoxyphenyl)thiophene

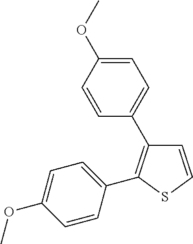

Synthesis: Prepared according to method A from 0.31 mmol of 3-bromo-2-(4-methoxyphenyl)thiophene and 0.34 mmol of 4-methoxyphenylboronic acid, purification: column chromatography (hexane); yield: 83%, yellow powder; ¹H NMR (CD₃COCD₃, 500 MHz): 7.41 (d, J=5.00 Hz, 1H, Hthiophene), 7.22-7.19 (m, 4H, Harom), 7.14 (d, J=5.00 Hz, 1H, Hthiophene), 6.87-6.84 (m, 4H, Harom), 3.84 (s, 3H, OMe), 3.79 (s, 3H, OMe); ¹³C NMR (CD₃COCD₃, 500 MHz): 158.70, 158.20, 130.55, 129.75, 129.70, 129.45, 128.40, 128.20, 126.85, 126.25, 113.60, 113.40, 113.20, 54.10, 54.05; IR: 2952, 1612, 1253, 752 cm⁻¹.

### 79. 3-(4-Methoxyphenyl)-2-(3-methoxyphenyl)thiophene

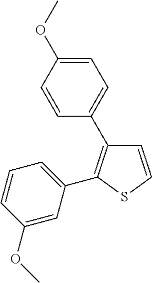

Synthesis: Prepared according to method C from 1.95 mmol of 3-bromo-2-(4-methoxyphenyl)thiophene and 2.34 mmol of 4-methoxyphenyl boronic acid, purification: column chromatography (hexane/ethyl acetate 7:3); yield 40%, white powder; Rf (hexane/ethyl acetate 7:3): 0.35; ¹H NMR (CDCl₃, 500 MHz): 7.22 (d, J=5.20 Hz, 1H, Hthiophene), 7.13 (d, J=8.50 Hz, 2H, Harom), 7.03 (d, J=5.20 Hz, 1H, Hthiophene), 7.10 (t, J=7.80 Hz, 1H, Harom), 6.77 (d, J=7.80 Hz, 1H, Harom), 6.76-6.74 (m, 3H, Harom), 6.70 (dd, J=2.50 Hz and J=7.80 Hz, 1H, Harom), 3.77 (s, 3H, OMe), 3.59 (s, 3H, OMe); ¹³C NMR (CDCl₃, 125 MHz): 158.40, 157.60, 136.85, 136.60, 134.80, 129.45, 129.20, 128.40, 128.05, 123.00, 120.70, 113.45, 112.80, 112.30, 54.20, 54.10; IR: 3011, 2836, 1605, 1252, 861 cm⁻¹.

### 80. 4,4′-Thiene-2,3-diyldiphenol (27)

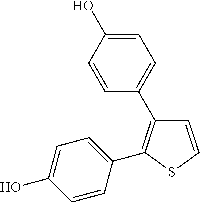

Synthesis: Prepared according to method E from 1.00 mmol of 2,3-bis(4-methoxyphenyl)thiophene, purification: column chromatography (hexane/ethyl acetate 5:5), yield: 70%, green powder; Rf (hexane/ethyl acetate 5:5): 0.49; ¹H NMR (CD₃OD, 500 MHz): 7.09-7.03 (m, 5H, Harom), 6.81 (d, J=5.50 Hz, 1H, Hthiophene), 6.69-6.65 (m, 4H, Harom); ¹³C NMR (CD₃OD, 125 MHz): 131.70, 131.55, 128.45, 123.95, 122.55, 119.10, 116.50, 116.25, 116.15, 116.10, 113.10, 108.80; MS (ESI): 269.

### 81. 3-[3-(4-Hydroxyphenyl)-2-thienyl]phenol (28)

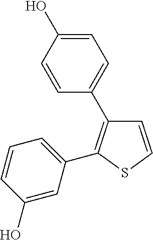

Synthesis: Prepared according to method E from 0.49 mmol of 3-(4-methoxyphenyl)-2-(3-methoxyphenyl)thiophene, purification: column chromatography (hexane/ethyl acetate 5:5); yield: 56%, green powder; Rf (H/E 5:5): 0.51; ¹H NMR (CD₃OD, 500 MHz): 7.35 (d, J=5.50 Hz, 1H, Harom), 7.09-7.06 (m, 4H, H), 6.75-6.72 (m, 5H, Harom); ¹³C NMR (CD₃OD, 125 MHz): 139.30, 131.90, 131.20, 130.50, 129.30, 124.80, 121.65, 117.10, 116.20, 115.35; IR: 3520, 2925, 1652, 825 cm⁻¹. MS (ESI): (M+H)⁺:269.

### 82. 3-[5-(4-Hydroxyphenyl)-2-thienyl]phenol (29)

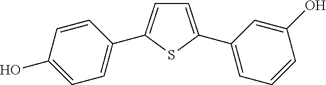

Synthesis: Prepared from 0.10 mmol of 2-(3-methoxyphenyl)-5-(4-methoxyphenyl)thiophene according to method E, purification: preparative thin-layer chromatography (hexane/ethyl acetate 5:5); yield: 93%, yellow powder; Rf (hexane/ethyl acetate 5:5): 0.48; ¹H NMR (CD₃COCD₃, 500 MHz): 8.57 (s, 1H, OH), 8.48 (s, 1H, OH), 7.53 (d, J=8.80 Hz, 2H, Harom), 7.33 (d, J=3.78 Hz, 1H, Hthiophene), 7.25-7.20 (m, 3H, 2Harom+Hthiophene), 7.15-7.13 (m, 2H, Harom), 6.89 (d, J=8.80 Hz, 2H, Harom), 6.78 (m, 1H, Harom); ¹³C NMR (CD₃COCD₃, 125 MHz): 157.90, 157.35, 143.70, 135.65, 130.05, 126.80, 124.20, 122.75, 116.60, 115.85, 114.50, 112.00; IR: 3301, 2967, 1242, 1033, 803 cm⁻¹; MS (ESI): (M+H)⁺:269.

### 83. 4,4′-Thiene-2,5-diyldiphenol (30)

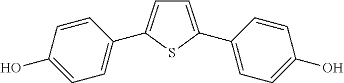

Synthesis: Prepared from 0.30 mmol of 2,5-bis(4-methoxyphenyl) thiophene according to method E, purification: preparative thin-layer chromatography (hexane/ethyl acetate 5:5); yield: 95%, yellow powder; Rf (hexane/ethyl acetate 5:5): 0.47; ¹H NMR (CD₃COCD₃, 500′MHz): 8.51 (s, 2H, OH), 7.50 (d, J=8.80 Hz, 4H, Harom), 7.21 (s, 2H, Hthiophene), 6.89 (d, J=8.80 Hz, 4H, Harom); ¹³C NMR (CD₃COCD₃, 125 MHz): 158.05, 143.20, 127.55, 127.05, 123.55, 116.70; IR: 3305, 1593, 798 cm⁻¹; MS (ESI): 269:(M+H)⁺.

### 84. 3,3′-Thiene-2,5-diyldiphenol (31)

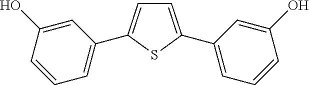

Synthesis: Prepared from 1.20 mmol of 2,5-bis(3-methoxyphenyl) thiophene according to method E, purification: preparative thin-layer chromatography (hexane/ethyl acetate 5:5); yield: 95%, yellow powder; Rf (hexane/ethyl acetate 5:5): 0.45; ¹H NMR (CD₃COCD₃, 500 MHz): 8.50 (s, 2H, —OHArom), 7.38 (s, 2H, Harom), 7.24 (t, J=7.80 Hz, 2H, Harom), 7.17 (m, 4H, Harom), 6.81 (m, 2H, Harom); ¹³C NMR (CD₃COCD₃, 125 MHz): 158.85, 144.10, 136.35, 131.00, 125.20, 117.65, 115.65, 113.05; IR: 3325, 2985, 1489, 852 cm⁻¹; MS (ESI): 269:(M+H)⁺.

### 85. 4-Bromo-2-(4-methoxyphenyl)thiophene

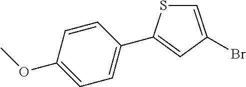

Synthesis: Prepared from 1.10 mmol of 2,4-dibromothiophene and 1.23 mmol of 4-methoxyphenylboronic acid according to method C, purification: column chromatography (hexane/ethyl acetate 9:1); yield: 78%, white powder; Rf (hexane/ethyl acetate 8:2): 0.79; ¹H NMR (CDCl₃, 500 MHz):7.46 (d, J=8.82 Hz, 2H, Harom), 7.08 (d, J=1.20 Hz, 1H, Hthiophene), 7.07 (d, J=1.20 Hz, 1H, Hthiophene), 6.90 (d, J=8.82 Hz, 2H, Harom), 3.81 (s, 3H, OMe); ¹³C NMR (CDCl₃, 125 MHz): 159.75, 145.40, 127.10 (2C), 126.05, 124.65, 120.90, 114.40, 110.35, 55.35; IR: 2937, 1606, 1291, 745 cm⁻¹.

### 86. 4-Bromo-2-(3-methoxyphenyl)thiophene

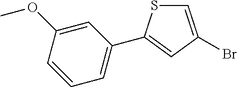

Synthesis: Prepared from 1.10 mmol of 2,4-dibromothiophene and 1.23 mmol of 3-methoxyphenylboronic acid according to method C, purification: column chromatography (hexane/ethyl acetate 9:1); yield: 72%, colorless oil; Rf (hexane/ethyl acetate 8:2): 0.80; ¹H NMR (CDCl₃, 500 MHz): 7.31 (t, J=7.80 Hz, 1H, Harom), 7.18 (d, J=2.00 Hz, 1H, Hthiophene), 7.15 (d, J=2.00 Hz, 1H, Hthiophene), 7.12 (m, 1H, Harom), 7.06 (m, 1H, Harom), 6.85 (dd, J=7.80 Hz and J=2.20 Hz, 1H, Harom), 3.83 (s, 3H, OMe); ¹³C NMR (CDCl₃, 125 MHz): 159.05, 144.30, 133.25, 129.05, 124.85, 121.00, 120.60, 117.70, 117.30, 112.75, 110.45, 109.45, 54.30; IR: 2970, 1650, 1252, 885, 770 cm⁻¹.

### 87. 4-(3-Methoxyphenyl)-2-(4-methoxyphenyl)thiophene

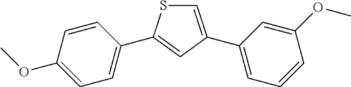

Synthesis: Prepared from 0.73 mmol of 4-bromo-2-(4-methoxyphenyl)thiophene and 0.88 mmol of 3-methoxyphenylboronic acid according to method C, purification: column chromatography (hexane/ethyl acetate 9:1); yield: 70%, slightly yellowish powder; Rf (hexane/ethyl acetate 8:2): 0.68; ¹H NMR (CD₃OD, 500 MHz): 7.51-7.48 (m, 3H, 2Harom+1Hthiophene), 7.38 (d, J=1.25 Hz, 1H, Hthiophene), 7.20 (t, J=7.80 Hz, 1H, Harom), 7.14 (m, 1H, Harom), 7.09 (m, 1H, Harom), 6.84 (d, J=8.80 Hz, 2H, Harom), 6.74 (m, 1H, Harom). ¹³C NMR (CD₃OD, 125 MHz): 173.00, 158.85, 158.55, 146.50, 144.30, 138.60, 130.85, 128.40, 128.10, 127.95, 127.45, 121.85, 119.25, 118.65, 116.75, 115.15, 114.00; IR: 3305, 2835, 1612, 750 cm⁻¹; MS (ESI): (M−H)⁺: 267.

### 88. 4-(4-Methoxyphenyl)-2-(3-methoxyphenyl)thiophene

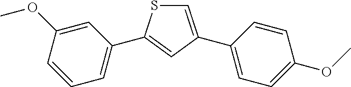

Synthesis: Prepared from 3.01 mmol of 4-bromo-2-(3-methoxyphenyl)thiophene and 3.60 mmol of 4-methoxyphenylboronic acid according to method C, purification: column chromatography (hexane/ethyl acetate 9:1); yield: 22%, slightly yellowish powder; Rf (hexane/ethyl acetate 7:3): 0.48; ¹H NMR (CDCl₃, 500 MHz): 7.42 (m, 3H, 2Harom+1Hthiophene), 7.19 (t, J=7.88 Hz, 1H, Harom), 7.13 (d, J=1.50 Hz, 1H, Hthiophene), 7.07 (m, 1H, Harom), 6.82 (d, J=8.50 Hz, 2H, Harom), 6.74 (m, 1H, Harom), 3.73 (s, 3H, OMe), 3.70 (s, 3H, OMe); ¹³C NMR (CDCl₃, 125 MHz): 159.00, 157.95, 143.70, 141.70, 134.90, 128.90, 127.65, 126.40, 121.40, 117.40, 113.15, 112.10, 110.50, 54.25, 54.20; IR: 2965, 1605, 1491, 1252, 1030, 826 cm⁻¹.

### 89. 2,4-Bis(3-methoxyphenyl)thiophene

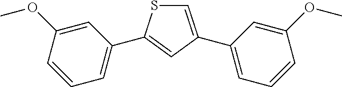

Synthesis: Prepared from 1.03 mmol of 4-bromo-2-(3-methoxyphenyl)thiophene and 3.66 mmol of 3-methoxyphenylboronic acid according to method C, purification: column chromatography (hexane/ethyl acetate 9:1); yield: 72%, slightly yellowish powder; Rf (hexane/ethyl acetate 8:2): 0.68; ¹H NMR (CDCl₃, 500 MHz): 7.47 (d, J=1.50 Hz, 1H, Hthiophene), 7.27 (d, J=1.50 Hz, 1H, Hthiophene), 7.20 (t, J=7.80 Hz, 2H, Harom), 7.15-7.12 (m, 2H, Harom), 7.13-7.10 (m, 2H, Harom), 6.77-6.75 (m, 2H, Harom), 3.76 (s, 6H, OMe); ¹³C NMR (CDCl₃, 125 MHz): 159.00, 143.85, 141.90, 136.25, 134.60, 128.95, 128.80, 121.55, 118.95, 117.85, 117.45, 112.20, 111.60, 111.15, 110.50, 54.30; IR: 2938, 1580, 1165, 777 cm⁻¹.

### 90. 3-[5-(4-Hydroxyphenyl)-3-thienyl]phenol (32)

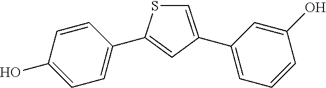

Synthesis: Prepared from 0.28 mmol of 4-(4-methoxyphenyl)-2-(3-methoxyphenyl)thiophene according to method E, purification: preparative thin-layer chromatography (hexane/ethyl acetate 5:5); yield: 80%, yellow powder; Rf (hexane/ethyl acetate 5:5): 0.48; ¹H NMR (CD₃OD, 500 MHz): 7.51-7.48 (m, 3H, 2Harom+1Hthiophene), 7.38 (d, J=1.20 Hz, 1H, Hthiophene), 7.20 (t, J=7.80 Hz, 1H, Harom), 7.14 (m, 1H, Harom), 7.09 (m, 1H, Harom), 6.84 (d, J=8.80 Hz, 2H, Harom), 6.74 (1H, Harom); ¹³C NMR (CD₃OD, 125 MHz): 173.00, 158.85, 158.55, 146.50, 14.30, 138.60, 130.85, 128.40, 128.10, 127.95, 127.45, 121.85, 119.25, 118.65, 116.75, 115.15, 114.00; MS (ESI): (M−H)⁺: 267.

### 91. 3-[4-(4-Hydroxyphenyl)-2-thienyl]phenol (33)

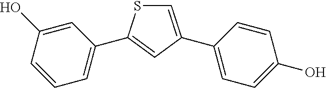

Synthesis: Prepared from 0.22 mmol of 4-(4-methoxyphenyl)-2-(3-methoxyphenyl)thiophene according to method E, purification: preparative thin-layer chromatography (hexane/ethyl acetate 5:5); yield: 85%, gray powder; Rf (hexane/ethyl acetate 5:5): 0.48; ¹H NMR (CD₃COCD₃, 500 MHz): 7.56 (d, J=1.50 Hz, 1H, Hthiophene), 7.45 (d, J=8.50 Hz, 2H, Harom), 7.30 (d, J=1.50 Hz, 1H, Hthiophene), 7.10 (t, J=7.80 Hz, 1H, Harom), 7.05 (m, 2H, Harom), 6.76 (d, J=8.50 Hz, 2H, Harom), 6.67 (m, 1H, Harom); ¹³C NMR (CD₃COCD₃, 125 MHz): 158.75, 157.75, 145.40, 143.95, 136.50, 130.90, 128.35, 128.25, 123.10, 118.55, 117.75, 116.45, 116.40, 115.60, 113.30; IR: 3502, 2985, 1601, 850 cm⁻¹; MS (ESI): (M−H)⁺: 267.

### 92. 3,3′-Thiene-2,4-diyldiphenol (34)

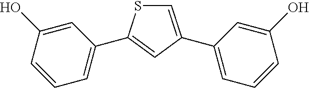

Synthesis: Prepared from 0.22 mmol of 2,4-bis(3-methoxyphenyl) thiophene according to method E, purification: preparative thin-layer chromatography (hexane/ethyl acetate 5:5); yield: 88%, yellow powder; Rf (hexane/ethyl acetate 5:5): 0.47; ¹H NMR (CD₃OD, 500 MHz): 7.61 (d, J=1.50 Hz, 1H, Hthiophene), 7.46 (d, J=1.50 Hz, 1H, Hthiophene), 7.20 (m, 2H, Harom), 7.14 (m, 2H, Harom), 7.09 (m, 2H, Harom), 6.73 (m, 2H, Harom); ¹³C NMR (CD₃OD, 125 MHz): 158.95, 158.80, 146.05, 144.30, 138.40, 136.85, 130.95, 130.80, 123.10, 120.40, 118.55, 117.95, 115.65, 115.15, 113.55, 113.30; IR: 3480, 2925, 1652, 855 cm⁻¹; MS (ESI): (M−H)⁺: 267.

### 93. 3-Methoxybenzoic acid-N-3-(methoxybenzoyl)hydrazide

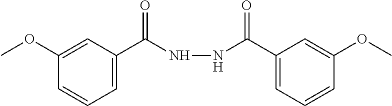

Synthesis: 11.77 mmol of benzoyl chloride is dissolved in 3 drops of DMF and cooled in an ice bath. 5.88 mmol of hydrazine monohydrate and 2 ml of triethylamine are added dropwise. After 30 min, the white precipitate is filtered off, washed with water and dried over night in a desiccator; yield; 90%, white solid; Rf: (hexane/ethyl acetate 1:9): 0.25; ¹H NMR (CD₃SOCD₃, 500 MHz): 10.31 (s, 2H, NH—CO), 7.53-7.42 (m, 6H, Harom), 7.16 (d, J=8.20 Hz, 2H, Harom), 3.85 (s, 6H, OMe); ¹³C NMR (CD₃SOCD₃, 125 MHz): 159.45, 148.25, 134.30, 129.60, 119.80, 117.75, 112.95, 55.50; IR: 3252, 1709, 1695, 1453, 866 cm⁻¹.

### 94. 2,5-Bis(3-methoxyphenyl)-1,3,4-oxadiazole

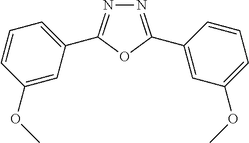

Synthesis: 1.74 mmol of 3-methoxybenzoic acid-N-3-(methoxybenzoyl) hydrazide, 2.09 mmol of Burgess reagent are dissolved in 10 ml of THF and heated under microwave conditions (100 W, 100° C.) for 10 minutes. After cooling to room temperature, the reaction mixture is washed with water, dried over magnesium sulfate, and the THF is removed on a rotary evaporator; yield: quantitative, white solid; Rf: (hexane/ethyl acetate 8:2): 0.65; ¹H NMR (CD₃COCD₃, 500 MHz): 7.75 (m, 2H, Harom), 7.69 (m, 2H, Harom), 7.52 (t, J=7.80 Hz, 2H, Harom), 7.20 (ddd, J=1.00 Hz and J=2.50 Hz and J=7.80 Hz, 2H, Harom), 3.93 (s, 3H, OMe); ¹³C NMR (CD₃COCD₃, 125 MHz): 165.25, 161.20, 131.35, 126.15, 119.90, 118.60, 112.65, 55.95; IR: 2920, 1515, 1254, 854 cm⁻¹.

### 95. 2,5-Bis(3-methoxyphenyl)-1,3,4-thiadiazole

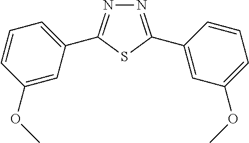

Synthesis: 0.83 mmol of 3-methoxybenzoic acid-N-3-(methoxybenzoyl) hydrazide and 1.67 mmol of Lawesson reagent are dissolved in 10 ml of THF and under microwave conditions (300 W, 90° C.) for 20 minutes. After cooling to room temperature, the reaction mixture is washed with water, dried over magnesium sulfate and purified by column chromatography (hexane/ethyl acetate: 8:2); yield: 70%, white-yellow solid; Rf: (hexane/ethyl acetate 7:3): 0.52; ¹H NMR (CD₃COCD₃, 500 MHz): 7.62-7.59 (m, 4H, Harom), 7.50 (t, J=8.20 Hz, 2H, Harom), 7.15 (dd, J=2.50 Hz and J=8.20 Hz, 2H, Harom), 3.92 (s, 6H, OMe); ¹³C NMR (CD₃COCD₃, 125 MHz): 168.65, 161.30, 131.40, 121.15, 118.00, 113.35, 55.90; IR: 2947, 1605, 1503, 1253, 835 cm⁻¹.

### 96. 3,3′-(1,3,4-Oxadiazole-2,5-diyl)diphenol (35)

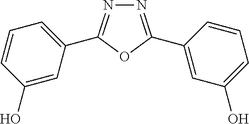

Synthesis: Prepared according to method E with 0.18 mmol of 2,5-bis(3-methoxyphenyl)-1,3,4-oxadiazole, purification: preparative thin-layer chromatography (hexane/ethyl acetate 5:5); yield: 92%, yellow solid; Rf: (hexane/ethyl acetate 5:5): 0.33; ¹H NMR (CD₃COCD₃, 500 MHz): 8.87 (s, 2H, OH), 7.64 (m, 4H, Harom), 7.44 (d, J=8.20 Hz, 2H, Harom), 7.11 (ddd, J=0.90 Hz and J=2.50 Hz and J=8.20 Hz, 2H, Harom); ¹³C NMR (CD₃COCD₃, 125 MHz): 165.22, 158.90, 131.40, 126.15, 119.85, 118.85, 114.20; IR: 3450, 2925, 1615, 752 cm⁻¹; MS (ESI): (M−H)⁺:253.

### 97. 3,3′-(1,3,4-Thiadiazole-2,5-diyl)diphenol (36)

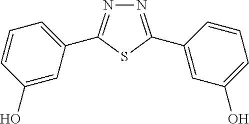

Synthesis: Prepared according to method E with 0.30 mmol of 2,5-bis(3-methoxyphenyl)-1,3,4-thiadiazole, purification: preparative thin-layer chromatography (hexane/ethyl acetate 5:5); yield: 82%, yellow solid; Rf: (hexane/ethyl acetate 5:5): 0.42; ¹H NMR (CD₃COCD₃, 500 MHz): 8.83 (s, 2H, OHarom), 7.58 (s, 2H, Harom), 7.50 (d, J=8.20 Hz, 2H, Harom), 7.39 (t, J=8.20 Hz, 2H, Harom), 7.05 (d, J=8.20 Hz, 2H, Harom); ¹³C NMR (CD₃COCD₃, 125 MHz): 158.95, 132.40, 131.45, 120.10, 119.25, 114.90; IR: 3399, 2854, 1612, 875 cm⁻¹; MS (ESI): (M−H)⁴: 269.

### 98. 3-Hydroxythiobenzamide

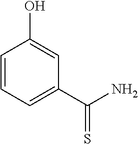

Synthesis: 4.19 mmol of 3-hydroxybenzonitrile, 4.19 mmol of a 50% ammonium sulfite solution and 5 ml of methanol are heated under microwave conditions (130° C., 130 W, 5 bar) for 30 minutes. After cooling to room temperature, the reaction mixture is washed with a saturated hydrogensulfite solution, dried over magnesium sulfate and evaporated. Yield: quantitative, orange oil; Rf: (hexane/ethyl acetate 6:4): 0.42; ¹H NMR (CD₃COCD₃, 500 MHz): 8.93 (s, 1H), 8.76 (s, 1H), 8.58 (s, 1H), 7.49 (s, 1H, Harom), 7.41 (d, J=8.20 Hz, 1H, Harom), 7.22 (t, J=8.20 Hz, 1H, Harom), 6.98 (dd, J=1.00 Hz and J=8.20 Hz, 1H, Harom); ¹³C NMR (CD₃COCD₃, 125 MHz): 156.95, 141.35, 128.95, 118.10, 117.85, 114.80; IR: 3500, 2924, 1633, 1380, 889 cm⁻¹.

### 99. 3,3′-(1,2,4-Thiadiazole-2,5-diyl)diphenol (37)

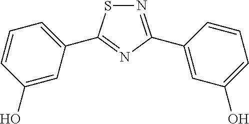

Synthesis: 0.17 mmol of 3-hydroxythiobenzamide and 3 ml of concentrated hydrochloric acid are stirred in 10 ml of DMSO at room temperature for 5 h. The reaction mixture is poured in 50 ml of water. The precipitate formed is filtered off, washed with water and dried over night in a desiccator. Yield: 92%, slightly yellowish solid; Rf: (hexane/ethyl acetate 5:5): 0.33; ¹H NMR (CD₃COCD₃, 500 MHz): 8.72 (s, 2H, OHarom), 7.87 (m, 2H, Harom), 7.59 (m, 2H, Harom), 7.45 (t, J=7.90 Hz, 1H, Harom), 7.38 (t, J=7.90 Hz, 1H, Harom), 7.11 (d, J=8.20 Hz, 1H, Harom), 7.02 (d, J=8.20 Hz, 1H, Harom); ¹³C NMR (CD₃COCD₃, 125 MHz): 158.20, 131.70, 130.70, 129.85, 119.50, 119.25, 118.75, 117.55, 114.85, 113.65; IR: 3396, 1610, 1445, 1286, 852 cm⁻¹; MS (ESI): (M−H)⁺: 269.

### 100. 3,5-Bis(4-methoxyphenyl)-1,2,4-thiadiazole

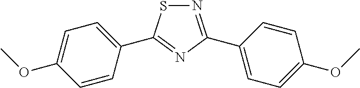

Synthesis: 1.90 mmol of 4-methoxyphenylthiobenzamide, 1.90 mmol of 3-hydroxythiobenzamide and 1.90 mmol of concentrated hydrochloric acid are stirred in 5 ml of DMSO at 35° C. for 8 hours. Thereafter, the reaction mixture is poured in water and the precipitate formed is filtered off, washed with water and dried over night in a desiccator. Purification: column chromatography (hexane/ethyl acetate 8:2); yield: 13%; Rf (hexane/ethyl acetate 8:2): 0.55; ¹H NMR (CD₃COCD₃, 500 MHz): 8.33 (d, J=8.80 Hz, 2H, Harom), 8.10 (d, J=8.80 Hz, 2H, Harom), 7.16 (d, J=8.80 Hz, 2H, Harom), 7.10 (d, J=8.80 Hz, 2H, Harom), 3.93 (s, 3H, OMe), 3.90 (s, 3H, OMe); ¹³C NMR (CD₃COCD₃, 125 MHz): 162.55, 130.60, 130.60, 130.05 (2C), 115.65 (2C), 114.90 (2C), 56.00, 55.75, IR: 2985, 1618, 1254, 854, 788 cm⁻¹.

### 101. 3-[3-(4-Methoxyphenyl)-1,2,4-thiadiazole-5-yl]phenol (38)

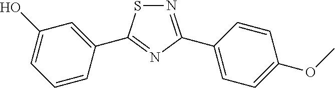

Synthesis: 1.90 mmol of 4-methoxyphenylthiobenzamide, 1.90 mmol of 3-hydroxythiobenzamide and 1.90 mmol of concentrated hydrochloric acid are stirred in 5 ml of DMSO at 35° C. for 8 hours. The reaction mixture is then poured in water, and the precipitate formed is filtered off, washed with water and dried over night in a desiccator. Purification: column chromatography (hexane/ethyl acetate 8:2); yield: 30%, yellow powder; Rf (hexane/ethyl acetate 8:2): 0.48; ¹H NMR (CD₃COCD₃, 500 MHz): 8.95 (s, 1H, OHarom), 8.33 (d, J=8.80 Hz, 2H, Harom), 7.88 (s.1H, Harom), 7.60 (d, J=7.60 Hz, 1H Harom), 7.44 (t, J=7.60 Hz, 1H, Harom), 7.16 (d, J=8.80 Hz, 2H, Harom), 7.11 (d, J=7.60 Hz, 1H, Harom), 3.89 (s, 3H, OMe); IR: 3452, 2932, 1632, 1242, 839 cm⁻¹.

### 102. 4,4′-(1,2,4-Thiadiazole-3,5-diyl)diphenol (39)

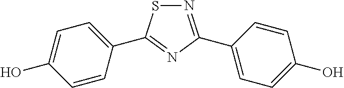

Synthesis: Prepared according to method E with 0.24 mmol of 3,5-bis(4-methoxyphenyl)-1,2,4-thiadiazole, purification: preparative thin-layer chromatography (hexane/ethyl acetate 5:5); yield: 92%, yellow solid; Rf (hexane/ethyl acetate 5:5): 0.33; ¹H NMR (CD₃COCD₃, 500 MHz): 9.20 (s, 1H, OHarom), 8.84 (s, 1H, OHarom), 8.24 (d, J=8.50 Hz, 2H, Harom), 8.00 (d, J=8.50 Hz, 2H Harom), 7.04 (d, J=8.50 Hz, 2H, Harom), 6.96 (d, J=8.50 Hz, 2H, Harom); ¹³C NMR (CD₃COCD₃, 125 MHz): 161.95, 130.80, 130.20, 125.95, 117.05, 116.35; IR: 3300, 1700, 1609, 837 cm⁻¹; MS (ESI): (M−H)⁺: 269.

### 103. 3-[3-(4-Hydroxyphenyl)-1,2,4-thiadiazole-5-yl]phenol (40)

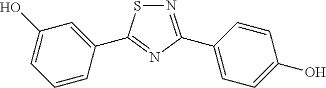

Synthesis: Prepared according to method E with 0.55 mmol of 3,5-bis(4-methoxyphenyl)-1,2,4-thiadiazole, purification: preparative thin-layer chromatography (hexane/ethyl acetate 5:5); yield: 91%, yellow oil; Rf (hexane/ethyl acetate 5:5): 0.35; ¹H NMR (CD₃COCD₃, 500 MHz): 8.86 (s, 1H, OHarom), 8.82 (s, 1H, OHarom), 8.23 (d, J=8.50 Hz, 2H, Harom), 7.57 (s, 1H, Harom), 7.54 (d, J=7.60 Hz, 1H, Harom), 7.38 (t, J=7.60 Hz, 1H, Harom), 7.07 (d, J=7.60 Hz, 1H, Harom), 6.98 (d, J=8.50 Hz, 2H, Harom); ¹³C NMR (CD₃COCD₃, 125 MHz): 187.85, 159.80, 158.15, 157.70, 131.85, 131.85, 130.65, 129.95, 124.85, 114.90 (2C); IR: 3310, 1695, 1609, 852 cm⁻¹; MS (ESI): (M−H)⁺: 269.

### 104. 3-Bromo-4′-methoxybiphenyl

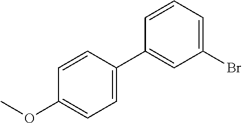

Synthesis: Prepared according to method A with 3.18 mmol of 1,3-dibromo-benzene and 3.50 mmol of 4-methoxyphenylboronic acid, purification: column chromatography (hexane/ethyl acetate 5%); yield: 50%, white solid; Rf (hexane/ethyl acetate 9:1): 0.48; ¹H NMR (CD₃COCD₃, 500 MHz): 7.76 (t, J=1.90 Hz, 0.1H, Harom), 7.59 (m, 3H, Harom), 7.46 (m, 1H, Harom), 7.36 (t, J=7.90 Hz, 1H, Harom), 7.01 (d, J=8.80 Hz, 2H, Harom), 3.83 (s, 3H, OMe); ¹³C NMR (CD₃COCD₃, 125 MHz): 160.85, 144.00, 132.50, 131.55, 130.30, 130.05, 128.95, 126.20, 115.30, 55.70; IR: 3035, 2933, 1610, 1517, 1248, 782 cm⁻¹.

### 105. 4,4″-Dimethoxy-[1,1′;3′,1″]terphenyl

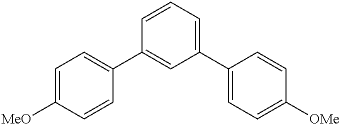

Synthesis: Prepared according to method A with 3.18 mmol of 1.3-dibromo-benzene and 3.50 mmol of 4-methoxyphenylboronic acid, purification: column chromatography (hexane/ethyl acetate 5%); yield: 12%, white solid; Rf: (hexane/ethyl acetate 9:1): 0.25; ¹H NMR (CDCl₃, 500 MHz): 7.69 (s, 1H, Harom), 7.56 (d, J=8.80 Hz, 4H, Harom), 7.46 (m, 3H, Harom), 6.97 (d, J=8.80 Hz, 4H, Harom), 3.84 (s, 6H, OMe); IR: 2957, 1607, 1517, 1249, 790 cm⁻¹.

### 106. 4′-Bromo-3-methoxybiphenyl

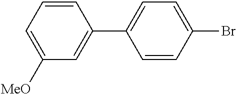

Synthesis: Prepared according to method B with 3.18 mmol of 1,4-dibromo-benzene and 3.50 mmol of 3-methoxyphenylboronic acid, purification: column chromatography (hexane/ethyl acetate 5%); yield: 35%, white solid; Rf: (hexane/ethyl acetate 9:1): 0.50; ¹H NMR (CDCl₃, 500 MHz): 7.56 (d, J=8.80 Hz, 2H, Harom), 7.45 (d, J=8.80 Hz, 2H, Harom), 7.35 (m, 1H, Harom), 7.14 (d, J=7.60 Hz, 1H, Harom), 7.09 (s, 1H, Harom), 6.92 (dd, J=2.50 Hz, J=8.20 Hz, 1H, Harom), 3.74 (s, 3H, OMe). IR: 3341, 2958, 1601, 1476, 1213, 777 cm⁻¹.

### 107. 3,3″-Dimethoxy-[1,1′;4′,1″]terphenyl

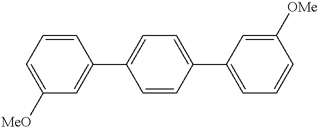

Synthesis: Prepared according to method B with 3.18 mmol of 1,4-dibromo-benzene and 3.50 mmol of 3-methoxyphenylboronic acid, purification: column chromatography (hexane/ethyl acetate 5%); yield: 14%, white solid; Rf: (hexane/ethyl acetate 9:1): 0.27; ¹H NMR (CD₃COCD₃, 500 MHz): 7.75 (s, 4H, Harom), 7.39 (t, J=7.90 Hz, 2H, Harom), 7.28 (d, J=7.90 Hz, 2H, Harom), 7.24 (s, 2H, Harom), 6.96 (dd, J=2.50 Hz, J=8.20 Hz, 2H, Harom), 3.88 (s, 6H, OMe); ¹³C NMR (CD₃COCD₃, 125 MHz): 130.80, 128.25, 119.95, 113.85, 113.20, 55.60; IR: 2925, 1581, 1479, 1221, 773 cm⁻¹.

### 108. 4,3″-Dimethoxy-[1,1′;3′,1″]terphenyl

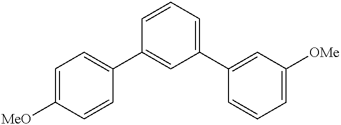

Synthesis: Prepared according to method A with 1.33 mmol of 1.3-bromo-4′-methoxybiphenyl and 1.46 mmol of 4-methoxyphenylboronic acid, purification: column chromatography (hexane/ethyl acetate 5%); yield: 56%, white solid; Rf: (hexane/ethyl acetate 9:1): 0.29; ¹H NMR (CDCl₃, 500 MHz): 7.66 (s, 1H, Harom), 7.49 (d, J=8.85 Hz, 2H, Harom), 7.43 (m, 2H, Harom), 7.37 (t, J=7.30 Hz, 1H, Harom), 7.26 (t, J=7.90 Hz, 1H, Harom), 7.13 (d, J=9.10 Hz, 1H, Harom), 7.08 (s, 1H, Harom), 6.90 (d, J=8.80 Hz, 2H, Harom), 6.81 (d, 1H, Harom), 3.76 (s, 3H, OMe), 3.74 (s, 3H, OMe); ¹³C NMR (CDCl₃, 125 MHz): 129.80, 129.15, 128.30, 125.90, 125.80, 125.65, 119.80, 114.30, 113.05, 112.80, 55.40, 55.35; IR: 2923, 1558, 1252, 888 cm⁻¹.

### 109. 4,3″-Dimethoxy-[1,1′;4′,1″]terphenyl

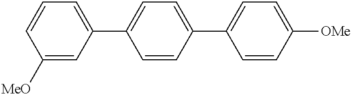

Synthesis: Prepared according to method B with 1.06 mmol of 4′-bromo-3-methoxybiphenyl and 2.33 mmol of 4-methoxyphenylboronic acid, purification: column chromatography (hexane/ethyl acetate 5%); yield: 90%, white solid; Rf: (hexane/ethyl acetate 9:1): 0.29; ¹H NMR (CDCl₃, 500 MHz): 7.62 (m, 4H, Harom), 7.56 (d, J=8.80 Hz, 2H, Harom), 7.35 (t, J=7.90 Hz, 1H, Harom), 7.21 (d, J=7.60 Hz, 1H, Harom), 7.15 (t, J=1.90 Hz, 1H, Harom), 6.98 (d, J=8.80 Hz, 2H, Harom), 6.88 (dd, J=2.50 Hz, J=8.20 Hz, 1H, Harom), 3.10 (s, 3H, OMe), 3.79 (s, 3H, OMe); ¹³C NMR (CDCl₃, 125 MHz): 129.80, 128.05, 127.50, 127.00, 119.55, 114.30, 112.65, 55.35, 55.30; IR: 2975, 1685, 1259, 850 cm⁻¹.

### 110. [1,1′;3′,1″]Terphenyl-4,4″-diol (41)

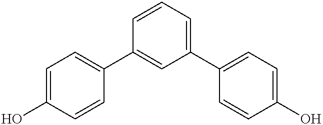

Synthesis: Prepared according to method. E with 0.24 mmol of 4,4″-dimethoxy-[1,1′;3′,1″]terphenyl, purification: column chromatography (hexane/ethyl acetate 7:3); yield: 90%, yellow powder; Rf: (H/E 5:5): 0.52; ¹H NMR (CD₃OD, 500 MHz): 7.66 (t, 1H, Harom), 7.94 (d, J=8.80 Hz, 4H, Harom), 7.39 (m, 3H, Harom), 7.86 (d, J=8.50 Hz, 4H, Harom); ¹³C NMR (CD₃OD, 125 MHz): 130.10, 129.20, 125.65, 116.65; IR: 3485, 2989, 1609, 1517, 1238, 790 cm⁻¹; MS (ESI): 261: (M−H)⁺.

### 111. [1,1′,4′,1″]Terphenyl-3,3′-diol (42)

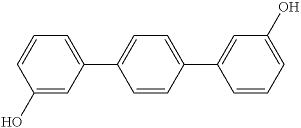

Synthesis: Prepared according to method E with 0.24 mmol of 3,3″-dimethoxy-[1,1′;4′,1″]terphenyl, purification: column chromatography (hexane/ethyl acetate 7:3); yield: 90%, yellow powder; Rf: (H/E 5:5): 0.53; ¹H NMR (CD₃OD, 500 MHz): 7.64 (s, 4H, Harom), 7.25 (t, J=8.20 Hz, 2H, Harom), 7.12 (d, J=7.60 Hz, 2H, Harom), 7.07 (t, J=2.20 Hz, 2H, Harom), 6.77 (d, J=7.90 Hz, 2H, Harom); ¹³C NMR (CD₃OD, 125 MHz): 130.90, 128.25, 119.20, 115.35, 114.65; IR: 3371, 2974, 1406, 1250, 1046, 780 cm⁻¹; MS (ESI): 261: (M−H)⁺.

### 112. [1,1′;3′,1″]Terphenyl-4,3″-diol (43)

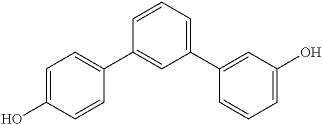

Synthesis: Prepared according to method E with 0.52 mmol of 4,3″-dimethoxy-[1,1′;3′,1″]terphenyl, purification: column chromatography (hexane/ethyl acetate 7:3); yield: 97%, yellow powder; Rf: (H/E 5:5): 0.52; ¹H NMR (CD₃COCD₃, 500 MHz): 8.45 (s, 1H, OHarom), 8.41 (s, 1H, OHarom), 7.78 (s, 1H, Harom), 7.54 (m, 4H, Harom), 7.44 (t, J=7.60 Hz, 1H, Harom), 7.27 (t, J=8.20 Hz, 1H, Harom), 7.17 (d, J=8.20 Hz, 2H, Harom), 6.94 (d, J=8.50 Hz, 2H, Harom), 6.85 (m, 1H, Harom); ¹³C NMR (CD₃COCD₃, 125 MHz): 129.90, 129.20, 128.15, 125.35, 125.00, 118.30, 115.75, 114.40, 113.90; IR: 3361, 1593, 1463, 1241, 776 cm⁻¹; MS (ESI): 261: (M−H)⁴.

### 113. [1,1′;4′,1″]Terphenyl-4,3″-diol (44)

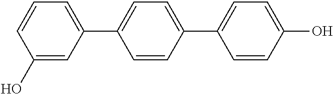

Synthesis: Prepared according to method E with 0.52 mmol of 4,3″-dimethoxy-[1,1′;4′,1″]terphenyl, purification: column chromatography (hexane/ethyl acetate 7:3); yield: 97%, yellow powder; Rf: (H/E 5:5): 0.52; ¹H NMR (CD₃COCD₃, 500 MHz): 8.42 (s, 1H, OHarom), 8.37 (s, 1H, OHarom), 7.62 (s, 4H, Harom), 7.51 (d, J=8.50 Hz, 2H, Harom), 7.22 (t, J=8.20 Hz, 1H, Harom), 7.11 (m, 2H, Harom), 6.89 (d, J=8.50 Hz, 2H, Harom), 6.70 (d, J=8.20 Hz, 1H, Harom); ¹³C NMR (CD₃COCD₃, 125 MHz): 130.75, 128.75, 128.05, 127.50, 118.80, 116.65, 115.15, 114.40; IR: 3220, 1590, 1454, 1202, 780 cm⁻¹; MS (ESI): 261: (M−H)⁺.

### 114. 4-[5-(3-Hydroxyphenyl)-2-thienyl]-2-methylphenol (45)

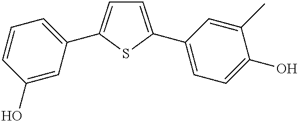

Synthesis: Suzuki cross coupling reaction (method A) followed by ether cleavage with boron tribromide (method E). Rf (hexane/ethyl acetate 1:1): 0.42; ¹H NMR (CD₃COCD₃, 500 MHz): 8.25 (s, 1H, OH), 8.23 (s, 1H, OH), 7.44 (d, J=1.50 Hz, 1H, arom. H), 7.34 (m, 2H, arom. H), 7.25-7.22 (m, 2H, arom. H), 7.14 (m, 2H, arom. H), 6.86 (d, J=8.20 Hz, 1H, arom. H), 6.78 (dd, J=1.50 Hz and 8.20 Hz, 1H, arom. H), 2.25 (s, 3H, CH₃); ¹³C NMR (CD₃COCD₃, 125 MHz): 149.15, 140.90, 135.25, 131.05, 131.05, 129.35, 129.30, 127.75, 121.75, 120.35, 119.65, 117.15, 20.50; IR (neat): 3514, 2928, 2853, 1598, 798 cm⁻¹; MS (ESI): 281 (M−H)⁺.

### 115. 4-[5-(3-Hydroxyphenyl)-2-thienyl]benzene-1,2-diol (46)

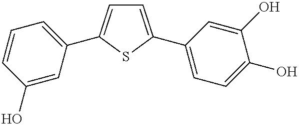

Synthesis: Suzuki cross coupling reaction (method A) followed by ether cleavage with boron tribromide (method E). Rf (hexane/ethyl acetate 1:1): 0.12; ¹H NMR (CD₃COCD₃, 500 MHz): 7.33 (d, J=3.80 Hz, 1H, thiophene H), 7.21 (m, 2H, arom. H), 7.17 (d, J=2.20 Hz, 1H, arom. H), 7.14 (m, 2H, arom. H), 7.06 (dd, J=8.20 Hz and J=2.20 Hz, 1H, arom. H), 6.88 (d, J=8.20 Hz, 1H, arom. H), 6.79 (m, 1H, arom. H); ¹³C NMR (CD₃COCD₃, 125 MHz): 146.40, 146.20, 144.75, 143.00, 130.95, 125.05, 123.65, 118.30, 117.50, 116.70, 115.35, 113.50, 112.90; IR (neat): 3319, 2989, 2901, 1581, 1221, 774 cm⁻¹; MS (ESI): 283 (M−H)⁺.

### 116. 2-Fluoro-4-[5-(3-hydroxyphenyl)-2-thienyl]phenol (47)

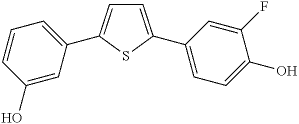

Synthesis: Suzuki cross coupling reaction (method A) followed by ether cleavage with boron tribromide (method E). Rf (hexane/ethyl acetate 1:1): 0.48; ¹H NMR (CD₃COCD₃, 500 MHz): 8.86 (s, 1H, OH), 8.51 (s, 1H, OH), 7.44 (d, J=12.20 Hz, 1H, arom. H), 7.36-7.32 (m, 3H, arom. H), 7.23 (t, J=8.80 Hz, 1H, arom. H), 7.15 (m, 2H, arom. H), 7.07 (t, J=8.80 Hz, 1H, arom. H), 6.95 (d, J=7.90 Hz, 1H, arom. H); ¹³C NMR (CD₃COCD₃, 125 MHz): 158.90, 153.50, 151.60, 145.60, 145.45, 143.60, 143.10, 143.05; 136.35, 131.00, 127.70, 127.65, 125.20, 124.65, 122.80, 122.75, 119.25, 119.20, 117.60, 115.60, 114.00, 113.80, 113.00; IR (neat): 3332, 1582, 1550, 780 cm⁻¹; MS (ESI): 285 (M−H)⁺.

### 117. 2,6-Difluoro-4-[5-(3-hydroxyphenyl)-2-thienyl]phenol (48)

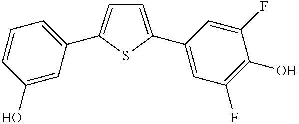

Synthesis: Suzuki cross coupling reaction (method A) followed by ether cleavage with boron tribromide (method E). Rf (hexane/ethyl acetate. 1:1): 0.41; ¹H NMR (CD₃COCD₃, 500 MHz): 7.39 (d, J=3.80 Hz, 1H, thiophene H), 7.37 (d, J=3.80 Hz, 1H, thiophene H), 7.30 (d, J=1.50 Hz, 1H, arom. H), 7.28 (d, J=1.50 Hz, arom. H), 7.22 (d, J=8.00 Hz, 1H, arom. H), 7.13 (m, 2H, arom. H), 6.79 (m, 1H, arom. H); ¹³C NMR (CD₃COCD₃, 125 MHz): 157.95, 143.55, 135.25, 130.20, 124.80, 124.45, 120.00, 116.80, 114.90, 112.20, 108.85, 108.80, 108.70, 108.65; IR (neat): 3436, 2962, 1583, 1487, 1244, 772 cm⁻¹; MS (ESI): 303 (M−H)⁺.

### 118. 4-[5-(3-Hydroxyphenyl)-2-thienyl]-2-(trifluoromethyl)phenol (49)

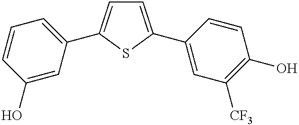

Synthesis: Suzuki cross coupling reaction (method A) followed by ether cleavage with boron tribromide (method E). Rf (hexane/ethyl acetate 1:1): 0.47; ¹H NMR (CD₃COCD₃, 500 MHz): 7.71 (d, J=2.30 Hz, 1H, arom. H), 7.66 (dd, J=8.50 Hz and J=J=2.30 Hz, 1H, arom. H), 729 (m, 2H, arom. H), 7.14 (t, J=7.90 Hz, 1H, arom. H), 7.06-7.03 (m, 3H, arom. H), 6.67 (m, 1H, arom. H); ¹³C NMR (CD₃COCD₃, 125 MHz): 158.90, 143.90, 136.70, 131.40, 131.0, 125.30, 124.90, 118.70, 117.60, 115.70, 113.05; IR (neat): 3491, 3387, 1583, 1490, 799 cm⁻¹; MS (ESI): 285 (M−H)⁺.

### 120. 3-[5-(3-Fluorophenyl)-2-thienyl]phenol (50)

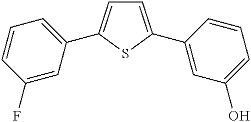

Synthesis: Suzuki cross coupling reaction (method A) followed by ether cleavage with boron tribromide (method E). Rf (hexane/ethyl acetate 6:4): 0.52; ¹H NMR (CD₃COCD₃, 500 MHz): 8.20 (s, 1H, OH), 7.39 (m, 2H, arom. H), 7.34-7.29 (m, 3H, arom. H), 7.13 (t, J=7.90 Hz, 1H, arom. H), 7.06 (s, 1H, arom. H), 7.04 (s, 1H, arom. H), 6.95 (m, 1H, arom. 1H), 6.71 (m, 1H, arom. H); ¹³C NMR (CD₃COCD₃, 125 MHz): 163.10, 158.85, 145.15, 142.30, 137.40, 137.35, 136.10, 131.90, 131.10, 126.30, 125.40, 122.20, 117.70, 115.90, 115.05, 114.90, 113.15, 112.75, 112.60; IR (neat): 2989, 2901, 1580, 1242, 1057, 775 cm⁻¹; MS (ESI): 269 (M−H)⁺.

### 121. N-{3-[5-(3-Hydroxyphenyl)-2-thienyl]phenyl}methanesulfonamide (51)

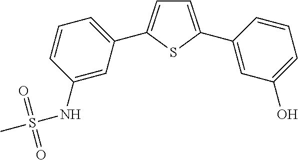

Synthesis: Suzuki cross coupling reaction (method A) followed by ether cleavage with boron tribromide (method E). Rf (hexane/ethyl acetate 4:6): 0.42; ¹H NMR (CD₃COCD₃, 500 MHz): 8.68 (s, 1H), 8.53 (s, 1H), 7.67 (s, 1H, arom. H), 7.47 (d, J=8.20 Hz, 1H, arom. H), 7.42 (m, 3H, arom. H), 7.31 (d, J=8.80 Hz, 1H, arom. H), 7.25 (t, J=7.90 Hz, 1H, arom. H), 7.17 (m, 2H, arom. H), 6.83 (d, J=8.20 Hz, 1H, arom. H) 3.05 (s, 3H, CH₃); ¹³C NMR (CD₃COCD₃, 125 MHz): 160.00, 143.60, 142.65, 135.30, 130.15, 130.10, 129.30, 124.85, 124.75, 124.30, 121.20, 120.20, 119.15, 117.85, 116.85, 112.95, 110.80, 32.00; IR (neat): 3279, 1587, 1470, 1142, 783 cm⁻¹; MS (ESI): 344 (M−H)⁺.

### 122. 3-(5-Phenyl-2-thienyl)phenol (52)

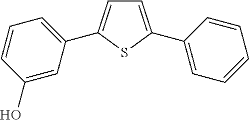

Synthesis: Suzuki cross coupling reaction (method A) followed by ether cleavage with boron tribromide (method E). Rf (hexane/ethyl acetate 7:3): 0.62; ¹H NMR (CD₃COCD₃, 500 MHz): 8.51 (s, 1H, OH), 7.70 (d, J=8.50 Hz, 2H, arom. H), 7.44-7.42 (m, 4H, arom. H), 7.30 (t, J=7.20 Hz, 1H, arom. H), 7.19 (t, J=7.25 Hz, 1H, arom. H), 7.18 (m, 2H, arom. H), 6.80 (d, J=7.80 Hz, 1H, arom. H); ¹³C NMR (CD₃COCD₃, 125 MHz): 158.85, 114.30, 144.00, 136.35, 135.05, 131.05, 129.95, 129.95, 128.50, 126.25, 126.25, 125.30, 125.25, 117.65, 115.70, 113.05; IR (neat): 3416, 1582, 1442, 1180, 752 cm⁻¹; MS (ESI): 351 (M−H)⁺.

### 123. 3-[5-(4-Hydroxyphenyl)-2-thienyl]-5-methylphenol (53)

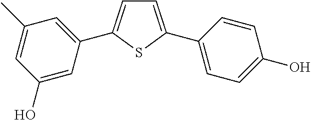

Synthesis: Suzuki cross coupling reaction (method A) followed by ether cleavage with boron tribromide (method E). Rf (hexane/ethyl acetate 1:1): 0.42; ¹H NMR (CD₃COCD₃, 500 MHz): 8.57 (s, 1H, OH), 8.36 (s, 1H, OH), 7.51 (d, J=8.50 Hz, 2H, arom. H), 7.29 (d, J=3.60 Hz, 1H, thiophene H), 7.21 (d, J=3.60 Hz, 1H, thiophene. H), 6.96 (s, 1H, arom. H), 6.92 (s, 1H, arom. H), 6.88 (d, J=8.50 Hz, 2H, arom. H), 6.60 (s, 1H, arom. H), 2.26 (s, 3H, CH₃aliphatic); ¹³C NMR (CD₃COCD₃, 125 MHz): 157.85, 157.35, 143.45, 142.05, 139.95, 135.40, 126.85, 126.80, 125.95, 124.05, 122.65, 117.45, 115.85, 115.25, 109.30, 20.55; IR (neat): 3308, 2948, 1593, 1220, 827 cm⁻¹; MS (ESI): 281 (M−H)⁺.

### 124. 3-[5-(4-Fluorophenyl)-2-thienyl]phenol (54)

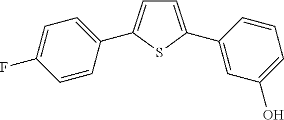

Synthesis: Suzuki cross coupling reaction (method A) followed by ether cleavage with boron tribromide (method E). Rf (hexane/ethyl acetate 1:1): 0.74; ¹H NMR (CD₃COCD₃, 500 MHz): 8.48 (s, 1H, OH), 7.75-7.72 (m, 2H, arom. H), 7.40 (m, 2H, arom. H), 7.25-7.16 (m, 5H, arom. H), 6.81 (m, 1H, arom. H); ¹³C NMR (CD₃COCD₃, 125 MHz): 135.50, 133.15, 127.15, 126.55, 120.10, 117.45; IR (neat): 3482, 2925, 1585, 799 cm⁻¹; MS (ESI): 269 (M−H)⁺.

### 125. 4-[5-(3-Hydroxyphenyl)-3-thienyl]-2-methylphenol (55)

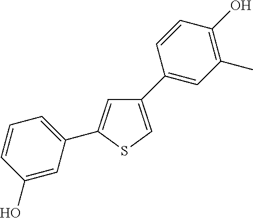

Synthesis: Suzuki cross coupling reaction (method A) followed by ether cleavage with boron tribromide (method E). Rf (hexane/ethyl acetate 1:1): 0.42; ¹H NMR (CD₃COCD₃, 500 MHz): 8.49 (s, 1H, OH), 8.30 (s, 1H, OH), 7.73 (s, 1H, arom. H), 7.52 (s, 1H, arom. H), 7.46 (s, 1H, arom. H), 7.42 (d, J=8.20 Hz, 1H, arom. H), 7.22-7.19 (m, 3H, arom. H), 6.87 (d, J=8.20 Hz, 1H, arom. H), 6.81 (m, 1H, arom. H), 2.25 (s, 3H, CH₃); ¹³C NMR (CD₃COCD₃, 125 MHz): 170.95, 158.80, 155.85, 145.25, 144.10, 136.70, 130.95, 129.65, 128.30, 125.50, 125.40, 123.20, 118.40, 117.75, 115.85, 115.55, 113.24, 16.30; IR (neat): 3288, 2916, 1600, 782 cm⁻¹; MS (ESI): 281 (M−H)⁺.

### 126. 4-[2-(3-Hydroxyphenyl)-1,3-thiazole-5-yl]-2-methylphenol (56)

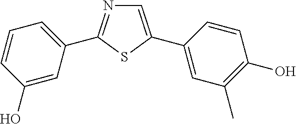

Synthesis: Suzuki cross coupling reaction (method A) followed by ether cleavage with boron tribromide (method E). Rf (hexane/ethyl acetate 1:1): 0.55; ¹H NMR (CD₃COCD₃, 500 MHz): 7.97 (s, 1H, arom. H), 7.49 (s, 1H, arom. H), 7.45 (m, 2H, arom. H), 7.32 (m, 1H, arom. H), 7.30 (t, J=8.20 Hz, 1H arom. H), 6.90 (m, 2H, arom. H), 2.25 (s, 3H, CH₃); ¹³C NMR (CD₃COCD₃, 125 MHz): 170.95, 158.80, 155.85, 145.25, 144.10, 136.70, 130.95, 129.65, 128.30, 125.50, 125.10, 123.20, 118.40, 117.75, 115.85, 115.55, 113.24, 16.30; IR (neat): 3300, 2906, 1572, 1222, 817 cm⁻¹; MS (ESI): 281 (M−H).

### 127. 3,3′-Pyridine-2,5-diyldiphenol (57)

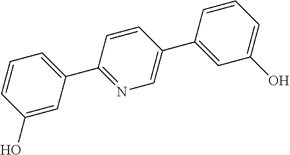

Synthesis: Suzuki cross coupling reaction (method A) followed by ether cleavage with boron tribromide (method E). Rf (hexane/ethyl acetate 1:1): 0.46; ¹H-NMR (500 MHz, CD₃COCD₃): 8.91 (dd, J=0.90 Hz, J=2.5 Hz, 1H, arom. H) 8.63 (s, 2H, OH), 8.04 (dd, J=8.20 Hz, J=2.20 Hz, 1H, arom. H), 7.92 (d, J=8.20 Hz, 1H, arom. H), 7.71 (t, J=2.50 Hz, 1H, arom. H), 7.61 (d, J=7.60 Hz, 1H, arom. H), 7.30-7.35 (m, 2H, Arom. H), 7.20-7.21 (m, 2H, arom. H), 6.91-6.96 (m, 2H, arom. H). ¹³C-NMR (125 MHz, CD₃COCD₃): 159.00, 158.80, 156.45, 148.50, 141.25, 139.80, 135.80, 135.70, 131.15, 130.65, 120.95, 118.95, 118.80, 116.95, 116.05, 114.50, 114.45. IR (neat) 3258, 1692, 1586, 1207, 781 cm⁻¹. MS (ESI): 263 (M−H)⁺.

### 128. 3,3′-Pyrazine-2,5-diyldiphenol (58)

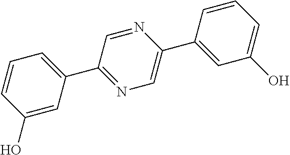

Synthesis: Suzuki cross coupling reaction (method A) followed by ether cleavage with boron tribromide (method E). Rf: (hexane/ethyl acetate 1:1): 0.31; ¹H NMR (CD₃COCD₃, 500 MHz): 8.93 (s, 1H, arom. H), 8.92 (s, 1H, arom. H), 8.79 (s, 1H, arom. H), 7.61 (m, 2H, arom. H), 7.38 (t, J=7.90 Hz, 1H, arom. H), 7.01 (dd, J=7.90 Hz and J=2.00 Hz, 1H, arom. H); ¹³C NMR (CD₃COCD₃, 125 MHz): 146.55, 142.00, 130.20, 117.95, 117.30, 113.60; IR (neat): 3321, 2959, 1607, 1456, 810 cm⁻¹; MS (ESI): 263 (M−H)⁺.

### 127. 3,3′-(1,2,4,5-Tetrazine-3,6-diyl)diphenol (59)

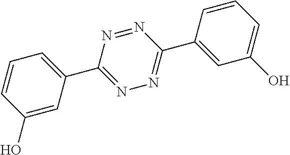

Synthesis: To a stirred mixture of 3-hydroxybenzonitrile (1.0 g, 8.4 mmol) and sulfur powder (135 mg, 4.2 mmol) in a few ml of ethanol, hydrazine monohydrate (0.8 ml, 16.8 mmol) is added, followed by heating under reflux for 2 h. After cooling to room temperature, sodium nitrite (926 mg) is added, followed by heating at 50° C. for another 2 h. The resulting suspension is filtered, and the solid is purified by column chromatography. Yield: 49 mg (6%), red solid. Rf (hexane/ethyl acetate 1:1): 0.57; ¹H-NMR (500 MHz, CD₃COCD₃): 7.36 (m, 2H, arom. H), 7.14-7.20 (m, 6H, arom. H); ¹³C-NMR (125 MHz, CD₃COCD₃): 158.70, 131.60, 124.10, 121.40, 119.40, 119.25, 113.85. IR (neat): 3362, 2239, 1583, 1283, 784, 678 cm⁻¹; MS (ESI): 265 (M−H)⁺.

### Example 2

Determination of the inhibitory activity of the potential inhibitors: Inhibition of 17β-HSD1 and 17β-HSD2: In both cases, human placenta served as the enzyme source (Lin, S.-X. et al., J. Biol. Chem., 267: 16182-16187 (1992)).

In the 17β-HSD1 test, NADH is employed as a cosubstrate at a final concentration of 500 μM in order to avoid the product inhibition occurring with NADPH. The enzyme preparation is diluted with test buffer to such an extent that the control conversion is 10 to maximally 20% (about 1:650). As the substrate, estrone in a final concentration of 500 nM is used, of which 3 nM is tritiated. 2,4,6,7-[³H]estrone is purchased from Perkin-Elmer, Boston. The inhibitor is added as a solution in DMSO (control: pure DMSO without inhibitor; the final concentration of DMSO in the assay is 1% in all cases). After the addition of the substrate, incubation is performed at 37° C. for 10 minutes, followed by quenching by the addition of HgCl₂ (final concentration of HgCl₂: 1.66 mM).

In the 17β-HSD2 test, the natural cosubstrate NAD⁺ is employed at a final concentration of 1500 μM. The microsome fraction is diluted in test buffer, so that a control conversion of 20 to 30% results (about 1:350). As the substrate, estradiol in a final concentration of 500 nM is used, of which 3 nM is tritiated. 2,4,6,7-[³H]estradiol is also purchased from Perkin-Elmer, Boston. The inhibitor is added as a solution in DMSO (control: pure DMSO without inhibitor; the final concentration of DMSO in the assay is 1 in all cases). After the addition of the substrate, incubation is performed at 37° C. for 20 minutes. The reaction is quenched by the addition of HgCl₂ (final concentration of HgCl₂: 0.166 mM).

After the reaction, the substrate and product are extracted by partitioning with ether, separated by chromatography (HPLC) and quantified by means of radiodetection. Compounds (1)-(7), (9)-(17), (21), (24)-(25), (27), (30), (35) and (39) do not show any inhibition of 17β-HSD1 at a concentration of 1 μM. Compound (24) shows 49% inhibition at 1 μM 17β-HSD1. The inhibitory activities of further compounds are expressed as IC₅₀ values and are summarized in Table 1.

TABLE 1
Inhibition of 17β-HSD1 and -HSD2

| | | | Selectivity |
|---|---|---|---|
| | 17β-HSD1 | 17β-HSD2 | IC₅₀ (17β-HSD2)/ |
| Compound | IC₅₀ (nM) | IC₅₀ (nM) | IC₅₀ (17β-HSD1) |
| 8 | 570 | 1020 | 1.8 |
| 18 | 560 | 800 | 1.4 |
| 19 | 50 | 4000 | 80 |
| 20 | 180 | 3100 | 17 |
| 22 | 280 | 2500 | 8.9 |
| 23 | 320 | n.d. | — |
| 26 | 410 | 2220 | 5.4 |
| 28 | 3400 | 1800 | 0.5 |
| 29 | 60 | 1950 | 33 |
| 31 | 110 | 750 | 6.8 |
| 32 | 130 | 1700 | 13 |
| 33 | 70 | 1270 | 18 |
| 34 | 170 | 560 | 3.3 |
| 36 | 510 | n.d. | — |
| 37 | 180 | 600 | 3.3 |
| 38 | 820 | n.d. | — |
| 40 | 360 | 2200 | 6.1 |
| 41 | 1600 | 2200 | 1.4 |
| 42 | 110 | 2300 | 21 |
| 43 | 1410 | 3100 | 2.2 |
| 44 | 240 | 4500 | 19 |
| 45 | 40 | 1970 | 49 |
| 46 | 800 | 1640 | 2.1 |
| 47 | 10 | 940 | 94 |
| 48 | 50 | 230 | 4.6 |
| 49 | 40 | n.d. | — |
| 50 | 530 | n.d. | — |
| 51 | 520 | n.d. | — |
| 52 | 340 | 2340 | 6.9 |
| 53 | 450 | n.d. | — |
| 54 | 720 | n.d. | — |
| 55 | 100 | 870 | 8.7 |
| 56 | 100 | 2020 | 20 |
| 57 | 100 | 3400 | 34 |
| 58 | 1000 | 5500 | 5.5 |
| 59 | 200 | 5100 | 26 |

n.d. = not determined

Affinity for the estrogen receptor α: The affinities of the inhibitors for estrogen receptor α were determined according to the method described by Zimmermann et al. (Zimmermann, J. et al., J. Steroid Biochem. Mol. Biol., 94: 57-66 (2005)). Slight changes were made: The respective inhibitor was incubated at RT for 2 h with shaking. After the addition of hydroxyapatite, the mixture was stored on ice for 15 min and vortexed every 5 min.

The receptor affinities are established as RBA (relative binding affinity) values. The RBA value of the reference estradiol is set to 100%. The inhibitors (19), (22), (31), (37), (47), (48), (49), (52), (55) and (57) were examined. In all cases, the RBA values are below 0.1%.

Drug interactions (inhibition of hepatic CYP enzymes): The inhibition of six human hepatic cytochrome P450 enzymes by selected compounds was examined by means of the kit supplied by Becton Dickinson GmbH (Heidelberg). The data are summarized in Table 2.

TABLE 2
Inhibition of hepatic CYP enzymes

| | IC50 (mean ± SD) [μM] | | | | | |
|---|---|---|---|---|---|---|
| Compound | CYP1A2 | CYP2B6 | CYP2C9 | CYP2C19 | CYP2D6 | CYP3A4 |
| 22 | 4.92 ± 0.09 | 14.22 ± 0.44 | 0.790 ± 0.018 | 4.57 ± 0.05 | 7.76 ± 0.03 | 1.95 ± 0.01 |
| 28 | 5.35 ± 0.24 | 18.82 ± 2.05 | 2.98 ± 0.02 | 3.37 ± 0.12 | 6.99 ± 0.02 | 7.36 ± 0.49 |
| 32 | 8.68 ± 0.29 | 11.21 ± 0.64 | 2.10 ± 0.09 | 4.43 ± 0.17 | 37.58 ± 1.00 | 0.84 ± 0.036 |
| 42 | 17.20 ± 1.16 | 7.68 ± 0.92 | 1.94 ± 0.10 | 4.20 ± 0.29 | 22.03 ± 0.29 | 2.13 ± 0.13 |
| Positive control | Furafyllin | Tranylcypromin | Sulfaphenazole | Tranylcypromine | Quinidine | Ketoconazole |
| IC₅₀ [μM] | 3.04 ± 0.08 | 6.96 ± 0.025 | 0.250 ± 0.027 | 3.04 ± 0.17 | 0.011 ± 0.001 | 0.054 ± 0.001 |

Performance of selected compounds in a CaCo2 assay: Caco-2 cell culture and transport experiments were performed according to Yee (Yee, S., Pharm. Res., 14(6): 763-766 (1997)), but slight modifications were introduced. The cultivation times were reduced from 21 to 10 days by increasing the sowing density from 6.3·10⁴ to 1.65·10⁵ cells per well. Four reference compounds (atenolol, testosterone, ketoprofene and erythromycin) were employed in each assay for evaluating the transport properties of the CaCo-2 cells. The initial concentration of the compounds in the donor compartment was 50 μM (in buffer with 1% ethanol or DMSO). Samples were taken from the acceptor side after 60, 120 and 180 min and from the donor side after 0 and 180 min. For glycoprotein P (P-gp) studies, bidirectional experiments were performed. The absorptive and secretory permeabilities (P_{app (a-b)} and P_{app (b-a)}) were determined. Thus, erythromycin was used as a substrate, and verapamil was used as an inhibitor of P-gp. Each experiment was performed in triplicate. The integrity of the monolayer was determined by means of TEER (transepithelial electric resistance) and the permeability for each assay was determined using Lucifer Yellow. All samples of the CaCo-2 transport experiments were analyzed by means of LC/MS/MS after dilution with buffer (1:1 with 2% acetic acid). The apparent permeability coefficient (Pₐₚₚ) was calculated by means of the formula given below, where dQ/dt represents the recovery rate of the mass in the acceptor compartment, A represents the surface area of the transwell membrane, and c₀ represents the initial concentration in the donor compartment. The data for selected inhibitors are summarized in Table 3.

$${P}_{app} = \frac{d Q}{d t \cdot A \cdot {c}_{0}}$$

TABLE 3

| | Pₐₚₚ | |
|---|---|---|
| Compound | [cm/sec ± rel. SD] | Permeability |
| 8 | (7.9 ± 0.8) · 10⁻⁶ | medium-high |
| 18 | (7.3 ± 0.9) · 10⁻⁶ | medium-high |
| 19 | (7.8 ± 2.5) · 10⁻⁶ | medium-high |
| 20 | (11.7 ± 8.1) · 10⁻⁶ | high |
| 28 | (11.5 ± 7.7) · 10⁻⁶ | high |
| 29 | (22.0 ± 1.0) · 10⁻⁶ | high |
| 32 | (14.4 ± 8.3) · 10⁻⁶ | high |
| 37 | (3.6 ± 0.5) · 10⁻⁶ | medium |
| 41 | (12.6 ± 7.2) · 10⁻⁶ | high |
| 42 | (12.5 ± 9.0) · 10⁻⁶ | high |
| 53 | (24.2 ± 6.9) · 10⁻⁶ | high |

Test for metabolic stability (rat liver microsomes): The stock solutions (10 mM in acetonitrile (AcCN)) are diluted to obtain working concentrations in 20% AcCN which are 10 times higher than the incubation concentrations of the compounds.

The incubation solution (180 μl) consists of 90 μl of a microsomal suspension of 0.33 mg/ml protein in 100 mM phosphate buffer, pH 7.4, with 90 μl NADP⁺-regenerating system (NADP⁺: 1 mM, glucose-6-phosphate 5 mM, glucose-6-phosphate dehydrogenase: 5 U/ml, MgCl₂ 5 mM).

The reaction is started by adding 20 μl of the compound to be tested in 20% AcCN to the microsome/buffer mixture preincubated at 37° C. After 0, 15, 30 and 60 minutes, 200 μl of sample solution is withdrawn and subjected to AcCN precipitation. The isolation of the compounds is effected by adding 200 μl of AcCN that contains the internal standard (1 μM) to 200 μl of sample solution and calibration standard. After shaking for 10 s and centrifugation at 4000 g, an aliquot of the supernatant is subjected to LC-MS/MS. Two controls are included: a positive control with 7-ethoxycoumarin as a reference to verify the microsomal enzyme activity, and a negative control in which microsomes are used that were heated for 25 minutes without a regenerating system, in order to ensure that the loss of substance is actually due to metabolization.

The amount of compound in a sample is expressed as the percent fraction of the compound remaining as compared to time t=0 (100%). The percent fraction is plotted versus time.

The thus established half lives of selected inhibitors and of the reference substances diazepam and diphenhydramine are summarized in Table 4.

TABLE 4

| Compound | Half life [min] |
|---|---|
| 22 | 12.6 |
| 28 | 10.6 |
| 32 | 18.6 |
| 42 | 22.7 |
| Diazepam | 40.77 |
| Diphenhydramine | 6.80 |

In vivo pharmacokinetics (rat): The compounds 29, 45, 47 and 59 and a reference compound were administered to adult male Wistar rats (n=4) perorally in a cassette dosing method (vehicle: Labrasol/water 1/1). The plasma profiles were established by means of LC-MS/MS. The data obtained are summarized in Table 5.

TABLE 5

| | Compound | | | | |
|---|---|---|---|---|---|
| | internal | | | | |
| Parameter | reference | 29 | 45 | 47 | 59 |
| Dose (mg/kg) | 10 | 10 | 10 | 10 | 10 |
| C_{max obs} (ng/kg) | 43.2 | 7.8 | 905.0 | 1388.2 | 106.0 |
| C_{z} (ng/kg) | 0.38 | 6.56 | 43.35 | 24.97 | 54.03 |
| t_{max obs} (h) | 2.0 | 8.0 | 4.0 | 8.0 | 3.0 |
| t_{z} (h) | 24.0 | 10.0 | 24.0 | 24.0 | 10.0 |
| t_{1/2z} (h) | 2.4 | 1.5 | 3.8 | 2.7 | 1.2 |
| AUC_{0-tz} (ng · h/ml) | 539.0 | 99.2 | 12037.3 | 19310.9 | 1204.5 |
| AUC_{0-∞} (ng · h/ml) | 540.3 | 99.2 | 12275.4 | 19407.1 | 1204.5 |

C_{max obs} highest measured concentration
C_{z} last analytically quantifiable concentration
t_{max obs} time to reach the highest measured concentration
t_{z} time to withdrawal of the last sample with analytically quantifiable concentration
t_{1/2z} half life (determined from the slope of the declining portion of the concentration vs. time curve
AUC_{0-tz} area below the concentration vs. time curve up to time t_{Z}
AUC_{0-∞} area below the concentration vs. time curve, extrapolated to ∞

Comparison of the inhibition data for isomeric bis(hydroxyphenyl)-1,3-thiazoles: When isomeric thiazoles are compared, only the para-/meta- or meta-/meta-substituted thiazoles show an inhibition of 17β-HSD1, while the para-/para-substituted compound 25, which is the sole of this series to be mentioned as an example in WO 00/19994, shows no activity (see Table 6). The affinities of the potent inhibitors 23, 24 and 26 for the estrogen receptor are negligible (data not stated).

TABLE 6

| | | Inhibition of | RBA (%) |
|---|---|---|---|
| Compound No. | | 17β-HSD1, | (Katzenellenbogen |
| HSD patent | Structure | IC₅₀ (nM) | patent) |
| 23 | 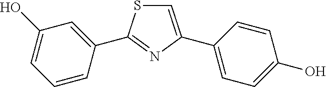 | 320 | not described |
| 24 | 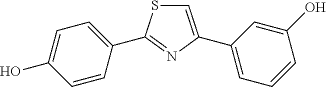 | 49% inhibition at 1 mM | not described |
| 25 | 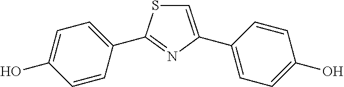 | no inhibition | 0.018 |
| 26 | 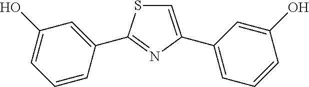 | 410 | not described |

1. A method of preventing or treating a hormone-related disease in a patient in need of such treatment, said method comprising administering to said patient an amount effective to treat said disease of a compound of formula (I): 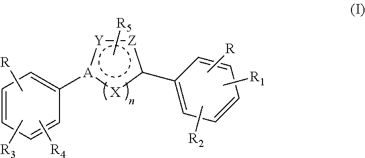
wherein
n is an integer selected from 0, 1 and 2;
A is C or N;
X is selected from CH, S, N, NH, —HC═N—, —N═CH— and O;
Y is selected from CH, —HC═CH—, S, N, O, NH and C═S;
Z is selected from CH, —HC═CH—, N, NH and O;
R are independently selected from halogen, hydroxy, —CN, —NO₂, —N(R′)₂, —SR′, alkyl, haloalkyl, alkoxy, haloalkoxy, aryl, heteroaryl, —SO₃R′, —NHSO₂R′, —R″—NHSO₂R′, —SO₂NHR′, —R″—SO₂NHR′, —NHCOR′, —CONHR′, —R″—NHCOR′, —R″—CONHR′, —COOR′, —OOCR′, —R″—COOR′, —R″—OOCR′, —CHNR′, —SO₂R′ and —SOR′, in which one of the radicals R is in a meta position and the other of the radicals R is in a meta or para position relative to the linkage with the central (hetero)aryl group;
R₁, R₂, R₃, R₄ and R₅ independently have the meaning as stated for R or are H;
R′ is selected from H, alkyl, aryl and heteroaryl;
R″ is selected from alkylene, arylene and heteroarylene;
wherein said alkyl, alkylene, aryl, arylene, heteroaryl and heteroarylene radicals in R, R₁, R₂, R₃, R₄, R₅, R′ and R″ may be substituted with 1 to 5 radicals R′″ and wherein the radicals R′″ are independently selected from halogen, hydroxy, —CN, alkyl, alkoxy, halogenated alkyl, halogenated alkoxy, —SH, alkylsulfanyl, arylsulfanyl, aryl, heteroaryl, —COOH, —COOalkyl, —CH₂OH, —NO₂ and —NH₂;
or a pharmacologically acceptable salt thereof.

2. The method according to claim 1, wherein
(i) n is 1, A is N, X is CH, Y is C═S and Z is NH; or
(ii) n is 1, A is N, X is CH, Y is CH and Z is N; or
(iii) n is 1, A is C, X is O or NH, Y is CH and Z is N; or
(iv) n is 1, A is C, X is N, Y is O and Z is CH; or
(v) n is 1, A is C, X is CH, Y is O and Z is N; or
(vi) n is 1, A is C, X is S, Y is N or CH and Z is CH; or
(vii) n is 1, A is C, X is N or CH, Y is S and Z is CH; or
(viii) n is 0, A is C, Y is S and Z is —HC═CH—; or
(ix) n is 1, A is C, X is CH, Y and Z are N and NH; or
(x) n is 1, A is C, X is S or O, Y and Z are N; or
(xi) n is 1, A is C, X and Z are N and Y is S; or
(xii) n is 2, A is C, X are CH, Y and Z are CH; or
(xiii) n is 1, A is C, X and Y are CH and Z is —HC═CH—; or
(xiv) n is 1, A is C, X is —N═CH—, Y is CH and Z is CH or N; or
(xv) n is 2, and X, Y and Z are N.

3. The method according to claim 1, wherein
(i) the radicals R are independently selected from halogen, hydroxy, —CN, —NO₂, —SH, —NHR′, —SO₃R′, alkyl, haloalkyl, alkoxy, haloalkoxy, alkylsulfanyl, aryl, heteroaryl, arylsulfanyl, —NHSO₂R′, —R″—NHSO₂R′, —SO₂NHR′, —R″—SO₂NHR′, —NHCOR′, —CONHR′, —R″—NHCOR′, —R″—CONHR′, —COOR′, —OOCR′, —R″—COOR′, —R″—OOCR′, —CHNR′, —SO₂R′ and —SOR′ (wherein R′ is H, lower alkyl or phenyl and R″ is lower alkylene or phenylene); and/or
(ii) the radicals R₁, R₂, R₃, R₄ and R₅ are independently selected from H, halogen, hydroxy, —CN, lower alkyl, halogenated lower alkyl, lower alkoxy, (lower alkyl)sulfanyl, aryl, heteroaryl, arylsulfanyl, —NHSO₂R′, —SO₂NHR′, —NHCOR′, —CONHR′, —COOR′, —OOCR′, —SO₂R′ and —SOR′ (wherein R′ is H, lower alkyl or phenyl).

4. The method according to claim 1, wherein
(i) the central aromatic ring in formula (I) is selected from a thiophene, thiazole, thiadiazole, benzene, pyridine and tetrazine ring; and/or
(ii) R are independently selected from halogen, hydroxy, —CN, —COOH, —NO₂, —NH₂, —SH, —SO₃H, SO₂NH₂, —NHSO₂-(lower alkyl), lower alkyl, halogenated lower alkyl, lower alkoxy and halogenated lower alkoxy; and/or
(iii) R₁, R₂, R₃, R₄ and R₅ are independently selected from H, halogen, halogenated lower alkyl and lower alkyl.

5. The method according to claim 1, wherein the compound of formula (I) is selected from,
4-(3-hydroxyphenyl)-1-(4-hydroxyphenyl)-1,3-dihydroimidazole-2-thione (1);
4-(4-hydroxyphenyl)-1-(3-hydroxyphenyl)-1,3-dihydroimidazole-2-thione (2);
3-[1-(4-hydroxyphenyl)-1H-imidazole-4-yl]phenol (4);
3-[4-(4-hydroxyphenyl)-1H-imidazole-4-yl]phenol (5);
3-[5-(4-hydroxyphenyl)-1,3-oxazole-2-yl]phenol (8);
3-[4-(4-hydroxyphenyl)-1,3-oxazole-2-yl]phenol (9);
3-[2-(4-hydroxyphenyl)-1H-imidazole-5-yl]phenol (10);
3-[5-(4-hydroxyphenyl)-1H-imidazole-2-yl]phenol (11);
3-[3-(4-hydroxyphenyl)-1H-pyrazole-5-yl]phenol (14);
3-[5-(4-hydroxyphenyl)-1H-pyrazole-3-yl]phenol (15);
3-[5-(4-hydroxyphenyl)isoxazole-3-yl]phenol (17);
3-[3-(4-hydroxyphenyl)isoxazole-5-yl]phenol (18);
3-[5-(4-hydroxyphenyl)-1,3-thiazole-2-yl]phenol (19);
3-[2-(4-hydroxyphenyl)-1,3-thiazole-5-yl]phenol (20);
3,3′-(1,3-thiazole-2,5-diyldiphenol (22);
3-[4-(4-hydroxyphenyl)-1,3-thiazole-2-yl]phenol (23);
3-[2-(4-hydroxyphenyl)-1,3-thiazole-4-yl]phenol (24);
3,3′-(1,3-thiazole-2,4-diyl)diphenol (26);
3-[3-(4-hydroxyphenyl)-2-thienyl]phenol (28);
3-[5-(4-hydroxyphenyl)-2-thienyl]phenol (29);
3,3′-thiene-2,5-diyldiphenol (31);
3-[5-(4-hydroxyphenyl)-3-thienyl]phenol (32);
3-[4-(4-hydroxyphenyl)-2-thienyl]phenol (33);
3,3′-thiene-2,4-diyldiphenol (34);
3,3′-(1,3,4-oxadiazole-2,5-diyl)diphenol (35);
3,3′-(1,3,4-thiadiazole-2,5-diyl)diphenol (36);
3,3′-(1,2,4-thiadiazole-2,5-diyl)diphenol (37);
3-[3-(4-methoxyphenyl)-1,2,4-thiadiazole-5-yl]phenol (38);
3-[3-(4-hydroxyphenyl)-1,2,4-thiadiazole-5-yl]phenol (40);
[1,1′,4′,1″]terphenyl-3,3′-diol (42);
[1,1′,3′,1″]terphenyl-4,3″-diol (43);
[1,1′,4′,1″]terphenyl-4,3″-diol (44);
4-[5-(3-hydroxyphenyl)-2-thienyl]-2-methylphenol (45);
4-[5-(3-hydroxyphenyl)-2-thienyl]benzene-1,2-diol (46);
2-fluoro-4-[5-(3-hydroxyphenyl)-2-thienyl]phenol (47);
2,6-difluoro-4-[5-(3-hydroxyphenyl)-2-thienyl]phenol (48);
4-[5-(3-hydroxyphenyl)-2-thienyl]-2-(trifluoromethyl)phenol (49);
3-[5-(3-fluorophenyl)-2-thienyl]phenol (50);
N-{3-[5-(3-hydroxyphenyl)-2-thienyl]phenyl}methanesulfonamide (51);
3-(5-phenyl-2-thienyl)phenol (52);
3-[5-(4-hydroxyphenyl)-2-thienyl]-5-methylphenol (53);
3-[5-(4-fluorophenyl)-2-thienyl]phenol (54);
4-[5-(3-hydroxyphenyl)-3-thienyl]-2-methylphenol (55);
4-[2-(3-hydroxyphenyl)-1,3-thiazol-5-yl]-2-methylphenol (56);
3,3′-pyridine-2,5-diyldiphenol (57); and
3,3′-(1,2,4,5-tetrazine-3,6-diyl)diphenol (59).

6. The method according to claim 1, wherein said hormone-related disease is selected from the group consisting of
(i) estrogen-related diseases; or
(ii) androgen-related diseases.

7. A compound of the formula (I) 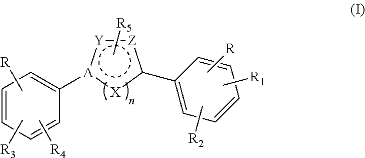
wherein
n is an integer selected from 0, 1 and 2;
A is C or N;
X is selected from CH, S, N, NH, —HC═N—, —N═CH— and O;
Y is selected from CH, —HC═CH—, S, N, O, NH and C═S;
Z is selected from CH, —HC═CH—, N, NH and O;
R are independently selected from halogen, hydroxy, —CN, —NO₂, —N(R′)₂, —SR′, alkyl, haloalkyl, alkoxy, haloalkoxy, aryl, heteroaryl, —SO₃R′, —NHSO₂R′, —R″—NHSO₂R′, —SO₂NHR′, —R″—SO₂NHR′, —NHCOR′, —CONHR′, —R″—NHCOR′, —R″—CONHR′, —COOR′, —OOCR′, —R″—COOR′, —R″—OOCR′, —CHNR′, —SO₂R′ and —SOR′, in which one of the radicals R is in a meta position and the other of the radicals R is in a meta or para position relative to the linkage with the central (hetero)aryl group;
R₁, R₂, R₃, R₄ and R₅ independently have the meaning as stated for R or are H;
R′ is selected from H, alkyl, aryl and heteroaryl;
R″ is selected from alkylene, arylene and heteroarylene;
wherein said alkyl, alkylene, aryl, arylene, heteroaryl and heteroarylene radicals in R, R₁, R₂, R₃, R₄, R₅, R′ and R″ may be substituted with 1 to 5 radicals R″ and wherein the radicals R′″ are independently selected from halogen, hydroxy, —CN, alkyl, alkoxy, halogenated alkyl, halogenated alkoxy, —SH, alkylsulfanyl, arylsulfanyl, aryl, heteroaryl, —COOH, —COOalkyl, —CH₂OH, —NO₂ and —NH₂;
with the proviso that if n is 1, A is C, X is —N═CH—, Y is CH, Z is N, R¹ to R⁴ are H and the radicals R are both OH or OOCCH₃, then the two radicals R are not both in meta positions; and
if n is 2, A is C, X, Y and Z are N, R¹ to R⁴ are H and the radicals R are both OH, then the two radicals R are not both in meta positions;
or a pharmacologically acceptable salt thereof.

8. The compound according to claim 7, wherein
(i) n is 1, A is N, X is CH, Y is C═S and Z is NH; or
(ii) n is 1, A is N, X is CH, Y is CH and Z is N; or
(iii) n is 1, A is C, X is O or NH, Y is CH and Z is N; or
(iv) n is 1, A is C, X is N, Y is O and Z is CH; or
(v) n is 1, A is C, X is CH, Y is O and Z is N; or
(vi) n is 1, A is C, X is S, Y is N or CH and Z is CH; or
(vii) n is 1, A is C, X is N or CH, Y is S and Z is CH; or
(viii) n is 0, A is C, Y is S and Z is —HC═CH—; or
(ix) n is 1, A is C, X is CH, Y and Z are N and NH; or
(x) n is 1, A is C, X is S or O, Y and Z are N; or
(xi) n is 1, A is C, X and Z are N and Y is S; or
(xii) n is 2, A is C, X are CH, Y and Z are CH; or
(xiii) n is 1, A is C, X and Y are CH and Z is —HC═CH—; or
(xiv) n is 1, A is C, X is —N═CH—, Y is CH and Z is CH or N; or
(xv) n is 2, and X, Y and Z are N.

9. The compound according to claim 7, which is selected from
4-(3-hydroxyphenyl)-1-(4-hydroxyphenyl)-1,3-dihydroimidazole-2-thione (1);
4-(4-hydroxyphenyl)-1-(3-hydroxyphenyl)-1,3-dihydroimidazole-2-thione (2);
3-[1-(4-hydroxyphenyl)-1H-imidazole-4-yl]phenol (4);
3-[4-(4-hydroxyphenyl)-1H-imidazole-4-yl]phenol (5);
3-[5-(4-hydroxyphenyl)-1,3-oxazole-2-yl]phenol (8);
3-[4-(4-hydroxyphenyl)-1,3-oxazole-2-yl]phenol (9);
3-[2-(4-hydroxyphenyl)-1H-imidazole-5-yl]phenol (10);
3-[5-(4-hydroxyphenyl)-1H-imidazole-2-yl]phenol (11);
3-[3-(4-hydroxyphenyl)-1H-pyrazole-5-yl]phenol (14);
3-[5-(4-hydroxyphenyl)-1H-pyrazole-3-yl]phenol (15);
3-[5-(4-hydroxyphenyl)isoxazole-3-yl]phenol (17);
3-[3-(4-hydroxyphenyl)isoxazole-5-yl]phenol (18);
3-[5-(4-hydroxyphenyl)-1,3-thiazole-2-yl]phenol (19);
3-[2-(4-hydroxyphenyl)-1,3-thiazole-5-yl]phenol (20);
3,3′-(1,3-thiazole-2,5-diyl)diphenol (22);
3-[4-(4-hydroxyphenyl)-1,3-thiazole-2-yl]phenol (23);
3-[2-(4-hydroxyphenyl)-1,3-thiazole-4-yl]phenol (24);
3,3′-(1,3-thiazole-2,4-diyl)diphenol (26);
3-[3-(4-hydroxyphenyl)-2-thienyl]phenol (28);
3-[5-(4-hydroxyphenyl)-2-thienyl]phenol (29);
3,3′-thiene-2,5-diyldiphenol (31);
3-[5-(4-hydroxyphenyl)-3-thienyl]phenol (32);
3-[4-(4-hydroxyphenyl)-2-thienyl]phenol (33);
3,3′-thiene-2,4-diyldiphenol (34);
3,3′-(1,3,4-oxadiazole-2,5-diyl)diphenol (35);
3,3′-(1,3,4-thiadiazole-2,5-diyl)diphenol (36);
3,3′-(1,2,4-thiadiazole-2,5-diyl)diphenol (37);
3-[3-(4-methoxyphenyl)-1,2,4-thiadiazole-5-yl]phenol (38);
3-[3-(4-hydroxyphenyl)-1,2,4-thiadiazole-5-yl]phenol (40);
[1,1′,4′,1″]terphenyl-3,3′-diol (42);
[1,1′,3′,1″]terphenyl-4,3″-diol (43);
[1,1′,4′,1″]terphenyl-4,3″-diol (44);
4-[5-(3-hydroxyphenyl)-2-thienyl]-2-methylphenol (45);
4-[5-(3-hydroxyphenyl)-2-thienyl]benzene-1,2-diol (46);
2-fluoro-4-[5-(3-hydroxyphenyl)-2-thienyl]phenol (47);
2,6-difluoro-4-[5-(3-hydroxyphenyl)-2-thienyl]phenol (48);
4-[5-(3-hydroxyphenyl)-2-thienyl]-2-(trifluoromethyl)phenol (49);
3-[5-(3-fluorophenyl)-2-thienyl]phenol (50);
N-{3-[5-(3-hydroxyphenyl)-2-thienyl]phenyl}methanesulfonamide (51);
3-(5-phenyl-2-thienyl)phenol (52);
3-[5-(4-hydroxyphenyl)-2-thienyl]-5-methylphenol (53);
3-[5-(4-fluorophenyl)-2-thienyl]phenol (54);
4-[5-(3-hydroxyphenyl)-3-thienyl]-2-methylphenol (55);
4-[2-(3-hydroxyphenyl)-1,3-thiazol-5-yl]-2-methylphenol (56); and
3,3′-pyridine-2,5-diyldiphenol (57).

10. A medicament or pharmaceutical composition comprising at least one compound according to claim 7 and optionally a pharmacologically suitable carrier.

11. The medicament or pharmaceutical composition according to claim 10, which is adapted for the treatment and prophylaxis of hormone-related, estrogen-related or androgen-related diseases.

12. The medicament or pharmaceutical composition according to claim 11, wherein said estrogen-related diseases are selected from endometriosis, endometrial carcinoma, adenomyosis and breast cancer.

13. The medicament or pharmaceutical composition according to claim 11, wherein said androgen-related diseases are selected from prostate carcinoma and benign prostate hyperplasia.

14. A process for preparing the compound according to claim 7, said comprising conducting a reaction according to the following reaction scheme: 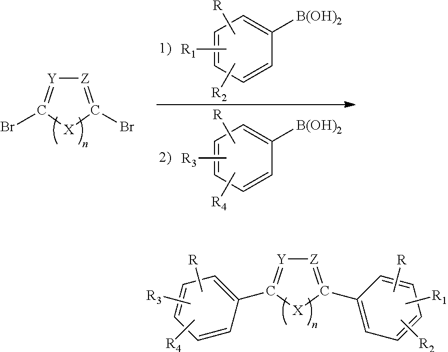
wherein the variables have the meanings as stated in claim 7.

15. (canceled)

16. The method according to claim 1, wherein said hormone-related disease is selected from the group consisting of estrogen-related diseases.
